(19) United States
(12) Patent Application Publication
(10) Pub. No.: US 2019/0016689 A1
(43) Pub. Date: Jan. 17, 2019

(54) NOVEL FLUORINATED QUINAZOLINE DERIVATIVES AS EGFR INHIBITORS

(71) Applicant: TRILLIUM THERAPEUTICS INC., Mississauga (CA)

(72) Inventors: Abdelmalik Slassi, Mississauga (CA); Peter Dove, Burlington (CA)

(73) Assignee: TRILLIUM THERAPEUTICS INC., Mississauga, ON (CA)

(21) Appl. No.: 16/066,574

(22) PCT Filed: Jan. 6, 2017
(86) PCT No.: PCT/CA2017/050015

§ 371 Date: Jun. 27, 2018

Related U.S. Application Data
(60) Provisional application No. 62/275,376, filed on Jan. 6, 2016.

(51) Int. Cl. C07D 239/94; C07D 401/12; C07D 413/12; A61P 35/00

(52) U.S. Cl. CPC C07D 239/94; A61P 35/00; C07D 413/12; C07D 401/12

(57) ABSTRACT

A novel class of fluorinated derivatives of Formula (I) have been prepared and found to be useful in the treatment of cancers and other EGFR related disorders.

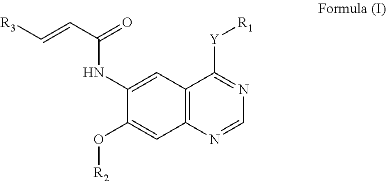

### CROSS-REFERENCE TO RELATED APPLICATIONS

The present application claims the benefit of priority from co-pending U.S. provisional patent application Ser. No. 62/275,376 filed on Jan. 6, 2016, the contents of which are incorporated herein by reference.

### FIELD

The present application relates to novel fluorinated quinazoline derivatives, to processes for their preparation, to compositions comprising them, and to their use in therapy. More particularly, it relates to compounds useful in the treatment of diseases, disorders or conditions mediated by epidermal growth factor receptor (EGFR). Such compounds and salts thereof may be useful in the treatment or prevention of a number of different cancers. The application also relates to pharmaceutical compositions comprising said compounds and salts thereof, especially useful polymorphic forms of these compounds and salts, intermediates useful in the manufacture of said compounds and to methods of treatment of diseases mediated by various different forms of EGFR using said compounds and salts thereof.

### BACKGROUND

Epidermal Growth Factor Receptor (EGFR) is a transmembrane protein tyrosine kinase member of the erbB receptor family. Upon binding of a growth factor ligand such as epidermal growth factor (EGF), the receptor can homo-dimerize with another EGFR molecule or hetero-dimerize with another family member such as erbB2 (HER2), erbB3 (HER3), or erbB4 (HER4). Homo- and/or hetero-dimerization of erbB receptors results in the phosphorylation of key tyrosine residues in the intracellular domain and leads to the stimulation of numerous intracellular signal transduction pathways involved in cell proliferation and survival. Deregulation of erbB family signalling promotes proliferation, invasion, metastasis, angiogenesis, and tumour cell survival and has been described in many human cancers, including those of the lung, head and neck and breast. The erbB family therefore represents a rational target for anticancer drug development and a number of agents targeting EGFR or erbB2 are now clinically available, including gefitinib (IRESSA™), erlotinib (TARCEVA™) and lapatinib (TYKERB™, TYVERB™). Detailed reviews of erbB receptor signalling and its involvement in tumourigenesis are provided in New England Journal of Medicine (2008) Vol. 358, 1160-74 and Biochemical and Biophysical Research Communications (2004) Vol. 319, I-II. In 2004 it was reported (Science Vol. 304, 1497-500 and New England Journal of Medicine [2004] Vol. 350, 2129-39) that activating mutations in EGFR correlated with response to gefitinib therapy in non-small-cell lung cancer (NSCLC).

The most common EGFR activating mutations, L858R and de1E746_A750, result in an increase in affinity for small molecule tyrosine kinase inhibitors such as gefitinib and erlotinib and a decrease in affinity for adenosine triphosphate (ATP) relative to wild type (WT) EGFR. Ultimately, acquired resistance to therapy with gefitinib or erlotinib arises, for example by mutation of the gatekeeper residue T790M, which is reportedly detected in 50% of clinically resistant patients. This mutation is not believed to hinder the binding of gefitinib or erlotinib to EGFR sterically, merely to alter the affinity to ATP to levels comparable to WT EGFR. In view of the importance of this mutation in resistance to existing therapies targeting EGFR, agents which inhibit EGFR harbouring the gatekeeper mutation may be especially useful in the treatment of cancer. There remains a need for compounds that exhibit favourable potency profiles against WT EGFR versus activating mutant forms of EGFR (for example the L858R EGFR mutant, or the de1E746_A750 mutant or the Exon19 deletion EGFR mutant) and/or resistant mutant forms of EGFR (for example T790M EGFR mutant), and/or selectivity over other enzyme receptors which may render the compounds especially promising for development as therapeutic agents. In this regard, there remains a need for compounds that show a higher inhibition of certain activating or resistance mutant forms of EGFR while at the same time showing relatively low inhibition of WT EGFR. Such compounds may be expected to be more suitable as therapeutic agents, particularly for the treatment of cancer, due to reduction of toxicity associated with WT EGFR inhibition. Such toxicity are known to manifest themselves in man as skin rashes and/or diarrhoea.

Glioblastoma multiforme (GBM) is the most aggressive of the astrocytic malignancies and the most common intracranial tumor in adults. Although the epidermal growth factor receptor (EGFR) is overexpressed and/or mutated in at least 50% of GBM cases and is required for tumor maintenance in animal models, EGFR inhibitors have thus far failed to deliver significant responses in GBM patients. One inherent resistance mechanism in GBM is the coactivation of multiple receptor tyrosine kinases, which generates redundancy in activation of phosphoinositide-3-kinase (PI3K) signaling. It has been demonstrated that the phosphatase and tensin homolog deleted on chromosome 10 (PTEN) tumor suppressor is frequently phosphorylated at a conserved tyrosine residue, Y240, in GBM clinical samples. Phosphorylation of Y240 is associated with shortened overall survival and resistance to EGFR inhibitor therapy in GBM patients and plays an active role in mediating resistance to EGFR inhibition in vitro. Y240 phosphorylation can be mediated by both fibroblast growth factor receptors and SRC family kinases (SFKs) but does not affect the ability of PTEN to antagonize PI3K signaling. These findings show that, in addition to genetic loss and mutation of PTEN, its modulation by tyrosine phosphorylation has important implications for the development and treatment of GBM.

P-glycoprotein (Pgp) is a member of the ABC-transporter family that transports substances across cellular membranes acting as an energy-dependent efflux pump extruding drugs out of the cells. Increased expression of Pgp in cancer cells is one of the major mechanisms of cancer resistances in chemotherapy and thus Pgp plays a key role on the pharmacokinetics of drug absorption and distribution. It is sometimes desirable for an anticancer drug to show low activity as a Pgp substrate.

The blood-brain barrier is a major impediment to the entry of many therapeutic drugs into the brain. Pgp is an ATP-dependent drug transport protein that is predominantly found in the apical membranes of a number of epithelial cell types in the body, including the blood luminal membrane of the brain capillary endothelial cells that make up the blood-brain barrier. Therefore drugs that are Pgp substrates will not be able to effectively cross the blood-brain barrier which will decrease their effectiveness as treatments for disorders of the CNS, such as brain cancer.

Fluorine has found interest in bioorganic and structural chemistry over the past decade and has become a useful feature in drug design. The small and highly electronegative fluorine atom can play a useful role in medicinal chemistry. Selective installation of fluorine into a therapeutic or diagnostic small molecule candidate can give a number of useful pharmacokinetic and/or physicochemical properties such as improved metabolic stability and enhanced membrane permeation. Increased binding affinity of fluorinated drug candidates to a target protein has also been documented in a number of cases. A further emerging application of the fluorine atom is the use of ¹⁸F as a radiolabel tracer atom in the sensitive technique of Positron Emission Tomography (PET) imaging.

Fluorine substitution has been investigated in drug research as a means of enhancing biological activity and/or increasing chemical and/or metabolic stability. Factors to be considered when synthesising fluorine-containing compounds include (a) the relatively small size of the fluorine atom (van der Waals radius of 1.47 Å), comparable to hydrogen (van der Waals radius of 1.20 Å), (b) the highly electron-withdrawing nature of fluorine, (c) the greater stability of the C—F bond compared to the C—H bond and (d) the greater lipophilicity of fluorine compared to hydrogen.

Despite the fact that fluorine is slightly larger than hydrogen, several studies have demonstrated that it is a reasonable hydrogen mimic and is expected to cause minimal steric perturbations with respect to the compound's mode of binding to a receptor or enzyme [Annu. Rev. Pharmacol. Toxicol. 2001, 41, 443-470]. However, the introduction of a fluorine atom can significantly alter the physicochemical properties of the compound due to its high electronegativity. Therefore this type of modification can induce altered and unpredictable biological responses of the molecule.

### SUMMARY

The Applicants have surprisingly found that one or more fluorine derived compounds have high potency against mutants of EGFR. Therefore, a novel class of fluorinated derivatives of Formula I have been prepared and found to be useful in the treatment of cancers and other EGFR related disorders.

Accordingly, the present application includes a compound of Formula I or a pharmaceutically acceptable salt, solvate and/or prodrug thereof:

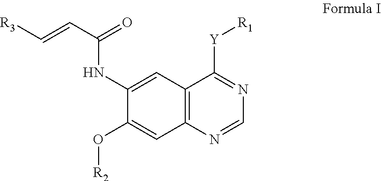

wherein:
R₁ is selected from aryl and heteroaryl both of which are unsubstituted or substituted with one or more substituents selected from halo, C₁₋₆alkyl, C₂₋₆alkenyl, C₂₋₆alkynyl, CN, CF₃, OR₆, SR₆, N(R₆)₂, and 3-7 membered heterocycloalkyl;
each R₆ is independently H, aryl, heteroaryl, C₁₋₆alkyl, C₂₋₆alkenyl or C₂₋₆alkynyl;
R₂ is selected from C₁₋₆alkyl,

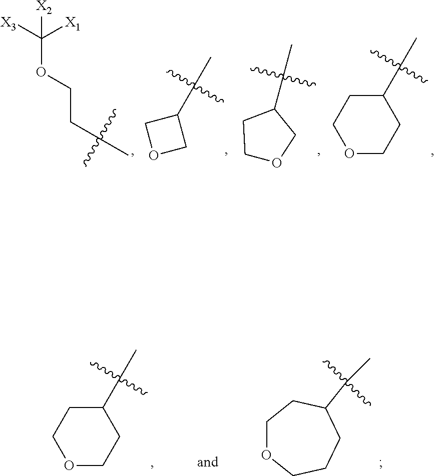

X₁, X₂ and X₃ are the same or different and are selected from H, halo and C₁₋₆alkyl R₃ is selected from H,

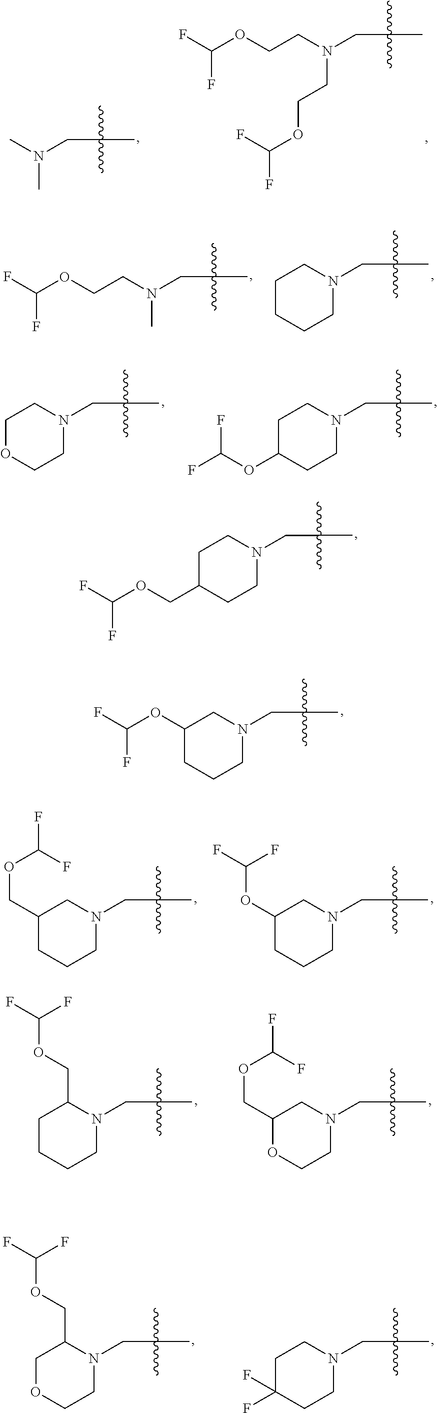

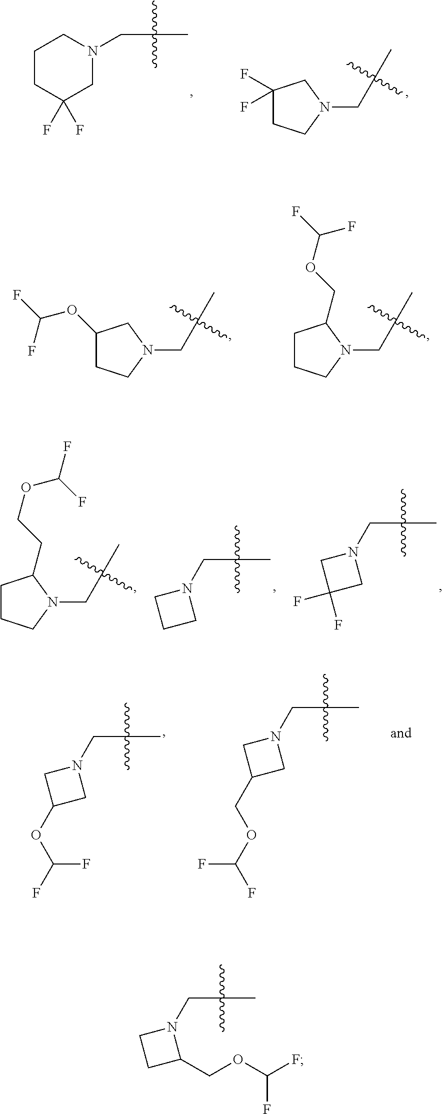

Y is selected from NH, O, S, SO and SO₂; and
at least one of R₂ and R₃ comprises a difluoromethyl group or at least R₃ comprises difluoromethylene group.

In some embodiments, the compound of Formula I or a pharmaceutically acceptable salt, solvate or prodrug thereof is:

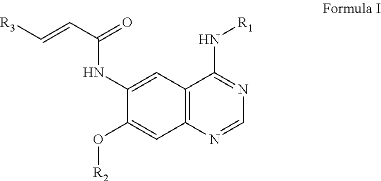

wherein:
R₁ represents aryl or heteroaryl (which may have one or more substituents selected from halo, CN, CF₃, OR₆, SR₆, N(R₆)₂, and 3-7 membered heterocycloalkyl; each R₆ is independently H or aryl, heteroaryl, C₁₋₆alkyl, alkenyl or alkynyl;
R₂ can be independently selected from

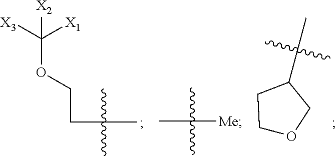

such that X₁, X₂, X₃ are the same or different and is selected from H, halo, lower alkyl;
R₃ can be independently selected from H,

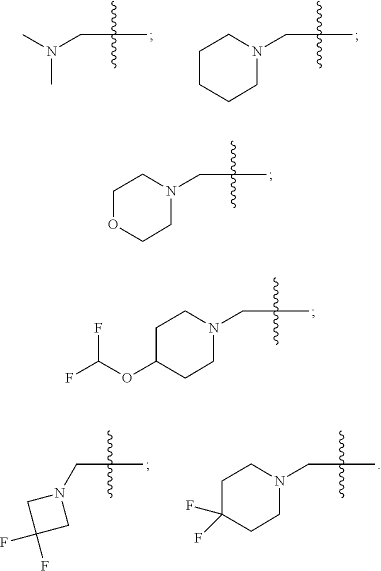

The present application also includes a composition comprising one or more compounds of the application and a carrier. In an embodiment, the composition is a pharmaceutical composition comprising one or more compounds of the application and a pharmaceutically acceptable carrier.

The compounds of the application have been shown to be capable of inhibiting EGFR protein function. Therefore the compounds of the application are useful for treating diseases, disorders or conditions treatable by inhibition of EGFR. Accordingly, the present application also includes a method of treating a disease, disorder or condition treatable by inhibition of EGFR, comprising administering a therapeutically effective amount of one or more compounds of the application to a subject in need thereof.

In a further embodiment, the compounds of the application are used as medicaments. Accordingly, the application also includes a compound of the application for use as a medicament.

The present application also includes a use of one or more compounds of the application for treatment of a disease, disorder or condition treatable by inhibition of EGFR as well as a use of one or more compounds of the application for the preparation of a medicament for treatment of a disease, disorder or condition treatable by inhibition of EGFR. The application further includes one or more compounds of the application for use in treating a disease, disorder or condition treatable by inhibition of EGFR.

The compounds of the application are useful for treating diseases, disorders or conditions mediated by EGFR inhibition. Accordingly, the present application also includes a method of treating a disease, disorder or condition mediated by EGFR protein inhibition, comprising administering a therapeutically effective amount of one or more compounds of the application to a subject in need thereof.

The present application also includes a use of one or more compounds of the application for treatment of a disease, disorder or condition mediated by EGFR protein inhibition as well as a use of one or more compounds of the application for the preparation of a medicament for treatment of a disease, disorder or condition mediated by EGFR protein inhibition. The application further includes one or more compounds of the application for use in treating a disease, disorder or condition mediated by EGFR protein inhibition.

In an embodiment, the disease, disorder or condition mediated by EGFR protein inhibition, or treatable by inhibition of EGFR, is a neoplastic disorder. In an embodiment, the treatment is in an amount effective to ameliorate at least one symptom of the neoplastic disorder, for example, reduced cell proliferation or reduced tumor mass in a subject in need of such treatment.

In an embodiment, the disease, disorder or condition mediated by EGFR protein inhibition, or treatable by inhibition of EGFR, is cancer.

In an embodiment, the disease, disorder or condition mediated by EGFR protein inhibition, or treatable by inhibition of EGFR, is a disease, disorder or condition associated with an uncontrolled and/or abnormal cellular activity affected directly or indirectly by EGFR. In another embodiment, the uncontrolled and/or abnormal cellular activity that is affected directly or indirectly by EGFR is proliferative activity in a cell.

The application also includes a method of inhibiting proliferative activity in a cell, comprising administering an effective amount of one or more compounds of the application to the cell.

In a further embodiment the disease, disorder or condition mediated by EGFR protein inhibition, or treatable by inhibition of EGFR, is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies.

The compound(s) of the application may also exhibit advantageous physical properties (for example higher permeability, enhanced CNS penetration and/or lower plasma protein binding) and/or favourable toxicity profiles (for example a decreased hERG blocking liability) and/or favourable metabolic profiles in comparison with other known EGFR/EGFR-mutant inhibitors. Therefore, such compound(s) may be especially useful in the treatment of disease states in which EGFR and/or activating mutations of EGFR and/or resistance mutations of EGFR are implicated, for example in the treatment of cancer.

Compounds of the application have been shown to be poor substrates for Pgp, resulting in their ability to effectively cross the blood brain barrier. In view of this, compounds of the application have shown significantly higher concentrations in the brain compared to the known compound, Afatinib, when administered both intravenously and orally.

Compounds of the application have also been shown to be selective inhibitors of the ephrin receptor EPHA6. Accordingly the present application also includes a method of treating a disease, disorder or condition treatable by inhibition of EPHA6, comprising administering a therapeutically effective amount of one or more compounds of the application to a subject in need thereof.

The present application also includes a use of one or more compounds of the application for treatment of a disease, disorder or condition treatable by inhibition of EPHA6 as well as a use of one or more compounds of the application for the preparation of a medicament for treatment of a disease, disorder or condition treatable by inhibition of EPHA6. The application further includes one or more compounds of the application for use in treating a disease, disorder or condition treatable by inhibition of EPHA6.

Among Ephs and ephrins, only EphA6 is consistently overexpressed in metastatic CaP (prostate cancer) cells. (Li, S. et al. Oncotarget, Vol. 6, No. 26, pp. 22587-22597). EphA6 has also been associated with colorectal cancer (Guda, K. et al. PNAS, 2015, 112(4):1149-1154). Accordingly in some embodiments, the disease, disorder or condition treatable by inhibition of EPHA6 is cancer, such as prostate cancer and colorectal cancer.

The application additionally provides a process for the preparation of compounds of Formula I. General and specific processes are discussed in more detail and set forth in the Examples below.

Other features and advantages of the present application will become apparent from the following detailed description. It should be understood, however, that the detailed description and the specific examples while indicating embodiments of the application are given by way of illustration only, since various changes and modifications within the spirit and scope of the application will become apparent to those skilled in the art from this detailed description.

### DETAILED DESCRIPTION

### I. Definitions

Unless otherwise indicated, the definitions and embodiments described in this and other sections are intended to be applicable to all embodiments and aspects of the application herein described for which they are suitable as would be understood by a person skilled in the art. Unless otherwise specified within this application or unless a person skilled in the art would understand otherwise, the nomenclature used in this application generally follows the examples and rules stated in “Nomenclature of Organic Chemistry” (Pergamon Press, 1979), Sections A, B, C, D, E, F, and H. Optionally, a name of a compound may be generated using a chemical naming program: ACD/ChemSketch, Version 5.09/September 2001, Advanced Chemistry Development, Inc., Toronto, Canada.

The term “compound of the application” or “compound of the present application” and the like as used herein refers to a compound of Formula I, and pharmaceutically acceptable salts, solvates and/or prodrugs thereof.

The term “composition of the application” or “composition of the present application” and the like as used herein refers to a composition comprising one or more compounds the application and at least one additional ingredient.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “suitable” as used herein means that the selection of the particular compound or conditions would depend on the specific synthetic manipulation to be performed, and the identity of the species to be transformed, but the selection would be well within the skill of a person trained in the art. All method steps described herein are to be conducted under conditions sufficient to provide the desired product. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form are included within the scope of the present application.

The compounds of the present application may further exist in varying polymorphic forms and it is contemplated that any polymorphs which form are included within the scope of the present application.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The expression “proceed to a sufficient extent” as used herein with reference to the reactions or method steps disclosed herein means that the reactions or method steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 100% of the starting material or substrate is converted to product.

The term “seven-membered” or “7-membered” as used herein as a prefix refers to a group having a ring that contains seven ring atoms.

The term “six-membered” or “6-membered” as used herein as a prefix refers to a group having a ring that contains six ring atoms.

The term “five-membered” or “5-membered” as used herein as a prefix refers to a group having a ring that contains five ring atoms.

The term “hydrocarbon” as used herein, whether it is used alone or as part of another group, refers to any structure comprising only carbon and hydrogen atoms up to 14 carbon atoms.

The term “hydrocarbon radical” or “hydrocarbyl” as used herein, whether it is used alone or as part of another group, refers to any structure derived as a result of removing a hydrogen atom from a hydrocarbon.

The term “hydrocarbylene” as used herein, whether it is used alone or as part of another group, refers to any structure derived as a result of removing a hydrogen atom from each end of a hydrocarbon.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₆alkyl means an alkyl group having 1, 2, 3, 4, 5 or 6 carbon atoms.

The term “alkylene” as used herein means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms.

The term “alkenyl” as used herein, whether it is used alone or as part of another group, means straight or branched chain, unsaturated alkenyl groups, i.e. alkyl groups containing at least one double bond. The number of carbon atoms that are possible in the referenced alkenyl groups are indicated by the prefix “Cₙ₁₋ₙ₂”. For example, the term C₂₋₆alkenyl means an alkenyl group having 2, 3, 4, 5 or 6 carbon atoms and at least one double bond.

The term “alkenylene” as used herein means straight or branched chain, unsaturated alkenylene group, that is an unsaturated carbon chain that contains substituents on two of its ends and at least one double bond. The number of carbon atoms that are possible in the referenced alkenylene groups are indicated by the prefix “Cₙ₁₋ₙ₂”. For example, the term C₂₋₆alkenylene means an alkenylene group having 2, 3, 4, 5 or 6 carbon atoms and at least 1, for example 1-3, 1-2 or 1 double bond.

The term “alkynyl” as used herein, whether it is used alone or as part of another group, means straight or branched chain unsaturated alkynyl groups, i.e. alkyl groups containing at least one triple bond. The number of carbon atoms that are possible in the referenced alkynyl group are indicated by the prefix “Cₙ₁₋ₙ₂”. For example, the term C₂₋₆alkynyl means an alkynyl group having 2, 3, 4, 5 or 6 carbon atoms and at least one triple bond.

The term “alkynylene” as used herein means straight or branched chain, unsaturated alkynylene group, that is an unsaturated carbon chain that contains substituents on two of its ends and at least one triple bond. The number of carbon atoms that are possible in the referenced alkylyne group are indicated by the prefix “Cₙ₁₋ₙ₂”. For example, the term C₂₋₆alkynylene means an alkynylene group having 2, 3, 4, 5 or 6 carbon atoms and at least 1, for example 1-3, 1-2 or 1 triple bond.

The term “haloalkyl” as used herein refers to an alkyl group wherein one or more, including all of the hydrogen atoms are replaced by a halogen atom. In an embodiment, the halogen is fluorine, in which case the haloalkyl is referred to herein as a “fluoroalkyl” group. In another embodiment, the haloalkyl comprises at least one —CHF₂ group.

The term “haloalkylene” as used herein refers to an alkylene group wherein one or more, including all of the hydrogen atoms are replaced by a halogen atom. In an embodiment, the halogen is fluorine, in which case the haloalkylene is referred to herein as a “fluoroalkylene” group. In another embodiment, the haloalkylene comprises a branched fluoroalkylene having at least one —CF₂— group.

The term “difluoromethyl group” as used herein refers to a chemical grouping of the formula

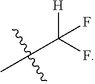 [SMILES: *C(F)F]

The term “difluoromethylene group” as used herein refers to a chemical grouping of the formula

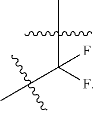 [SMILES: *C(*)(F)F]

The term “cyanoalkyl” as used herein refers to an alkyl group that is substituted by at least one cyano group. For example, the term C₁₋₁₀cyanoalkyl means an alkyl group having 1, 2, 3, 4, 5, 6, 7, 8, 9 or 10 carbon atoms and at least one cyano group attached thereto.

The term “alkoxy” as used herein, whether it is used alone or as part of another group, refers to the group “alkyl-O—”. The term C₁₋₁₀alkoxy means an alkyl group having 1, 2, 3, 4, 5, 6, 7, 8, 9 or 10 carbon atoms bonded to the oxygen atom of the alkoxy group. Exemplary alkoxy groups include without limitation methoxy, ethoxy, propoxy, isopropoxy, butoxy, t-butoxy and isobutoxy.

The term “alkenyloxy” as used herein, whether it is used alone or as part of another group, refers to the group “alkenyl-O—”. The term C₂₋₁₀alkenyloxy means an alkenyl group having 2, 3, 4, 5, 6, 7, 8, 9 or 10 carbon atoms and at least one double bond bonded to the oxygen atom of the alkenyloxy group. An exemplary alkoxy group is an allyloxy group.

The term “alkynyloxy” as used herein, whether it is used alone or as part of another group, refers to the group “alkynyl-O—”. The term C₂₋₁₀alkynyloxy means an alkynyl group having 2, 3, 4, 5, 6, 7, 8, 9 or 10 carbon atoms and at least one triple bond bonded to the oxygen atom of the alkynyloxy group. An exemplary alkoxy group is a propargyloxy group.

The term “aryloxy” as used herein, whether it is used alone or as part of another group, refers to the group “aryl-O—”. In an embodiment of the present disclosure, the aryl group contains 6, 9, 10 or 14 atoms such as phenyl, naphthyl, indanyl or anthracenyl.

The term “cycloalkyl,” as used herein, whether it is used alone or as part of another group, means saturated alkyl groups having at least one cyclic ring. The number of carbon atoms that are possible in the referenced cycloalkyl group are indicated by the numberical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “cycloalkylene” as used herein refers to a cycloalkyl group that contains substituents on two of its ends.

The term “aryl” as used herein, whether it is used alone or as part of another group, refers to carbocyclic groups that contain at least one aromatic ring. In an embodiment of the application, the aryl group contains from 6, 9, 10 or 14 atoms, such as phenyl, naphthyl, indanyl or anthracenyl.

The term “arylene” as used herein refers to an aryl group that contains substituents on two of its ends.

The term “heterocycloalkyl” as used herein, whether it is used alone or as part of another group, refers to a non-aromatic, ring-containing group having one or more multivalent heteroatoms, independently selected from the group consisting of N, O and S, as a part of the ring structure and including at least 3 and up to 20 atoms in the ring(s). Heterocycloalkyl groups are either saturated or unsaturated (i.e. contain one or more double bonds) and may contain more than one ring. When a heterocycloalkyl group contains more than one ring, the rings may be fused or unfused, or bridged or spirofused. When a heterocycloalkyl group contains the prefix “Cₙ₁₋ₙ₂” this prefix indicates the number of carbon atoms in the corresponding carbocyclic group, in which one or more, suitably 1 to 5, of the ring atoms is replaced with a heteroatom as defined above.

A first ring group being “fused” with a second ring group means the first ring and the second ring share at least two atoms therebetween.

Heterocycloalkyl includes monocyclic heterocycloalkyls such as but not limited to aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl. Additionally, heterocycloalkyl includes polycyclic heterocycloalkyls such as but not limited to pyrolizidinyl, and quinolizidinyl. In addition to the polycyclic heterocycloalkyls described above, heterocycloalkyl includes polycyclic heterocycloalkyls wherein the ring fusion between two or more rings includes more than one bond common to both rings and more than two atoms common to both rings. Examples of such bridged heterocycles include but are not limited to quinuclidinyl, diazabicyclo[2.2.1]heptyl and 7-oxabicyclo[2.2.1]heptyl.

The term “heteroaryl” as used herein means a monocyclic ring or a polycyclic ring system containing 5, 6, 7, 8, 9, 10, 11, 12, 13, 14, 15, 16, 17, 18, 19 or 20 atoms, of which one or more, for example 1 to 8, 1 to 6, 1 to 5, or 1 to 4, of the atoms are a heteromoiety selected from O, S, NH and NC₁₋₆alkyl, with the remaining atoms being C, CH or CH₂, said ring system containing at least one aromatic ring.

Heteroaryl includes for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4 oxadiazolyl.

Heteroaryl also includes polycyclic heteroaryls such as but not limited to indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl and acridinyl.

A five-membered heteroaryl is a heteroaryl with a ring having five ring atoms, where 1, 2 or 3 ring atoms are a heteromoiety selected from O, S, NH and NC₁₋₆alkyl, Exemplary five-membered heteroaryls include but are not limited to thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered heteroaryl is a heteroaryl with a ring having six ring atoms wherein 1, 2 or 3 ring atoms are a heteromoiety selected from O, S, NH and NC₁₋₆alkyl, Exemplary six-membered heteroaryls include but are not limited to pyridyl, pyrazinyl, pyrimidinyl, triazinyl and pyridazinyl.

The term “heteroarylene” as used herein refers to a heteroaryl group that contains substituents on two of its ends.

As a prefix, the term “substituted” as used herein refers to a structure, molecule or group in which one or more hydrogen atoms are replaced with one or more other chemical groups. In an embodiment, the chemical group is a C₁₋₄alkyl. In another embodiment, the chemical group is a C₁₋₁₂hydrocarbyl or a chemical group that contains one or more heteroatoms selected from N, O, S, F, Cl, Br, I, and P. Exemplary chemical groups containing one or more heteroatoms include heterocyclyl, —NO₂, —OR, —R′OR, —Cl, —Br, —I, —F, —CF₃, —C(═O)R, —NR₂, —SR, —SO₂R, —S(═O)R, —CN, —C(═O)OR, —C(═O)NR₂, —NRC(═O)R, —NRC(═O)OR, —R′NR₂, oxo (═O), imino (═NR), thio (═S), and oximino (═N—OR), wherein each “R” is hydrogen or a C₁₋₁₂hydrocarbyl and “R′” is a C₁₋₁₂hydrocarbylene. For example, substituted phenyl may refer to nitrophenyl, pyridylphenyl, methoxyphenyl, chlorophenyl, aminophenyl, etc., wherein the nitro, pyridyl, methoxy, chloro, and amino groups may replace any suitable hydrogen on the phenyl ring.

As a suffix, the term “substituted” as used herein in relation to a first structure, molecule or group, followed by one or more variables or names of chemical groups, refers to a second structure, molecule or group that results from replacing one or more hydrogens of the first structure, molecule or group with the one or more variables or named chemical groups. For example, a “phenyl substituted by nitro” refers to nitrophenyl.

The term “optionally substituted” refers to groups, structures, or molecules that are substituted or are not substituted (unsubstituted).

The term “amine” or “amino,” as used herein, whether it is used alone or as part of another group, refers to radicals of the general formula —NRR′, wherein R and R′ are each independently selected from hydrogen or a hydrocarbon radical.

The term “halo” as used herein refers to a halogen atom and includes fluoro, chloro, bromo and iodo.

The symbol “ [symbol] ” is used herein to represent the point of attachement of a group to the remainder of a molecule or chemical formula.

acac as used herein refers to acetylacetonate.

The term “atm” as used herein refers to atmosphere.

The term “aq.” as used herein refers to aqueous.

The terms “Boc” and “t-Boc” as used herein refer to the group tert-butoxycarbonyl.

DCM as used herein refers to dichloromethane.

DIPEA as used herein refers to N,N-Diisopropyl ethylamine

DMF as used herein refers to dimethylformamide.

DMSO as used herein refers to dimethylsulfoxide.

EDCI.HCl as used herein refers to N-[3-(dimethylamino)propyl]-N′-ethylcarbodiimide hydrochloride.

EDC as used herein refers to 1-ethyl-3-(3-dimethylaminopropyl)carbodiimide.

Et₂O as used herein refers to diethylether.

EtOAc as used herein refers to ethyl acetate.

Et as used herein refers to the group ethyl.

Fmoc as used herein refers to the group 9-fluorenylmethyloxycarbonyl.

The term “hr(s)” as used herein refers to hour(s).

The term “min(s)” as used herein refers to minute(s).

HOBt as used herein refers to N-hydroxybenzotriazole.

HBTU as used herein refers to O-(Benzotriazol-1-yl)-N,N,N′,N′-tetramethyluronium hexafluorophosphate.

MeOH as used herein refers to methanol.

Me as used herein refers to the group methyl.

t-BuLi as used herein refers to tert-butyllithium.

ON as used herein refers to overnight.

RT as used herein refers to room temperature.

TEA as used herein refers to triethylamine.

TFA as used herein refers to trifluoroacetic acid.

THF as used herein refers to tetrahydrofuran.

t-Bu as used herein refers to the group tertiary butyl.

SPE as used herein refers to solid phase extraction, for example using columns containing silica gel for mini-chromatography.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “cell” as used herein refers to a single cell or a plurality of cells and includes a cell either in a cell culture or in a subject.

The term “subject” as used herein includes all members of the animal kingdom including mammals, and suitably refers to humans. Thus the methods of the present application are applicable to both human therapy and veterinary applications. In an embodiment, the subject is a mammal. In another embodiment, the subject is human.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject. One non-limiting example of such a carrier is a pharmaceutically acceptable oil typically used for parenteral administration.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. Either the mono- or di-acid salts can be formed, and such salts can exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, EGFRaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. [See, for example, S. M. Berge, et al., “Pharmaceutical Salts,” J. Pharm. Sci. 1977, 66, 1-19]. The selection of the appropriate salt may be useful so that an ester functionality, if any, elsewhere in a compound is not hydrolyzed. The selection criteria for the appropriate salt will be known to one skilled in the art.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters.

The term “solvate” as used herein means a compound, or a salt or prodrug of a compound, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition described herein to prevent recurrence. Treatment methods comprise administering to a subject a therapeutically effective amount of one or more of the compounds of the application and optionally consist of a single administration, or alternatively comprise a series of administrations. For example, the compounds of the application may be administered at least once a week. However, in another embodiment, the compounds may be administered to the subject from about one time per three weeks, or about one time per week to about once daily for a given treatment. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration may be required. For example, the compounds are administered to the subject in an amount and for duration sufficient to treat the patient.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition mediated by EGFR protein inhibition or treatable by inhibition of EGFR, or manifesting a symptom associated with a disease, disorder or condition mediated by EGFR protein inhibition or treatable by inhibition of EGFR.

The “disease, disorder or condition mediated by EGFR” as used herein refers to a disease, disorder or condition treatable by inhibition of EGFR activity and particularly using an EGFR inhibitor, such as a compound of the application herein described.

The term “mediated by EGFR” as used herein means that the disease, disorder or condition to be treated is affected by, modulated by and/or has some biological basis, either direct or indirect, that includes aberrant EGFR activity, in particular, increased EGFR activity or, also, decreased EGFR activity such as results from mutation or splice variation and the like. These diseases respond favourably when EGFR activity associated with the disease is blocked by one or more of the present compounds.

As used herein, the term “effective amount” or “therapeutically effective amount” means an amount of a compound, or one or more compounds, of the application that is effective, at dosages and for periods of time necessary to achieve the desired result. For example in the context of treating a disease, disorder or condition mediated by EGFR protein inhibition or treatable by inhibition of EGFR, an effective amount is an amount that, for example, increases EGFR protein inhibition, or inhibits EGFR activity, compared to the inhibition without administration of the one or more compounds. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom. When the disease is cancer, amounts that are effective can cause a reduction in the number, growth rate, size and/or distribution of tumours.

The term “administered” as used herein means administration of a therapeutically effective amount of a compound, or one or more compounds, or a composition of the application to a cell either in cell culture or in a subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder. Neoplasms can be benign (such as uterine fibroids and melanocytic nevi), potentially malignant (such as carcinoma in situ) or malignant (i.e. cancer). Exemplary neoplastic disorders include but are not limited to carcinoma, sarcoma, metastatic disorders (e.g., tumors arising from the prostate), hematopoietic neoplastic disorders, (e.g., leukemias, lymphomas, myeloma and other malignant plasma cell disorders), metastatic tumors and other cancers. Prevalent cancers include breast, prostate, colon, lung, liver, brain, ovarian and pancreatic cancers.

The term “cancer” as used herein refers to cellular-proliferative disease states, including but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

The term “Afatinib” as used herein refers to the compound:

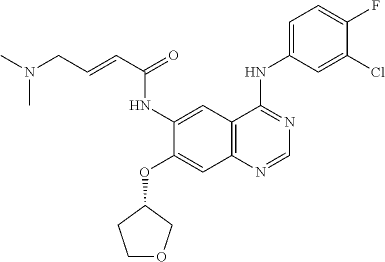 [SMILES: CN(C)C/C=C/C(=O)Nc1cc2c(Nc3ccc(F)c(Cl)c3)ncnc2cc1O[C@H]1CCOC1]

Afatinib (INN; trade name Gilotrif™ in the US and Giotrif™ in Europe) is a drug approved in United States, Europe, Taiwan, Mexico, Chile and Japan as well as other countries for the first-line treatment of patients with distinct types of metastatic (EGFR mutation positive) non-small cell lung carcinoma (NSCLC). This compound was developed by Boehringer Ingelheim. It acts as an irreversible covalent inhibitor of the receptor tyrosine kinases epidermal growth factor receptor (EGFR) and erbB-2 (HER2).

### II. Compounds and Compositions of the Application

Compounds of the present application were prepared and were found to inhibit uncontrolled and/or abnormal cellular activities affected directly or indirectly by EGFR protein. In particular, compounds of the present application exhibited activity as EGFR inhibitors, and are therefore useful in therapy, for example for the treatment of neoplastic disorders such as cancer. Compounds of the present application have been shown to be poor Pgp substrates and to have improved ability to cross the blood brain barrier, making them potential therapeutics for treating, for example, brain cancers.

The present application includes a compound of Formula I or a pharmaceutically acceptable salt, solvate and/or prodrug thereof:

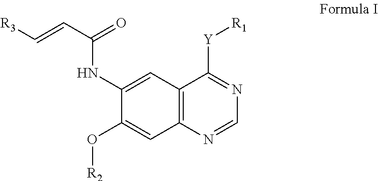

wherein:
R₁ is selected from aryl and heteroaryl both of which are unsubstituted or substituted with one or more substituents selected from halo, C₁₋₆alkyl, C₂₋₆alkenyl, C₂₋₆alkynyl, CN, CF₃, OR₆, SR₆, N(R₆)₂, and 3-7 membered heterocycloalkyl; each R₆ is independently H, aryl, heteroaryl, C₁₋₆alkyl, C₂₋₆alkenyl or C₂₋₆alkynyl; R₂ is selected from C₁₋₆alkyl,

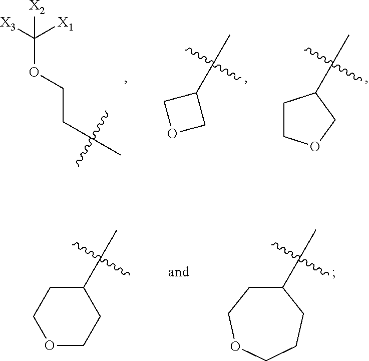

X₁, X₂ and X₃ are the same or different and are selected from H, halo and C₁₋₆alkyl R₃ is selected from H,

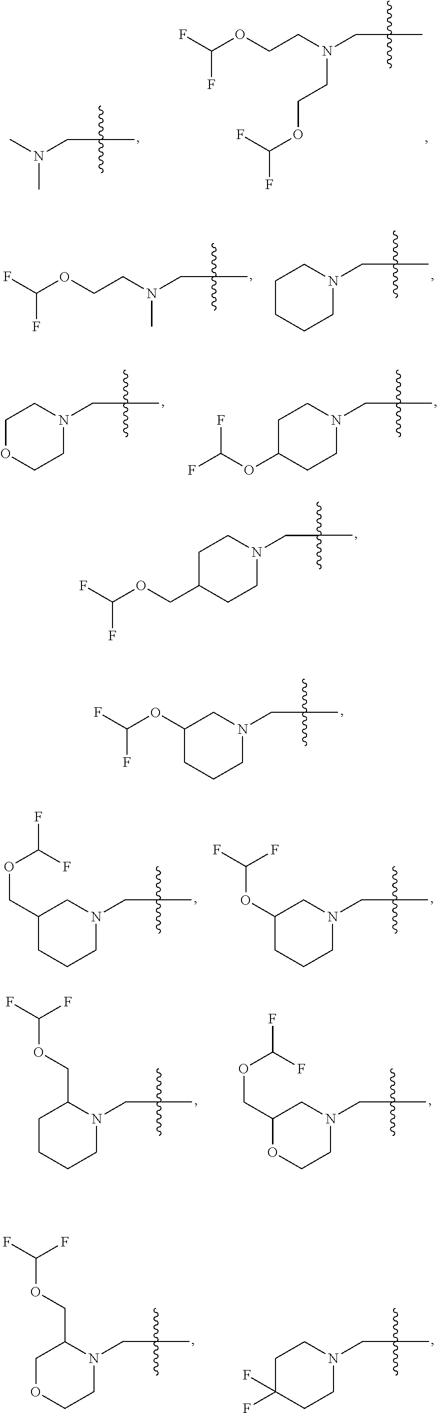

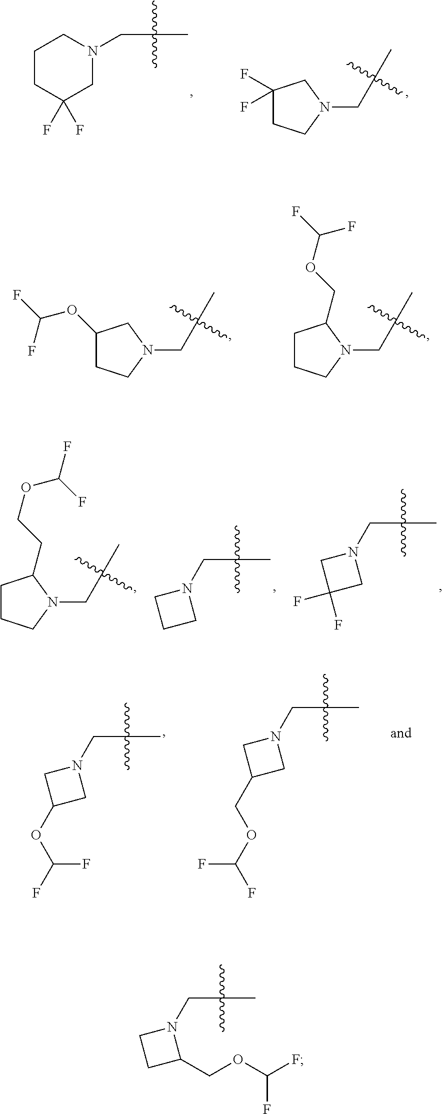

Y is selected from NH, O, S, SO and SO₂; and
at least one of R₂ and R₃ comprises a difluoromethyl group or at least R₃ comprises difluoromethylene group.

In some embodiments, R₁ is selected from unsubstituted or substituted aryl and unsubstituted or substituted heteroaryl, wherein the substituents for R₁ are selected from one to four of halo, C₁₋₆alkyl, haloC₁₋₆alkyl, CN, OR₆, N(R₆)₂, C₂₋₆alkynyl, and 5-6 membered heterocycloalkyl, in which R₆ is selected from haloC₁₋₆alkyl and C₁₋₆alkyl. In some embodiments, R₁ is selected from unsubstituted or substituted aryl wherein the substituents for R¹ are selected from one to four of one to four of halo, C₁₋₆alkyl, haloC₁₋₆alkyl, CN, OR₆, N(R₆)₂, C₂₋₆alkynyl, and 5-6 membered heterocycloalkyl, in which R₆ is selected from haloC₁₋₆alkyl and C₁₋₆alkyl. In some embodiments, R₁ is substituted aryl wherein the substituents of R₁ are selected from one to four of Cl, F, CF₃, OR₆, N(R₆)₂, C₂₋₆alkynyl, in which R₆ is selected from fluoroC₁₋₆alkyl and C₁₋₆alkyl. In some embodiments, R₁ is substituted phenyl wherein the substituents of R₁ are selected from one to three of Cl, F, CF₃, OR₆, N(R₆)₂, C₂₋₆alkynyl, in which R₆ is selected from CF₃, CHF₂ and CH₃. In some embodiments, R₁ is substituted phenyl wherein the substituents of R₁ are selected from one to three of Cl, F and C₂₋₆alkynyl.

In some embodiments, R₁ is selected from

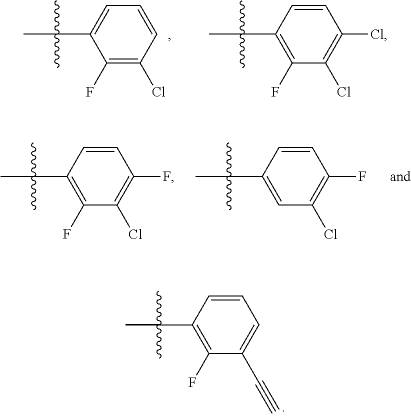

In some embodiments, R₁ is

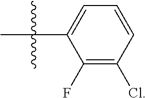 [SMILES: *c1cccc(Cl)c1F]

In some embodiments, R₁ is substituted heteroaryl wherein the substituents of R₁ are selected from one to three of Cl, F, CF₃, OR₆, N(R₆)₂, C₂₋₆alkynyl, in which R₆ is selected from fluoroC₁₋₆alkyl and C₁₋₆alkyl.

In some embodiments, R₁ represents aryl or heteroaryl, each which are unsubstituted or substituted with one or more substituents selected from halo, CN, CF₃, OR₆, SR₆, N(R₆)₂, and 3-7 membered heterocycloalkyl, wherein each R₆ is independently H or aryl, heteroaryl, C₁₋₆alkyl, alkenyl or alkynyl

In some embodiments, R₂ is selected from C₁₋₆alkyl and

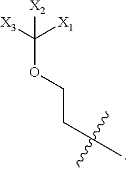

In some embodiments R₂ is

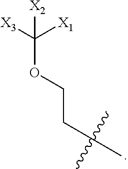

In some embodiments, R₂ is

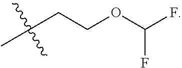 [SMILES: *CCOC(F)F]

In some embodiments, X₁, X₂ and X₃ are the same or different and are selected from H, F and C₁₋₄alkyl. In some embodiments, X₁, X₂ and X₃ are the same or different and are selected from H and F. In some embodiments, at least one of X₁, X₂ and X₃ is F.

In some embodiments, R₂ is selected from

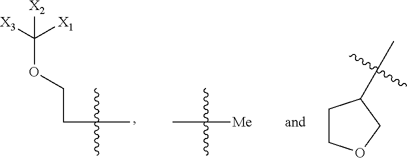

and X₁, X₂, X₃ are the same or different and are selected from H, halo, lower alkyl.

In some embodiments, R₃ is selected from:

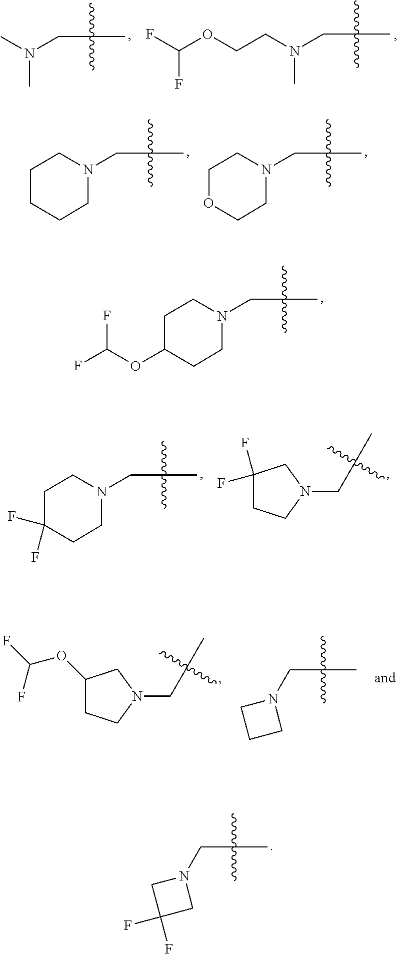

In some embodiment, R₃ is selected from:

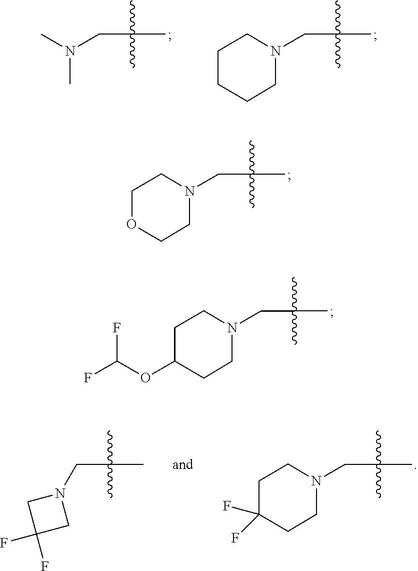

In some embodiments, R₃ is

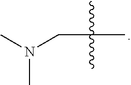 [SMILES: *CN(C)C]

In some embodiments, R₃ is

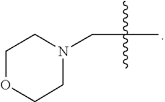 [SMILES: *CN1CCOCC1]

In some embodiments, at least one of R₂ and R₃ comprises a difluoromethyl group. In some embodiments, at least one of R₂ and R₃ comprises

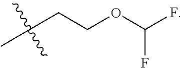 [SMILES: *CCOC(F)F]

In some embodiments, R₂ comprises a difluoromethyl group and R₃ comprises a difluoromethylene group.

In some embodiments, Y selected from O, NH and NCH₃. In some embodiments, Y is selected from O and NH. In some embodiments, Y is NH.

In an embodiment, the compound of the present application is selected from the compounds of Examples I-1 to I-30 as illustrated below or a pharmaceutically acceptable salt, solvate or prodrug thereof:

TABLE A

| Example | | |
|---|---|---|
| # | Structure | IUPAC Name |
| I-1 | 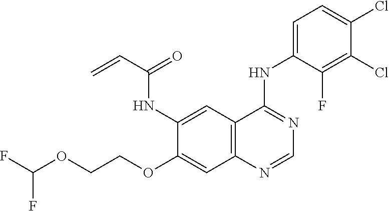 [SMILES: C=CC(=O)Nc1cc2c(Nc3ccc(Cl)c(Cl)c3F)ncnc2cc1OCCOC(F)F] | N-[4-(3,4-dichloro-2-fluoro- anilino)-7-[2- (difluoromethoxy)ethoxy]quinazolin- 6-yl]prop-2-enamides |
| I-2 | 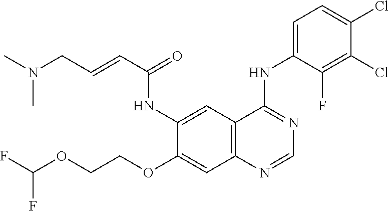 [SMILES: CN(C)C/C=C/C(=O)Nc1cc2c(Nc3ccc(Cl)c(Cl)c3F)ncnc2cc1OCCOC(F)F] | (E)-N-[4-(3,4-dichloro-2- fluoro-anilino)-7-[2- (difluoromethoxy)ethoxy]quinazolin- 6-yl]-4-(dimethylamino)but-2- enamide |
| I-3 | 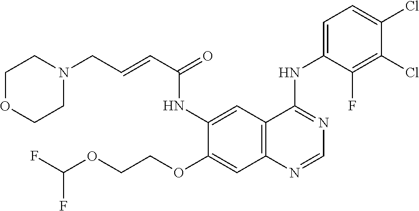 [SMILES: O=C(/C=C/CN1CCOCC1)Nc1cc2c(Nc3ccc(Cl)c(Cl)c3F)ncnc2cc1OCCOC(F)F] | (E)-N-[4-(3,4-dichloro-2- fluoro-anilino)-7-[2- (difluoromethoxy)ethoxy]quinazolin- 6-yl]-4-morpholino-but-2- enamide |
| I-4 | 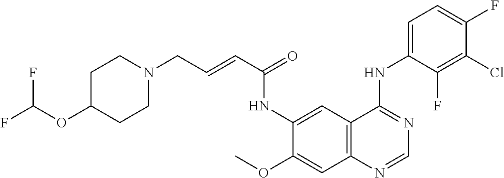 [SMILES: COc1cc2ncnc(Nc3ccc(F)c(Cl)c3F)c2cc1NC(=O)/C=C/CN1CCC(OC(F)F)CC1] | (E)-N-[4-(3-chloro-2,4-difluoro- anilino)-7-methoxy-quinazolin-6- yl]-4-[4-(difluoromethoxy)-1- piperidyl]but-2-enamide |
| I-5 | 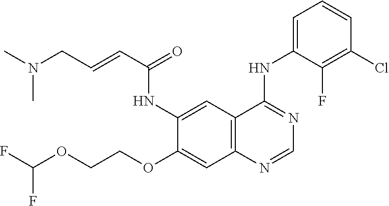 [SMILES: CN(C)C/C=C/C(=O)Nc1cc2c(Nc3cccc(Cl)c3F)ncnc2cc1OCCOC(F)F] | (E)-N-[4-(3-chloro-2-fluoro- anilino)-7-[2-(difluoromethoxy)- ethoxy]quinazolin-6-yl]-4- (dimethylamino)but-2-enamide |
| I-6 | 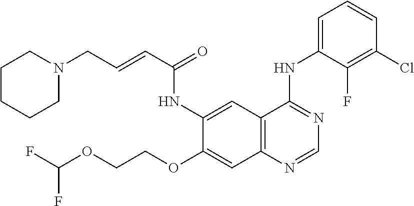 [SMILES: O=C(/C=C/CN1CCCCC1)Nc1cc2c(Nc3cccc(Cl)c3F)ncnc2cc1OCCOC(F)F] | (E)-N-[4-(3-chloro-2-fluoro- anilino)-7-[2-(difluoromethoxy)- ethoxy]quinazolin-6-yl]-4-(1- piperidyl)but-2-enamide |
| I-7 | 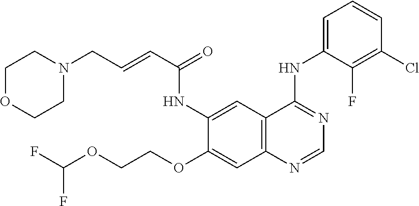 [SMILES: O=C(/C=C/CN1CCOCC1)Nc1cc2c(Nc3cccc(Cl)c3F)ncnc2cc1OCCOC(F)F] | (E)-N-[4-(3-chloro-2-fluoro- anilino)-7-[2-(difluoromethoxy)- ethoxy]quinazolin-6-yl]-4- morpholino-but-2-enamide |
| I-8 | 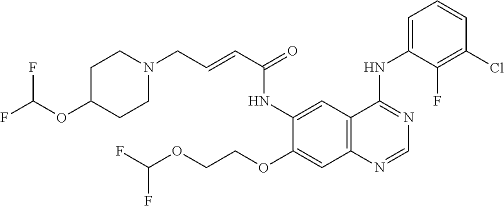 [SMILES: O=C(/C=C/CN1CCC(OC(F)F)CC1)Nc1cc2c(Nc3cccc(Cl)c3F)ncnc2cc1OCCOC(F)F] | (E)-N-[4-(3-chloro-2-fluoro- anilino)-7-[2-(difluoromethoxy)- ethoxy]quinazolin-6-yl]-4-[4- (difluoromethoxy)-1-piperidyl]but- 2-enamide |
| I-9 | 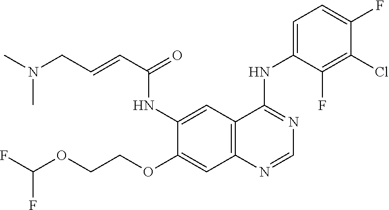 [SMILES: CN(C)C/C=C/C(=O)Nc1cc2c(Nc3ccc(F)c(Cl)c3F)ncnc2cc1OCCOC(F)F] | (E)-N-[4-(3-chloro-2,4-difluoro- anilino)-7-[2-(difluoromethoxy)- ethoxy]quinazolin-6-yl]-4- (dimethylamino)but-2-enamide |
| I-10 | 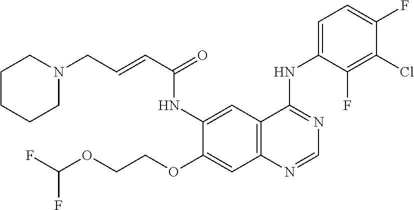 [SMILES: O=C(/C=C/CN1CCCCC1)Nc1cc2c(Nc3ccc(F)c(Cl)c3F)ncnc2cc1OCCOC(F)F] | (E)-N-[4-(3-chloro-2,4- difluoro-anilino)-7-[2- (difluoromethoxy)ethoxy]quinazolin- 6-yl]-4-(1-piperidyl)but-2- enamide |
| I-11 | 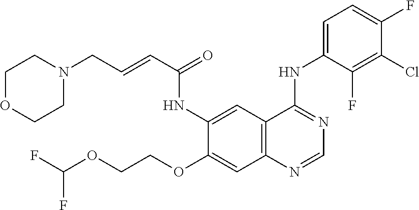 [SMILES: O=C(/C=C/CN1CCOCC1)Nc1cc2c(Nc3ccc(F)c(Cl)c3F)ncnc2cc1OCCOC(F)F] | (E)-N-[4-(3-chloro-2,4- difluoro-anilino)-7-[2- (difluoromethoxy)ethoxy]quinazolin- 6-yl]-4-morpholino-but-2- enamide |
| I-12 | 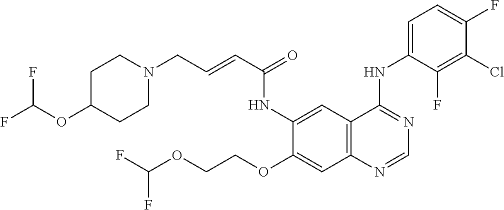 [SMILES: O=C(/C=C/CN1CCC(OC(F)F)CC1)Nc1cc2c(Nc3ccc(F)c(Cl)c3F)ncnc2cc1OCCOC(F)F] | (E)-N-[4-(3-chloro-2,4-difluoro- anilino)-7-[2-(difluoromethoxy)- ethoxy]quinazolin-6-yl]-4-[4- (difluoromethoxy)-1-piperidyl]but- 2-enamide |
| I-13 | 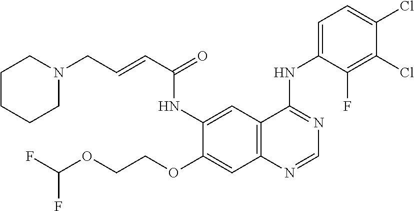 [SMILES: O=C(/C=C/CN1CCCCC1)Nc1cc2c(Nc3ccc(Cl)c(Cl)c3F)ncnc2cc1OCCOC(F)F] | (E)-N-[4-(3,4-dichloro-2- fluoro-anilino)-7-[2- (difluoromethoxy)ethoxy]quinazolin- 6-yl]-4-(1-piperidyl)but-2- enamide |
| I-14 | 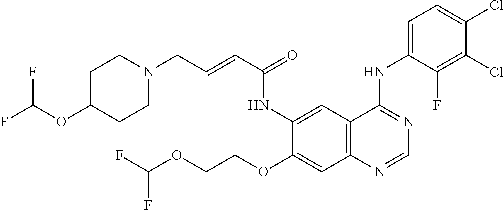 [SMILES: O=C(/C=C/CN1CCC(OC(F)F)CC1)Nc1cc2c(Nc3ccc(Cl)c(Cl)c3F)ncnc2cc1OCCOC(F)F] | (E)-N-[4-(3,4-dichlroo-2- fluoro-anilino)-7-[2- (difluoromethoxy)ethoxy]quinazolin- 6-yl]-4-[4-(difluoromethoxy)-1- piperidyl]but-2-enamide |
| I-15 | 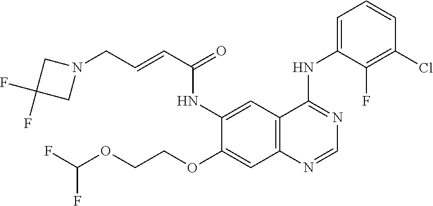 [SMILES: O=C(/C=C/CN1CC(F)(F)C1)Nc1cc2c(Nc3cccc(Cl)c3F)ncnc2cc1OCCOC(F)F] | (E)-N-[4-(3-chloro-2- fluoro-anilino)-7-[2- (difluoromethoxy)ethoxy]quinazolin- 6-yl]-4-(3,3-difluoroazetidin-1- yl)but-2-enamide |
| I-16 | 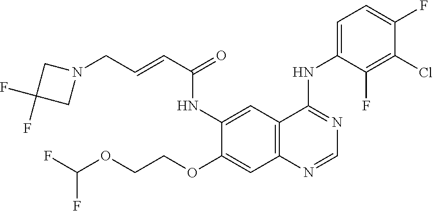 [SMILES: O=C(/C=C/CN1CC(F)(F)C1)Nc1cc2c(Nc3ccc(F)c(Cl)c3F)ncnc2cc1OCCOC(F)F] | (E)-N-[4-(3-chloro-2,4- difluoro-anilino)-7-[2- (difluoromethoxy)ethoxy]quinazolin- 6-yl]-4-(3,3-difluoroazetidin-1- yl)but-2-enamide |
| I-17 | 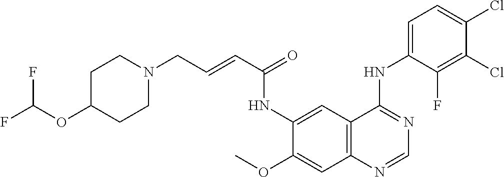 [SMILES: COc1cc2ncnc(Nc3ccc(Cl)c(Cl)c3F)c2cc1NC(=O)/C=C/CN1CCC(OC(F)F)CC1] | (E)-N-[4-(3,4-dichloro-2- fluoro-anilino)-7-methoxy- quinazolin-6-yl]-4-[4- (difluoromethoxy)-1-piperidyl]but- 2-enamide |
| I-18 | 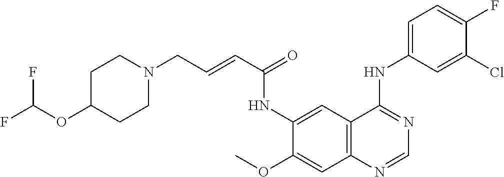 [SMILES: COc1cc2ncnc(Nc3ccc(F)c(Cl)c3)c2cc1NC(=O)/C=C/CN1CCC(OC(F)F)CC1] | (E)-N-[4-(3-chloro-4- fluoro-anilino)-7-methoxy- quinazolin-6-yl]-4-[4- (difluoromethoxy)-1-piperidyl]but- 2-enamide |
| I-19 | 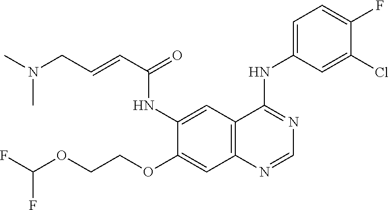 [SMILES: CN(C)C/C=C/C(=O)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy]quinazolin- 6-yl]-4-(dimethylamino)but-2- enamide |
| I-20 | 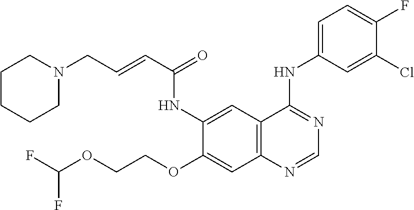 [SMILES: O=C(/C=C/CN1CCCCC1)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy]quinazolin- 6-yl]-4-(1-piperidyl)but-2- enamide |
| I-21 | 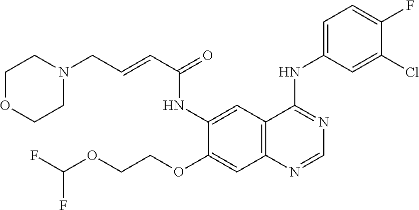 [SMILES: O=C(/C=C/CN1CCOCC1)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy]quinazolin- 6-yl]-4-morpholino-but-2- enamide |
| I-22 | 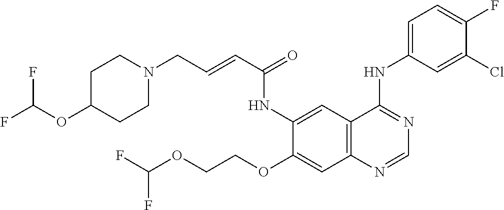 [SMILES: O=C(/C=C/CN1CCC(OC(F)F)CC1)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy]quinazolin- 6-yl]-4-[4-(difluoromethoxy)-1- piperidyl]but-2-enamide |
| I-23 | 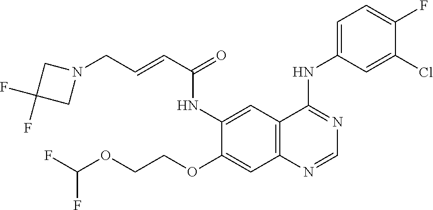 [SMILES: O=C(/C=C/CN1CC(F)(F)C1)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy]quinazolin- 6-yl]-4-(3,3-difluoroazetidin-1- yl)but-2-enamide |
| I-24 | 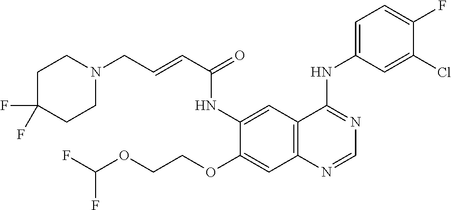 [SMILES: O=C(/C=C/CN1CCC(F)(F)CC1)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy]quinazolin- 6-yl]-4-(4,4-difluoro-1- piperidyl)but-2-enamide |
| I-25 | 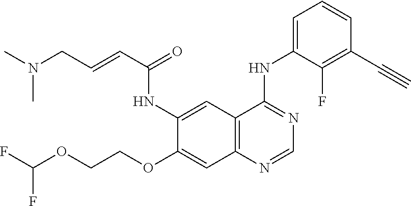 [SMILES: C#Cc1cccc(Nc2ncnc3cc(OCCOC(F)F)c(NC(=O)/C=C/CN(C)C)cc23)c1F] | (E)-N-[7-[2- (difluoromethoxy)- ethoxy]-4-(3-ethynyl-2-fluoro- anilino)quinazolin-6-yl]-4- (dimethylamino)but-2-enamide |
| I-26 | 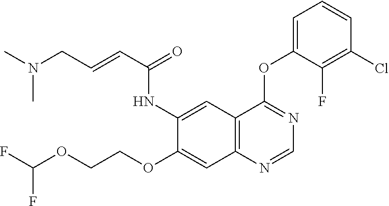 [SMILES: CN(C)C/C=C/C(=O)Nc1cc2c(Oc3cccc(Cl)c3F)ncnc2cc1OCCOC(F)F] | (E)-N-(4-(3-chloro-2- fluorophenoxy)-7-(2- (difluoromethoxy)ethoxy)quinazolin- 6-yl)-4-(dimethylamino)but-2- enamide |
| I-27 | 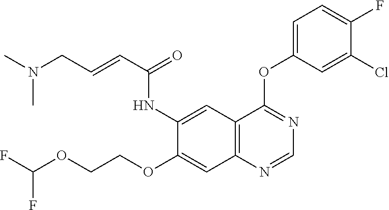 [SMILES: CN(C)C/C=C/C(=O)Nc1cc2c(Oc3ccc(F)c(Cl)c3)ncnc2cc1OCCOC(F)F] | (E)-N-(4-(3-chloro-4- fluorophenoxy)-7-(2- (difluoromethoxy)ethoxy)quinazolin- 6-yl)-4-(dimethylamino)but-2- enamide |
| I-28 | 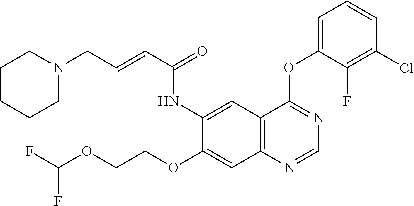 [SMILES: O=C(/C=C/CN1CCCCC1)Nc1cc2c(Oc3cccc(Cl)c3F)ncnc2cc1OCCOC(F)F] | (E)-N-(4-(3-chloro-2- fluorophenoxy)-7-(2- (difluoromethoxy)ethoxy)quinazolin- 6-yl)-4-(piperidin-1-yl)but-2- enamide |
| I-29 | 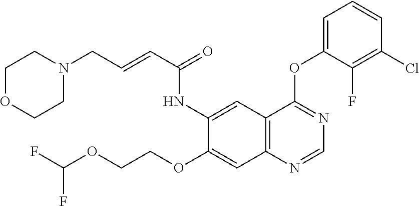 [SMILES: O=C(/C=C/CN1CCOCC1)Nc1cc2c(Oc3cccc(Cl)c3F)ncnc2cc1OCCOC(F)F] | (E)-N-(4-(3-chloro-2- fluorophenoxy)-7-(2- (difluoromethoxy)ethoxy)quinazolin- 6-yl)-4-morpholinobut-2- enamide |
| I-30 | 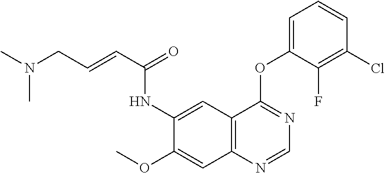 [SMILES: COc1cc2ncnc(Oc3cccc(Cl)c3F)c2cc1NC(=O)/C=C/CN(C)C] | (E)-N-(4-(3-chloro-2- fluorophenoxy)-7- methoxyquinazolin-6-yl)-4- (dimethylamino)but-2-enamide |

In some embodiments, the compound of Formula I is a compound of the formula:

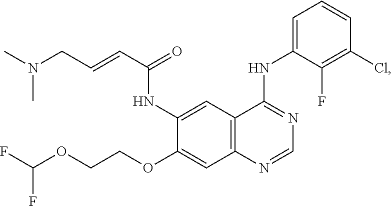 [SMILES: CN(C)C/C=C/C(=O)Nc1cc2c(Nc3cccc(Cl)c3F)ncnc2cc1OCCOC(F)F]

or a pharmaceutically acceptable salt and/or solvate thereof.

In some embodiments, the compound of Formula I is a compound of the formula:

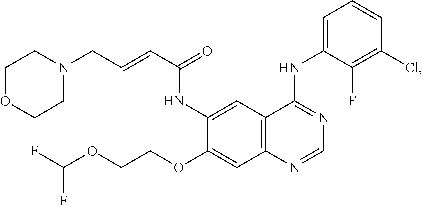 [SMILES: O=C(/C=C/CN1CCOCC1)Nc1cc2c(Nc3cccc(Cl)c3F)ncnc2cc1OCCOC(F)F]

or a pharmaceutically acceptable salt and/or solvate thereof.

In some embodiments, the compound of Formula I is a compound of the formula:

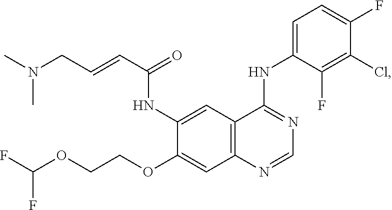 [SMILES: CN(C)C/C=C/C(=O)Nc1cc2c(Nc3ccc(F)c(Cl)c3F)ncnc2cc1OCCOC(F)F]

or a pharmaceutically acceptable salt and/or solvate thereof.

In some embodiments, the compound of Formula I is a compound of the formula:

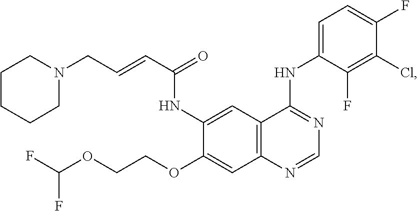 [SMILES: O=C(/C=C/CN1CCCCC1)Nc1cc2c(Nc3ccc(F)c(Cl)c3F)ncnc2cc1OCCOC(F)F]

or a pharmaceutically acceptable salt and/or solvate thereof.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, Pa., 1980, pp. 1530-1533 for further discussion of suppository dosage forms.

Compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compounds of the application including pharmaceutically acceptable salts, solvates and prodrugs thereof are suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of a pharmaceutically acceptable carrier, all percentages by weight being based on the total composition.

Compounds of the application may be used alone or in combination with other known agents useful for treating diseases, disorders or conditions mediated by EGFR protein inhibition, or that are treatable by inhibition of EGFR. When used in combination with other agents useful in treating diseases, disorders or conditions mediated by EGFR protein inhibition, or that are treatable by inhibition of EGFR, it is an embodiment that the compounds of the application are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, a compound of the present application is administered with another therapeutic agent simultaneously or sequentially in separate unit dosage forms or together in a single unit dosage form. Accordingly, the present application provides a single unit dosage form comprising one or more compounds of the application (e.g. a compound of Formula I), an additional therapeutic agent, and a pharmaceutically acceptable carrier.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily, weekly or monthly dose or the total daily dose may be divided into two, three or four daily doses.

To be clear, in the above, the term “a compound” also includes embodiments wherein one or more compounds are referenced.

### III. Preparation of Compounds of the Application

Compounds of the present application can be prepared by various synthetic processes. The choice of particular structural features and/or substituents may influence the selection of one process over another. The selection of a particular process to prepare a given compound of Formula I is within the purview of the person of skill in the art. Some starting materials for preparing compounds of the present application are available from commercial chemical sources. Other starting materials, for example as described below, are readily prepared from available precursors using straightforward transformations that are well known in the art.

The compounds of Formula I generally can be prepared according to the process illustrated in Schemes I and II. Variables in the following schemes are as defined above for Formula I unless otherwise specified.

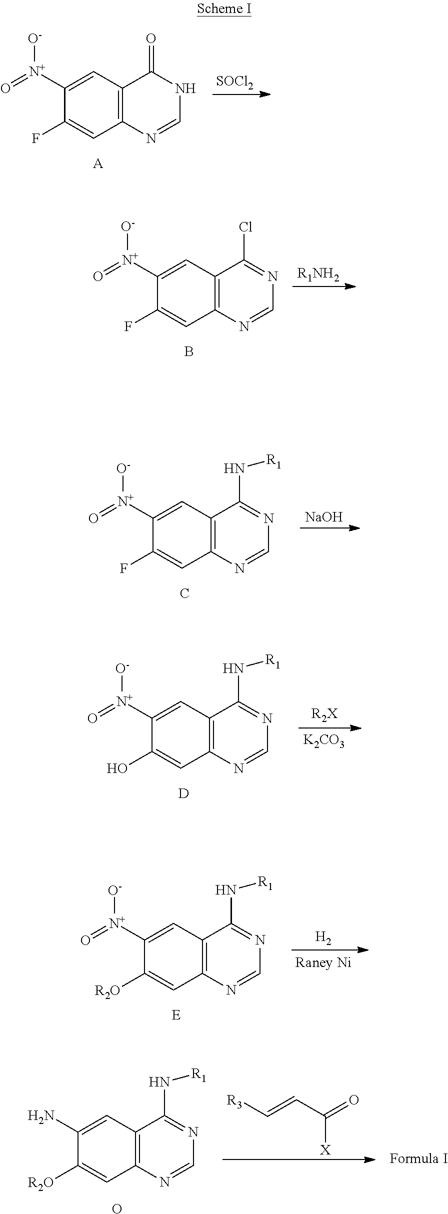

As shown in Scheme I, the compounds of the present application can be prepared by chlorination of 7-fluoro-6-nitroquinazolin-4-ol A with agents such as SOCl₂ or POCl₃ to give intermediate B. Subsequent treatment of chloride intermediate B with various amines or anilines give C. Nucleophilic aromatic substitution of C with a base such as Sodium hydroxide (NaOH) affords the Hydroxy-quinazoline derivative D. Alkylation of D with R₂X (X=leaving group such as halogens, Mesylates or Tosylates) under base-mediated conditions (eg, K₂CO₃ or Cs₂CO₃) affords intermediate E. Reduction of E under hydrogenation conditions with, for example, Raney nickel gives the amino-quinazoline F. Subsequent acylation with the acryloyl halide derivatives afford compounds of Formula I.

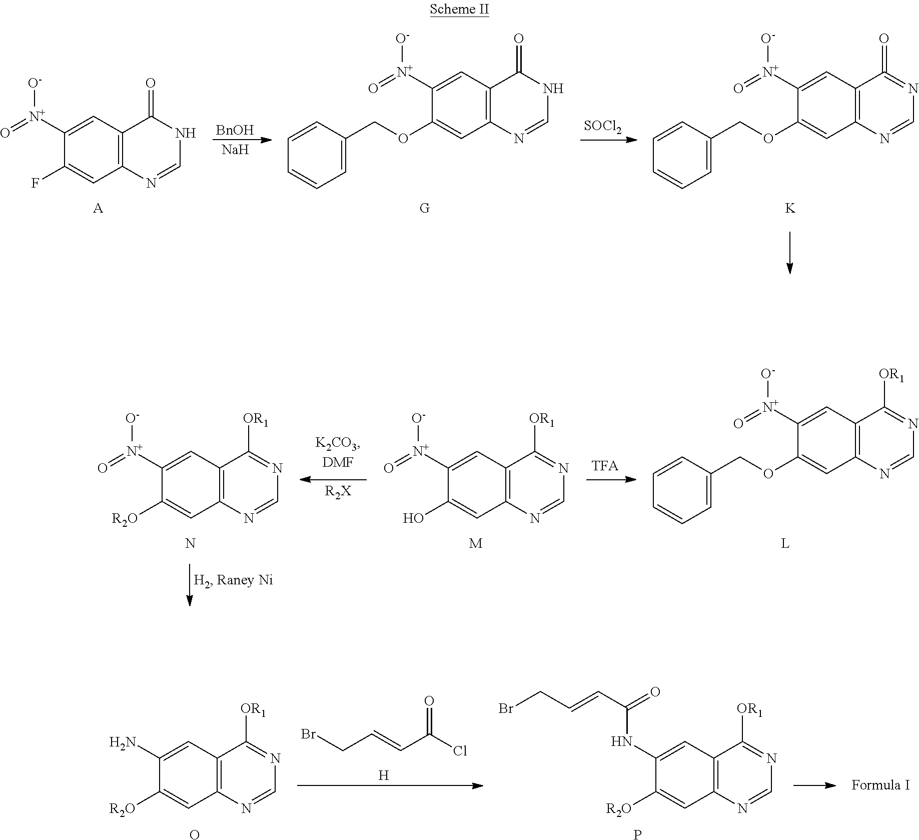

Alternatively, as shown is Scheme II, compounds of Formula I, when Y is oxygen can be prepared from A by displacing the fluorine atom with a benzyl alcohol under basic condition such as NaH to give the benzyloxy intermediate G then chlorinating with a suitable reagent such as SOCl₂ to give K. Subsequent displacement with various aromatic and heteroaromatic alcohols produces intermediate L which can then be deprotected under acidic conditions such as TFA to give the phenol intermediates M. The resulting product can be alkylated, as above in Scheme I, with R₂X (X=leaving group such as halogens, Mesylates or Tosylates) under base-mediated conditions (eg, K₂CO₃ or Cs₂CO₃) to afford intermediate N prior to undergoing a nitro reduction to obtain O. This material can be acylated with H to give the key intermediate bromide P which is easily substituted with amines to provide compounds of Formula I.

Alternatively, compounds of Formula I can also be prepared from intermediate F according to Scheme Ill. Bromination of the commercially available crotonic acid Q with NBS gives compound R which upon treatment with oxalyl chloride readily provides the acylating agents H. Treatment of intermediate F with the acylating agents H provides the penultimate precursor J. Subjecting J to various amines readily afford compounds of Formula I.

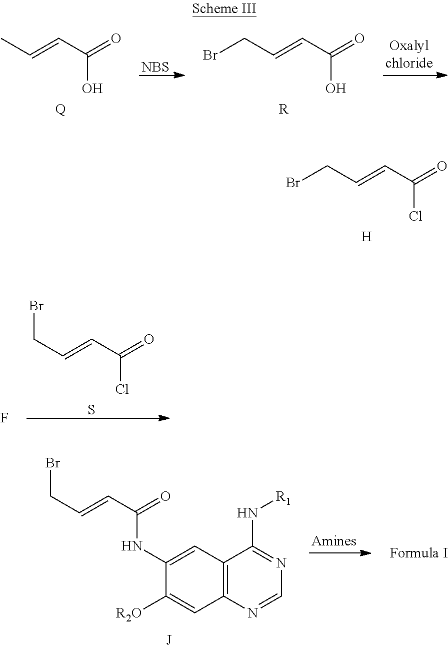

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### III. Methods and Uses of the Application

The compounds of the application have been shown to be capable of inhibiting EGFR activity, such as EGFR protein activity.

Accordingly, the present application includes a method for inhibiting EGFR in a cell, either in a biological sample or in a patient, comprising administering an effective amount of one or more compounds of the application to the cell. The application also includes a use of one or more compounds of the application for inhibiting EGFR in a cell as well as a use of one or more compounds of the application for the preparation of a medicament for inhibiting EGFR in a cell. The application further includes one or more compounds of the application for use in inhibiting EGFR in a cell.

As the compounds of the application have been shown to be capable of inhibiting EGFR protein activity, the compounds of the application are useful for treating diseases, disorders or conditions by inhibiting EGFR. Therefore the compounds of the present application are useful as medicaments. Accordingly, the present application includes a compound of the application for use as a medicament.

The present application also includes a method of treating a disease, disorder or condition by inhibition of EGFR comprising administering a therapeutically effective amount of one or more compounds of the application to a subject in need thereof.

The present application also includes a use of one or more compounds of the application for treatment of a disease, disorder or condition by inhibition of EGFR as well as a use of one or more compounds of the application for the preparation of a medicament for treatment of a disease, disorder or condition by inhibition of EGFR. The application further includes one or more compounds of the application for use in treating a disease, disorder or condition by inhibition of EGFR.

In an embodiment, the disease, disorder or condition is a neoplastic disorder. Accordingly, the present application also includes a method of treating a neoplastic disorder comprising administering a therapeutically effective amount of one or more compounds of the application to a subject in need thereof. The present application also includes a use of one or more compounds of the application for treatment of a neoplastic disorder as well as a use of one or more compounds of the application for the preparation of a medicament for treatment of a neoplastic disorder. The application further includes one or more compounds of the application for use in treating a neoplastic disorder. In an embodiment, the treatment is in an amount effective to ameliorate at least one symptom of the neoplastic disorder, for example, reduced cell proliferation or reduced tumor mass, among others, in a subject in need of such treatment.

Compounds of the application have been demonstrated to be effective against the cell lines of a 60 human tumor cell line panel. Therefore in another embodiment of the present application, the disease, disorder or condition that is treated by inhibition EGFR is cancer. Accordingly, the present application also includes a method of treating cancer comprising administering a therapeutically effective amount of one or more compounds of the application to a subject in need thereof. The present application also includes a use of one or more compounds of the application for treatment of cancer as well as a use of one or more compounds of the application for the preparation of a medicament for treatment of cancer. The application further includes one or more compounds of the application for use in treating cancer. In an embodiment, the compound is administered for the prevention of cancer in a subject such as a mammal having a predisposition for cancer.

In an embodiment, the cancer is selected from a cancer of the skin, blood, prostate, colorectum, pancreas, kidney, ovary, breast, for example mammary, liver, tongue and lung. In another embodiment, the cancer is selected from leukaemia, lymphoma, non-Hodgkin's lymphoma and multiple myeloma. In a further embodiment of the present application, the cancer is selected from leukemia, melanoma, lung cancer, colon cancer, brain cancer, ovarian cancer, breast cancer, prostate cancer and kidney cancer. In a further embodiment, the cancer is brain cancer. In some embodiments, the brain cancer is glioblastoma multiforme.

In an embodiment, the disease, disorder or condition that is treated by inhibition of EGFR is a disease, disorder or condition associated with an uncontrolled and/or abnormal cellular activity affected directly or indirectly by inhibition of EGFR. In another embodiment, the uncontrolled and/or abnormal cellular activity that is affected directly or indirectly by inhibition of EGFR is proliferative activity in a cell. Accordingly, the application also includes a method of inhibiting proliferative activity in a cell, comprising administering an effective amount of one or more compounds of the application to the cell. The present application also includes a use of one or more compounds of the application for inhibition of proliferative activity in a cell as well as a use of one or more compounds of the application for the preparation of a medicament for inhibition of proliferative activity in a cell. The application further includes one or more compounds of the application for use in inhibiting proliferative activity in a cell.

The present application also includes a method of inhibiting uncontrolled and/or abnormal cellular activities affected directly or indirectly by EGFR protein in a cell, either in a biological sample or in a subject, comprising administering an effective amount of one or more compounds of the application to the cell. The application also includes a use of one or more compounds of the application for inhibition of uncontrolled and/or abnormal cellular activities affected directly or indirectly by EGFR protein in a cell as well as a use of one or more compounds of the application for the preparation of a medicament for inhibition of uncontrolled and/or abnormal cellular activities affected directly or indirectly by EGFR protein in a cell. The application further includes one or more compounds of the application for use in inhibiting uncontrolled and/or abnormal cellular activities affected directly or indirectly by EGFR protein in a cell.

Accordingly, the present application also includes a method of treating a disease, disorder or condition that is treatable by inhibition of EGFR comprising administering a therapeutically effective amount of one or more compounds of the application in combination with another known agent useful for treatment of a disease, disorder or condition treatable by inhibition of EGFR to a subject in need thereof. The present application also includes a use of one or more compounds of the application in combination with another known agent useful for treatment of a disease, disorder or condition treatable by inhibition of EGFR for treatment of a disease, disorder or condition treatable by inhibition of EGFR, as well as a use of one or more compounds of the application in combination with another known agent useful for treatment of a disease, disorder or condition treatable by inhibition of EGFR for the preparation of a medicament for treatment of a disease, disorder or condition treatable by inhibition of EGFR. The application further includes one or more compounds of the application in combination with another known agent useful for treatment of a disease, disorder or condition treatable by inhibition of EGFR for use in treating a disease, disorder or condition treatable by inhibition of EGFR. In an embodiment, the disease, disorder or condition treatable by inhibition of EGFR is cancer such as multiple myeloma, lymphoma, leukemia, ovarian cancer, brain cancer, lung cancer, pancreatic cancer and brain cancers.

In a further embodiment, the disease, disorder or condition treatable by inhibition of EGFR is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies.

The following non-limiting examples are illustrative of the present application:

### EXAMPLES

### General Methods

All starting materials used herein were commercially available or earlier described in the literature. The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as an internal reference, in deuterated chloroform as solvent unless otherwise indicated. All reported chemical shifts are in ppm on the delta-scale, and the fine splitting of the signals as appearing in the recordings is generally indicated, for example as s: singlet, br s: broad singlet, d: doublet, t: triplet, q: quartet, m: multiplet. Unless otherwise indicated, in the tables below, ¹H NMR data was obtained at 400 MHz, using CDCl₃ as the solvent.

Purification of products was carried out using Chem Elut Extraction Columns (Varian, cat #1219-8002), Mega BE-SI (Bond Elut Silica) SPE Columns (Varian, cat #12256018; 12256026; 12256034) or by flash chromatography in silica-filled glass columns.

### Preparation of Intermediates and Examples

Scheme IV outlines the synthesis of compounds of Formula I, represented by I-7, when R₁ is 3-chloro-2-fluoroaniline group, R₂ is methyl-difluoromethoxy moiety and R₃ is methylene-morpholine.

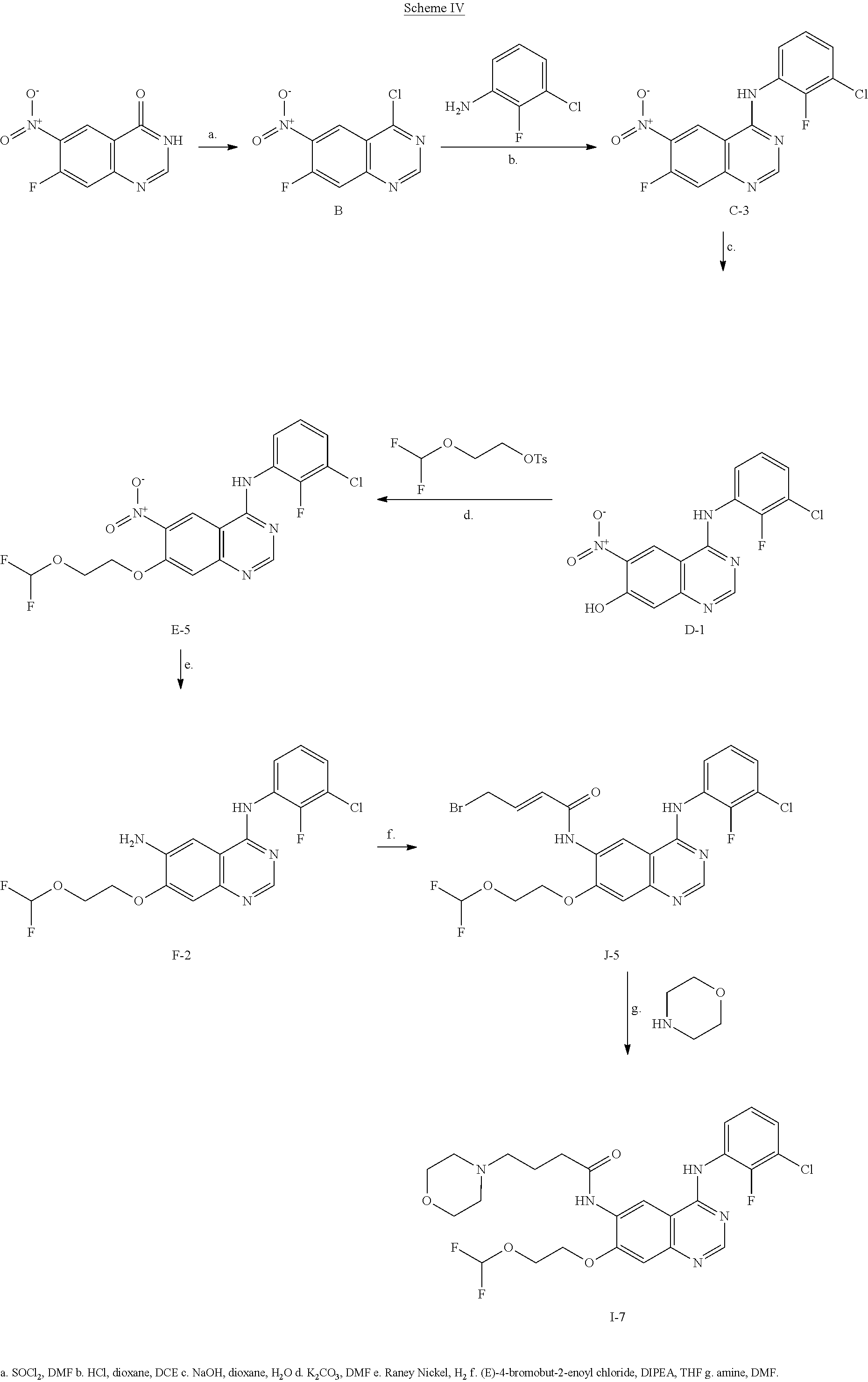

Scheme V outlines the synthesis of compounds of Formula I, represented by I-17, when R₁ is 3,4-dichloro-2-fluoroaniline group, R₂ is methoxy moiety and R₃ is methylene-morpholine.

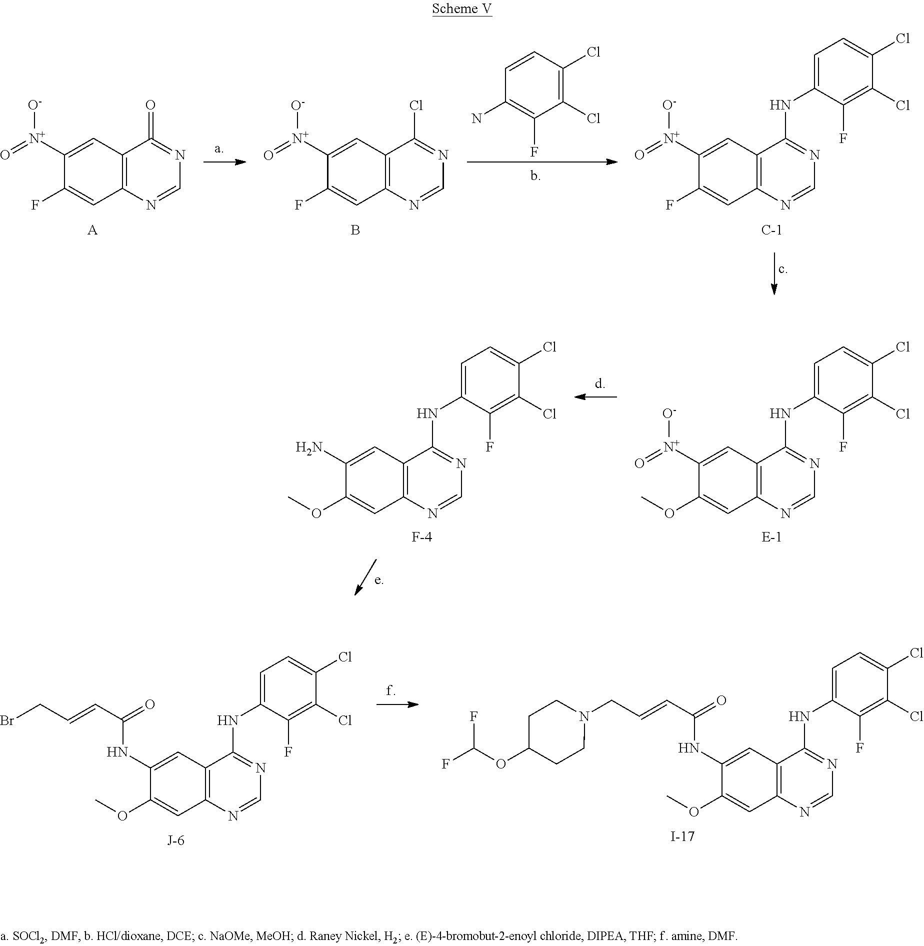

Scheme VI outlines the synthesis of compounds of Formula I, represented by I-25, when R₁ is 3-ethynyl-2-fluorophenyl) moiety, R₂ is 2-(difluoromethoxy)ethoxy) group and R₃ is dimethylamino.

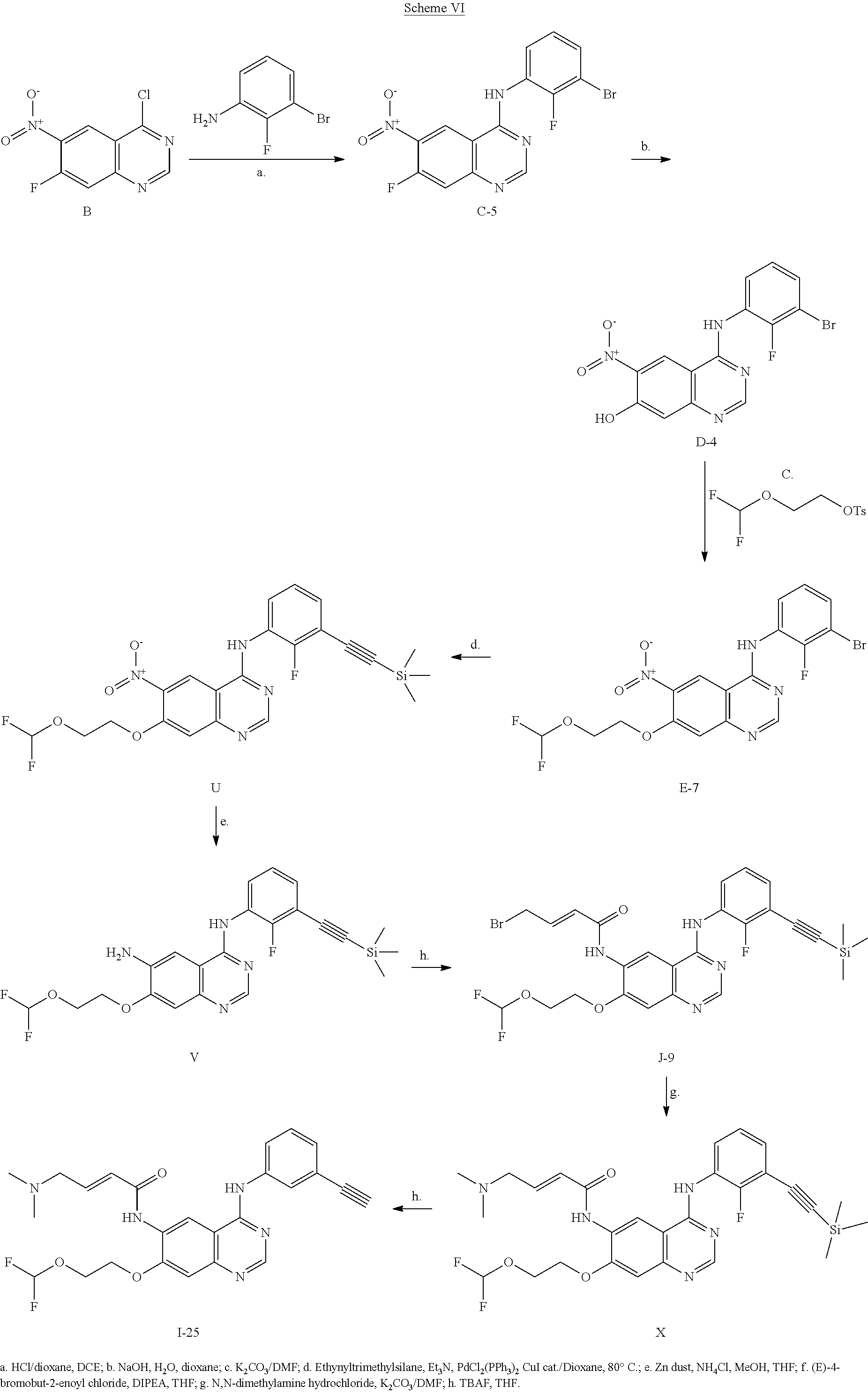

Scheme VII outlines the synthesis of (E)-4-bromobut-2-enoyl chloride intermediate (H).

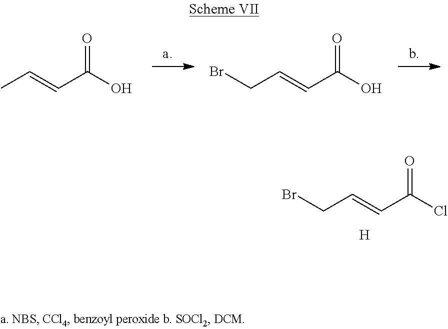

### Example 1: Preparation of 4-chloro-7-fluoro-6-nitro-quinazoline (B)

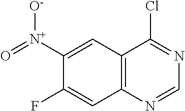 [SMILES: O=[N+]([O-])c1cc2c(Cl)ncnc2cc1F]

To a stirred solution of 7-fluoro-6-nitro-3H-quinazolin-4-one (Compound A in Scheme I, 3 g) in SOCl₂ (30 mL) was added DMF (1 drop). The resulting mixture was stirred at reflux temperature for 3 h. The mixture was then concentrated in vacuo giving the crude product as a pale yellow solid, used directly in the subsequent reaction.

### Example 2: Preparation of N-(3,4-dichloro-2-fluoro-phenyl)-7-fluoro-6-nitro-quinazolin-4-amine hydrochloride (Compound C-1)

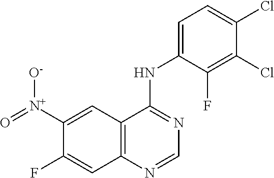 [SMILES: O=[N+]([O-])c1cc2c(Nc3ccc(Cl)c(Cl)c3F)ncnc2cc1F]

To a stirred suspension of 4-chloro-7-fluoro-6-nitro-quinazoline (Compounds B, 1.625 g, 7.14 mmol) in DCM (15 mL) was added 3,4-dichloro-2-fluoro-aniline (1.414 g, 7.85 mmol) as a solution in DCM (5 mL). The resulting mixture was stirred at room temperature overnight. The mixture was then diluted with diethyl ether and filtered to collect the desired product (2.35 g, 81%) as a pale solid.

¹H NMR (d6-DMSO, 300 MHz) δ 9.54 (d, J=9 Hz, 1H), 8.65 (s, 1H), 7.87 (d, J=12 Hz, 1H), 7.65-7.49 (m, 2H); MS: 371.4 (MH+).

In a similar manner, using the above procedure, the following N-aryl-7-fluoro-6-nitro-quinazolin-4-amines of Formula C were synthesized:

TABLE B

| Example | | | | Appearance |
|---|---|---|---|---|
| # | Structure | IUPAC Name | Analytical | (Yield %) |
| C-2 | 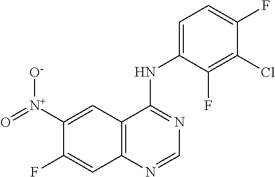 [SMILES: O=[N+]([O-])c1cc2c(Nc3ccc(F)c(Cl)c3F)ncnc2cc1F] | N-(3-chloro-2,4- difluorophenyl)-7- fluoro-6- nitroquinazolin-4- amine | NMR: (d6-DMSO, 300 MHz) δ 9.52 (d, J = 9 Hz, 1H), 8.65 (s, 1H), 7.86 (d, J = 12 Hz, 1H), 7.68-7.42 (m, 2H). | Pale yellow solid (95) |
| C-3 | 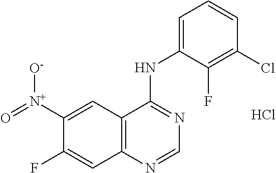 [SMILES: O=[N+]([O-])c1cc2c(Nc3cccc(Cl)c3F)ncnc2cc1F ; Cl] | N-(3-chloro-2- fluorophenyl)-7- fluoro-6- nitroquinazolin-4- amine hydrochloride | NMR: (d6-DMSO, 300 MHz) δ 9.60 (d, J = 6 Hz, 1H), 8.69 (s, 1H), 7.89 (d, J = 12 Hz, 1H), 7.61-7.46 (m, 2H), 7.32 (t, J = 9 Hz, 1H). | Pale yellow solid (Quant.) |
| C-4 | 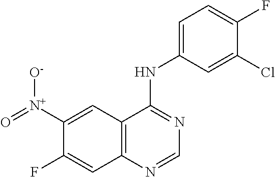 [SMILES: O=[N+]([O-])c1cc2c(Nc3ccc(F)c(Cl)c3)ncnc2cc1F] | N-(3-chloro-4- fluorophenyl)-7- fluoro-6- nitroquinazolin-4- amine | NMR: (d6-DMSO, 300 MHz) δ 10.90 (brs, 1H), 9.66 (d, J = 9 Hz, 1H), 8.79 (s, 1H), 8.11 (dd, J = 9, 3 Hz, 1H), 7.88 (d, J = 12 Hz, 1H), 7.82-7.75 (m, 1H), 7.50 (t, J = 9 Hz, 1H). MS: 337.0 (MH+). | Pale yellow solid (Quant.) |
| C-5 | 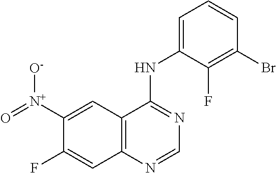 [SMILES: O=[N+]([O-])c1cc2c(Nc3cccc(Br)c3F)ncnc2cc1F] | N-(3-bromo-2- fluorophenyl)-7- fluoro-6- nitroquinazolin-4- amine hydrochloride | NMR: (d6-DMSO, 300 MHz) δ 9.70 (d, J = 9 Hz, 1H), 8.79 (brs, 1H), 8.77 (s, 1H), 7.93 (d, J = 12 Hz, 1H), 7.70 (t, J = 7.5 Hz, 1H), 7.54 (t, J = 9 Hz, 1H), 7.27 (t, J = 9 Hz, 1H). | Pale yellow solid (Quant.) |

### Example 3: Preparation of 4-(3-chloro-2-fluoro-anilino)-6-nitro-quinazolin-7-ol (D-1)

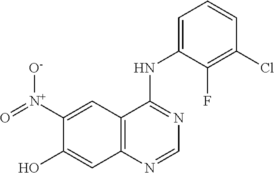 [SMILES: O=[N+]([O-])c1cc2c(Nc3cccc(Cl)c3F)ncnc2cc1O]

N-(3-Chloro-2-fluoro-phenyl)-7-fluoro-6-nitro-quinazolin-4-amine (Compound C-1, 5.6 g, 16.6 mmol) was stirred in dioxane (20 mL) and 50% NaOH (12 mL) at 110° C. for 5 h. The mixture was then cooled in an ice bath, acidified to pH 4 and stirred for 1 h. The mixture was then filtered to collect the solid material which was washed with water and diethyl ether. The filter cake was dried overnight in the oven to give the desired product as a red/orange solid (3.9 g). The mother liquor was extracted with ethyl acetate to obtain another 1.1 g (total yield 89%).

¹H NMR (d6-DMSO, 300 MHz) δ 9.40 (s, 1H), 8.80 (s, 1H), 7.70-7.61 (m, 1H), 7.61-7.47 (m, 2H), 7.36-7.28 (m, 1H). MS: 335.5 (MH+).

In a similar manner, using the above procedure, the following N-aryl-7-fluoro-6-nitro-quinazolin-4-amine were synthesized.

TABLE C

| Example | | | | Yield |
|---|---|---|---|---|
| # | Structure | Nomenclature | Appearance | (%) |
| D-2 | 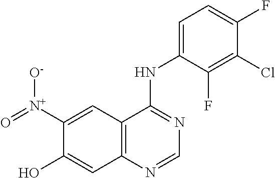 [SMILES: O=[N+]([O-])c1cc2c(Nc3ccc(F)c(Cl)c3F)ncnc2cc1O] | 4-((3-chloro-2,4-difluorophenyl)amino)- 6-nitroquinazolin-7-ol | Red-orange solid | 89 |
| | 1H NMR: (d6-DMSO, 400 MHz) δ 9.18 (s, 1H), 8.54 (s, 1H), 7.59-7.57 (s, 1H), 7.45-7.36 | | | |
| | (m, 1H), 7.30 (s, 1H). MS: 353.5 (MH+) | | | |
| D-3 | 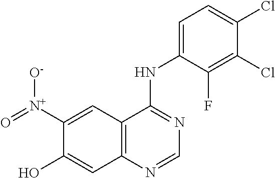 [SMILES: O=[N+]([O-])c1cc2c(Nc3ccc(Cl)c(Cl)c3F)ncnc2cc1O] | 4-((3,4-dichloro-2-fluorophenyl)amino)- 6-nitroquinazolin-7-ol | Reddish- yellow solid | 97 |
| | 1H NMR (D6-DMSO, 300 MHz) δ 9.40 (s, 1H), 8.80 (s, 1H), 7.70-7.61 (m, 1H), 7.61-7.50 | | | |
| | (m, 2H). MS: 369.0 (MH+) | | | |
| D-4 | 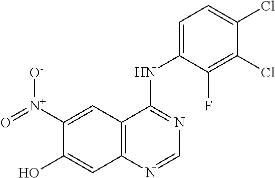 [SMILES: O=[N+]([O-])c1cc2c(Nc3ccc(Cl)c(Cl)c3F)ncnc2cc1O] | 4-((3-bromo-2-fluorophenyl)amino)-6- nilroquinazolin-7-ol | Yellow- orange solid | 82 |

1H NMR (d6-DMSO, 300 MHz) δ 11.88 (brs, 1H), 10.33 (brs, 1H), 9.13 (s, 1H), 8.45 (s, 1H), 7.60 (t, J = 7.5 Hz, 1H), 7.50 (t, J = 7.5 Hz, 1H), 7.24-7.16 (m, 2H).

### Example 4: Methanolysis of Fluorides

### Preparation of N-(3,4-dichloro-2-fluoro-phenyl)-7-methoxy-6-nitro-quinazolin-4-amine (E-1)

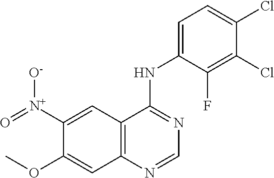 [SMILES: COc1cc2ncnc(Nc3ccc(Cl)c(Cl)c3F)c2cc1[N+](=O)[O-]]

To a stirred suspension of N-(3,4-dichloro-2-fluoro-phenyl)-7-fluoro-6-nitro-quinazolin-4-amine hydrochloride (Compound C-4, 2 g, 4.9 mmol) in methanol (20 mL) was added sodium methoxide (1.32 g, 24.5 mmol) and the resulting mixture was stirred at 70° C. After 1 h, more sodium methoxide (1.32 g, 24.5 mmol) was added and stirring was continued for 1 h. The mixture was cooled to room temperature and diluted with water then neutralized via addition of 3 M hydrochloric acid. The suspension was filtered to collect the desired product as a yellow solid (1.88 g, quantitative). 1H NMR (d6-DMSO, 300 MHz) δ 10.43 (brs, 1H), 9.12 (s, 1H), 8.53 (s, 1H), 7.61-7.49 (m, 2H), 7.47 (s, 1H), 4.05 (s, 3H).

In a similar manner, using the above procedure, the following N-aryl-7-methoxy-6-nitro-quinazolin-4-amine were synthesized.

TABLE D

| Example | | | | Apparence |
|---|---|---|---|---|
| # | Chemical Structure | IUPAC Name | MW | (Yield %) |
| E-2 | 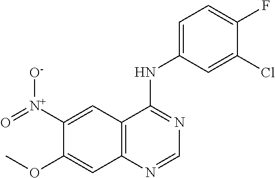 [SMILES: COc1cc2ncnc(Nc3ccc(F)c(Cl)c3)c2cc1[N+](=O)[O-]] | N-(3-chloro-4- fluoro-phenyl)- 7-methoxy-6- nitro- quinazolin-4- amine | 348.0 | Yellow solid (83%) |
| E-3 | 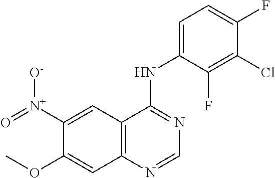 [SMILES: COc1cc2ncnc(Nc3ccc(F)c(Cl)c3F)c2cc1[N+](=O)[O-]] | N-(3-chloro- 2,4- difluorophenyl) 7-methoxy-6- nitroquinazolin 4-amine | 366.0 | Pale-yellow solid (100%) |

### Example 5: Preparation of N-(3,4-dichloro-2-fluoro-phenyl)-7-[2-(difluoromethoxy)ethoxy]-6-nitro-quinazolin-4-amine (E-4)

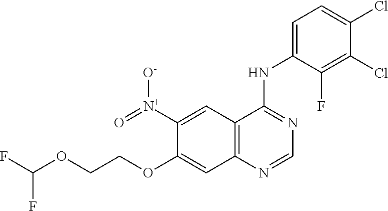 [SMILES: O=[N+]([O-])c1cc2c(Nc3ccc(Cl)c(Cl)c3F)ncnc2cc1OCCOC(F)F]

To a stirred solution of 4-(3,4-dichloro-2-fluoro-anilino)-6-nitro-quinazolin-7-ol (Compound D-2, 2.1 g, 5.69 mmol) was added potassium carbonate (2.35 g, 17.1 mmol) and 2-(difluoromethoxy)ethyl 4-methylbenzenesulfonate (2.27 g). The resulting mixture was stirred at 75° C. for 2 h then at room temperature overnight. The mixture was then diluted with ethyl acetate and washed with water and brine. The organic phase was dried, filtered and concentrated in vacuo then chromatographed in 30-40% ethyl acetate in hexanes. The product containing fractions were concentrated in vacuo giving the desired material as a yellow solid (1.15 g). NMR: (d6-DMSO, 300 MHz) δ 10.36 (s, 1H), 9.17 (s, 1H), 8.58 (s, 1H), 7.62-7.51 (m, 3H), 6.73 (t, J=76 Hz, 1H), 4.56-4.49 (m, 2H), 4.26-4.20 (m, 2H). MS: 433.21 (MH+).

In a similar manner, using the above procedure, the following N-aryl-7-[2-(difluoromethoxy)ethoxy]-6-nitro-quinazolin-4-amine were synthesized.

TABLE E

| Exam- | | | | Appear- |
|---|---|---|---|---|
| ple | | | | ence |
| # | Chemical Structure | IUPAC Name | Analytical | (Yield) |
| E-5 | 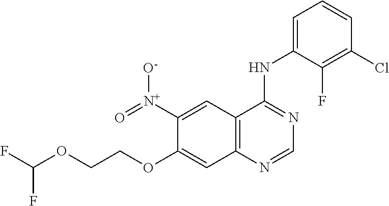 [SMILES: O=[N+]([O-])c1cc2c(Nc3cccc(Cl)c3F)ncnc2cc1OCCOC(F)F] | N-(3-chloro-2- fluorophenyl)- 7-(2- (difluoromethoxy) ethoxy)-6- nitroquinazolin-4-amine | NMR: (d6-DMSO, 300 MHz) δ 10.34 (s, 1H), 9.17 (s, 1H), 8.57 (s, 1H), 7.62-7.51 (m, 4H), 6.73 (t, J = 76 Hz, 1H), 4.57- 4.48 (m, 2H), 4.27-4.19 (m, 2H). MS: 428.15 (MH+). | Yellow solid (40%) |
| E-6 | 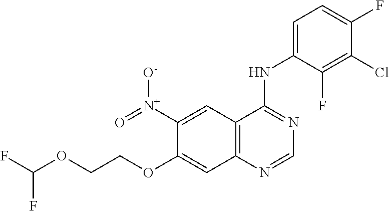 [SMILES: O=[N+]([O-])c1cc2c(Nc3ccc(F)c(Cl)c3F)ncnc2cc1OCCOC(F)F] | N-(3-chloro-2,4- difluorophenyl)-7-(2- (difluoromethoxy) ethoxy)-6- nitroquinazolin-4-amine | NMR: (d6-DMSO, 300 MHz) δ 10.31 (s, 1H), 9.16 (s, 1H), 8.56 (s, 1H), 7.62-7.51 (m, 1H), 7.54 (s, 1H), 7.45-7.34 (m, 1H), 6.73 (t, J = 76 Hz, 1H), 4.58- 4.50 (m, 2H), 4.27-4.19 (m, 2H). MS: 447.5 (MH+). | Yellow/ orange (40%) |
| E-7 | 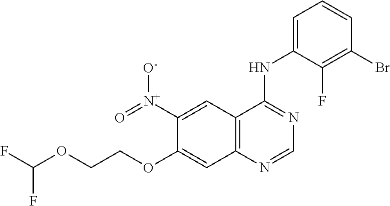 [SMILES: O=[N+]([O-])c1cc2c(Nc3cccc(Br)c3F)ncnc2cc1OCCOC(F)F] | N-(3-bromo-2- fluorophenyl)-7-(2- (difluoromethoxy) ethoxy)-6- nitroquinazolin-4-amine | NMR: (d6-DMSO, 300 MHz) δ 10.31 (s, 1H), 9.16 (s, 1H), 8.56 (s, 1H), 7.61-7.51 (m, 1H), 7.54 (s, 1H), 7.45-7.35 (m, 1H), 7.25-7.01 (m, 1H), 6.73 (t, J = 76 Hz, 1H), 4.55- 4.50 (m, 2H), 4.25-4.19 (m, 2H). | Yellow solid (45) |

### Example 6: Preparation of N4-(3,4-dichloro-2-fluoro-phenyl)-7-[2 (difluoromethoxy)ethoxy]quinazoline-4,6-diamine (F-1)

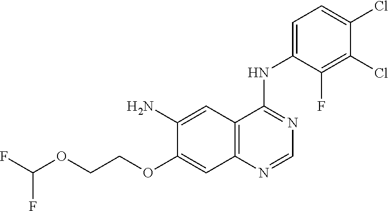 [SMILES: Nc1cc2c(Nc3ccc(Cl)c(Cl)c3F)ncnc2cc1OCCOC(F)F]

N-(3,4-dichloro-2-fluoro-phenyl)-7-[2-(difluoromethoxy)ethoxy]-6-nitro-quinazolin-4-amine (Compound E-4, 1.15 g) and Raney nickel (1.1 g) was stirred in THF (20 mL) under an atmosphere of hydrogen (balloon pressure) for 2 d. The mixture was then filtered and concentrated in vacuo then triturated with diethyl ether and hexanes. The resulting suspension was filtered to collect the desired product (0.66 g, 62%) as a tan solid. NMR: (d6-DMSO, 300 MHz) δ 9.52 (brs, 1H), 8.28 (s, 1H), 7.64-7.48 (m, 2H), 7.32 (s, 1H), 7.12 (s, 1H), 6.82 (t, J=75 Hz, 1H), 5.45 (brs, 2H), 4.40-4.32 (m, 2H), 4.32-4.24 (m, 2H). 398.77 (MH+).

In a similar manner, using the above procedure, the following N-aryl-7-[2-(difluoromethoxy)ethoxy]-6-nitro-quinazolin-4,6-diamine intermediates were synthesized.

TABLE F

| Exam- | | | | |
|---|---|---|---|---|
| ple | | | | Yield |
| # | Structure | Nomenclature | Appearance | (%) |
| F-2 | 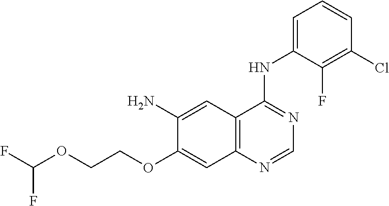 [SMILES: Nc1cc2c(Nc3cccc(Cl)c3F)ncnc2cc1OCCOC(F)F] | N4-(3-chloro-2-fluoro-phenyl)-7-[2- (difluoromethoxy)ethoxy]quinazoline- 4,6-diamine | Beige solid | 400 mg, 42% |
| | 1H NMR: (d6-DMSO, 300 MHz) 9.31 (s, 1H), 8.23 (s, 1H), 7.56-7.48 (m, 1H), 7.43-7.36 | | | |
| | (m, 1H), 7.32 (s, 1H), 7.22 (t, J = 8 Hz, 1H), 7.11 (s, 1H), 6.82 (t, J = 75 Hz, 1H), 5.36 (s, | | | |
| | 1H), 4.39-4.33 (m, 2H), 4.32-4.25 (m, 2H). | | | |
| F-3 | 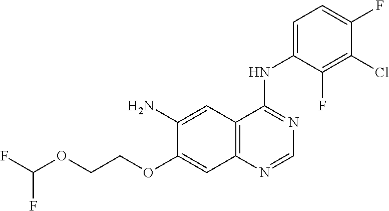 [SMILES: Nc1cc2c(Nc3ccc(F)c(Cl)c3F)ncnc2cc1OCCOC(F)F] | N4-(3-chloro-2,4-difluoro- phenyl)-7-[2- (difluoromethoxy)ethoxy]quinazoline- 4,6-diamine | White solid | 385 mg, 41% |
| | 1H NMR: (d6-DMSO, 300 MHz) δ 9.33 (s, 1H), 8.20 (s, 1H), 7.58-7.46 (m, 1H), 7.39-7.29 | | | |
| | (m, 1H), 7.29 (s, 1H), 7.10 (s, 1H), 6.80 (t, J = 76 Hz, 1H), 5.36 (s, 2H), 4.39-4.31 (m, | | | |
| | 2H), 4.31-4.23 (m, 2H). MS: 398.77 (MH+) | | | |
| F-4 | 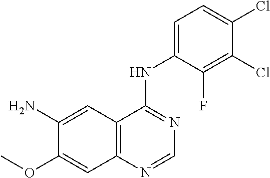 [SMILES: COc1cc2ncnc(Nc3ccc(Cl)c(Cl)c3F)c2cc1N] | N4-(3,4-dichloro-2-fluoro-phenyl)-7- methoxy-quinazoline-4,6-diamine | Green-grey powder | 320 mg, 18% |
| | 1H NMR: (d6-DMSO, 300 MHz) δ 9.35 (s, 1H), 8.24 (s, 1H), 7.64-7.55 (m, 1H), 7.55-7.48 | | | |
| | (m, 1H), 7.27 (s, 1H), 7.09 (s, 1H), 5.41 (s, 2H), 3.95 (s, 3H). MS: 353.4 (MH+) | | | |
| F-5 | 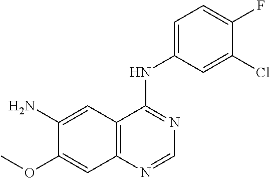 [SMILES: COc1cc2ncnc(Nc3ccc(F)c(Cl)c3)c2cc1N] | N4-(3-chloro-4-fluoro-phenyl)-7- methoxy-quinazoline-4,6-diamine | Beige solid | 948 mg, 94% |
| | 1H NMR: (d6-DMSO, 300 MHz) δ 9.36 (s, 1H), 8.36 (s, 1H), 8.17 (dd, J = 9, 3 Hz, 1H), 7.82-7.75 (m, 1H), 7.41-7.34 (m, 2H), 7.09 (s, 1H), 5.35 (s, 2H), 3.95 (s, 3H). | | | |
| F-6 | 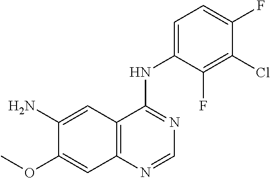 [SMILES: COc1cc2ncnc(Nc3ccc(F)c(Cl)c3F)c2cc1N] | N4-(3-chloro-2,4-difluoro-phenyl)-7- methoxy-quinazoline-4,6-diamine | tan solid | 3.36 g, 96% |

1H NMR: (d6-DMSO, 300 MHz) δ 9.30 (s, 1H), 8.21 (s, 1H), 7.58-7.47 (m, 1H), 7.38-7.29
(m, 1H), 7.27 (s, 1H), 7.08 (s, 1H), 5.38 (s, 2H), 3.95 (s, 3H). MS: 337.5 (MH+).

### Example 7: Preparation of (E)-4-bromobut-2-enoyl chloride

### (a) (E)-4-bromobut-2-enoic acid

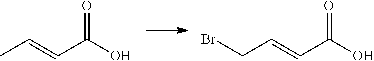

To a stirred suspension of crotonic acid (10 g, 116 mmoL) and N-bromosuccinimide (21.3 g, 119 mmol) in carbon tetrachloride (100 mL) was added benzoyl peroxide (197 mg, 8.13 mmol) and the resulting mixture was stirred at reflux temperature for 4 h. The mixture was cooled to room temperature and filtered. The filtrate was concentrated in vacuo then chromatographed in 0-20% ethyl acetate in hexanes. The product containing fractions were concentrated in vacuo then triturated in hexanes giving the desired product as a white solid (5 g, 26%).

### (b) (E)-4-bromobut-2-enoyl chloride

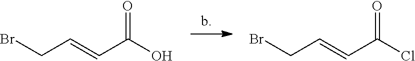 [SMILES: O=C(O)/C=C/CBr ; O=C(Cl)/C=C/CBr]

To a stirred solution of (E)-4-bromobut-2-enoic acid (214 mg, 1.295 mmol) in DCM (8 mL) was added (COCl)₂ (493.5 mg, 3.89 mmol) followed by a drop of DMF. The resulting mixture was stirred at RT until bubbling ceased and concentrated in vacuo. The crude material was used directly in the subsequent reaction.

### Example 8: Preparation of (E)-4-bromo-N-[4-(3,4-dichloro-2-fluoro-anilino)-7-[2-(difluoromethoxy)ethoxy]quinazolin-6-yl]but-2-enamide (J-1)

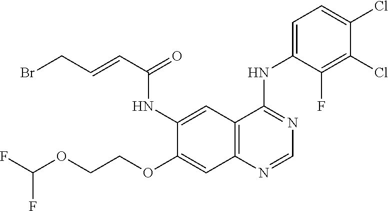 [SMILES: O=C(/C=C/CBr)Nc1cc2c(Nc3ccc(Cl)c(Cl)c3F)ncnc2cc1OCCOC(F)F]

To a stirred suspension of N4-(3,4-dichloro-2-fluoro-phenyl)-7-[2-(difluoromethoxy)ethoxy]quinazoline-4,6-diamine (217 mg, 0.5 mmol) and DIPEA (96.7 mg, 0.75 mmol) in THF (8 mL) cooled to −78° C. was added (E)-4-bromobut-2-enoyl chloride (138 mg, 0.75 mmol) as a solution in DCM (1 mL). The resulting mixture was stirred at 0° C. for 2 h. The mixture was diluted with ethyl acetate and washed with water and brine. The organic phase was dried, filtered and concentrated then purified via column chromatography to give the desired product (190 mg, 66%) as a white solid. NMR: (d6-DMSO, 300 MHz) δ 9.95 (s, 1H), 9.63 (s, 1H), 8.92 (s, 1H), 8.43 (s, 1H), 7.60-7.47 (m, 2H), 7.38-7.29 (m, 1H), 6.99-6.83 (m, 1H), 6.79 (t, J=76 Hz, 1H), 6.66 (d, J=15 Hz, 1H), 4.50-4.39 (m, 2H), 4.39-4.20 (m, 4H). MS: 577.97 (MH+).

In a similar manner, using the above procedure, the following (E)-4-bromo-N-[aryl-7)-7-[2-(difluoromethoxy)ethoxy] and methoxy-quinazolin-6-yl]but-2-enamide intermediates were synthesized.

TABLE G

| Exam- | | | | |
|---|---|---|---|---|
| ple | | | | Yield |
| # | Structure | Nomenclature | Appearance | (%) |
| J-2 | 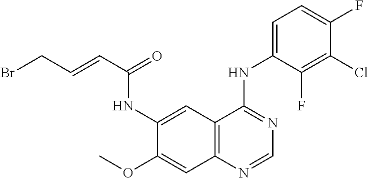 [SMILES: COc1cc2ncnc(Nc3ccc(F)c(Cl)c3F)c2cc1NC(=O)/C=C/CBr] | (E)-4-bromo-N-(4-((3-chloro-2,4- difluoro-phenyl)-amino)-7-methoxy quinazolin-6-yl)but-2-enamide | Pale solid | 55% |
| | 1H NMR: (d6-DMSO, 300 MHz) δ 9.91 (brs, 1H), 9.85 (s, 1H), 8.92 (s, 1H), 8.40 (s, 1H), 7.56-7.42 (m, | | | |
| | 1H), 7.43-7.30 (m, 1H), 7.27 (s, 1H), 6.98-6.83 (m, 1H), 6.79-6.63 (m, 1H), 4.44-4.28 (m, 1H), 4.00 (s, | | | |
| | 3H). MS: 485.5 (MH+) | | | |
| J-3 | 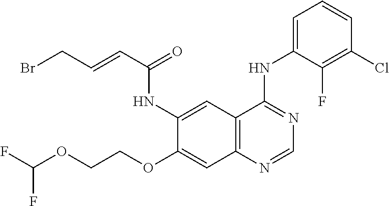 [SMILES: O=C(/C=C/CBr)Nc1cc2c(Nc3cccc(Cl)c3F)ncnc2cc1OCCOC(F)F] | E)-4-bromo-N-(4-((3-chloro-2- fluorophenyl)amino)-7-(2- (difluoromethoxy)ethoxy) quinazolin-6-yl)but-2-enamide | Pale solid | 86% |
| | 1H NMR: (d6-DMSO, 300 MHz) δ 9.89 (s, 1H), 9.64 (s, 1H), 8.91 (s, 1H), 8.41 (s, 1H), 7.52-7.39 (m, 2H), | | | |
| | 7.32 (s, 1H), 7.30-7.20 (m, 1H), 6.99-6.84 (m, 1H), 6.78 (t, J = 75 Hz, 1H), 6.64 (d, J = 15 Hz, 1H), 4.50- | | | |
| | 4.38 (m, 2H), 4.36-4.25 (m, 2H), 3.33-3.19 (m, 2H). | | | |
| J-4 | 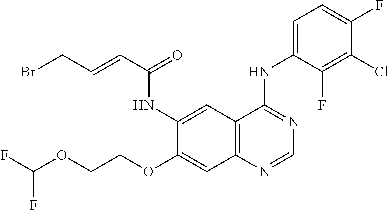 [SMILES: O=C(/C=C/CBr)Nc1cc2c(Nc3ccc(F)c(Cl)c3F)ncnc2cc1OCCOC(F)F] | (E)-4-bromo-N-(4-((3-chloro-2,4- difluorophenyl)amino)-7-(2- (difluoromethoxy)ethoxy) quinazolin-6-yl)but-2-enamide | Pale solid | 63% |
| | 1H NMR: (d6-DMSO, 300 MHz) δ 9.84 (s, 1H), 9.42 (s, 1H), 8.93 (s, 1H), 8.39 (s, 1H), 7.55-7.54 (m, 1H), | | | |
| | 7.41-7.31 (m, 1H), 7.31 (s, 1H), 6.85-6.73 (m, 1H), 6.80 (t, J = 75 Hz, 1H), 6.52 (d, J = 15 Hz, 1H), 4.48- | | | |
| | 4.40 (m, 2H), 4.35-4.21 (m, 4H). | | | |
| J-5 | 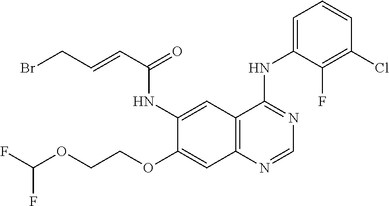 [SMILES: O=C(/C=C/CBr)Nc1cc2c(Nc3cccc(Cl)c3F)ncnc2cc1OCCOC(F)F] | (E)-4-bromo-N-(4-((3-chloro-2- fluorophenyl)amino)-7-(2- (difluoromethoxy)ethoxy)quinazolin- 6-yl)but-2-enamide | Pale solid | |
| | 1H NMR: (d6-DMSO, 300 MHz) δ 9.90 (s, 1H), 9.64 (s, 1H), 8.90 (s, 1H), 8.40 (s, 1H), 7.51-7.41 (m, 2H), | | | |
| | 7.32 (s, 1H), 7.30-7.20 (m, 1H), 6.99-6.85 (m, 1H), 6.78 (t, J = 76 Hz, 1H), 6.64 (d. J = 15 Hz, 1H), 4.48- | | | |
| | 4.39 (m, 2H), 4.36-4.26 (m, 4H). | | | |
| J-6 | 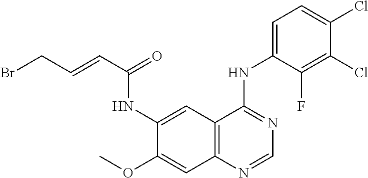 [SMILES: COc1cc2ncnc(Nc3ccc(Cl)c(Cl)c3F)c2cc1NC(=O)/C=C/CBr] | (E)-4-bromo-N-(4-((3,4-dichloro-2- fluorophenyl)amino)-7- methoxyquinazolin-6-yl)but-2-enamide | Pale solid | 76% |
| | 1H NMR: (d6-DMSO, 300 MHz) δ 9.90 (s, 1H), 9.66 (s, 1H), 8.94 (s, 1H), 8.41 (s, 1H), 7.58-7.49 (m, 2H), | | | |
| | 7.28 (s, 1H), 6.86-6.66 (m, 1H), 6.60 (d, J = 15 Hz, 1H), 4.01 (s, 3H), 4.35-4.28 (m, 2H). MS: 577.97 (MH+). | | | |
| J-7 | 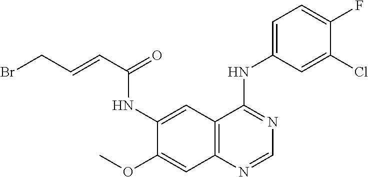 [SMILES: COc1cc2ncnc(Nc3ccc(F)c(Cl)c3)c2cc1NC(=O)/C=C/CBr] | (E)-4-bromo-N-(4-((3-chloro-4- fluorophenyl)amino)-7- methoxyquinazolin-6-yl)but-2-enamide | beige solid | 73% |
| | 1H NMR: (d6-DMSO, 300 MHz) δ 9.87-9.67 (m, 2H), 8.93-8.77 (m, 1H), 8.52 (s, 1H), 8.15-8.06 (m, 1H), | | | |
| | 7.83-7.73 (m, 1H), 7.46-7.35 (t, J = 9 Hz, 1H), 7.30-7.25 (m, 1H), 6.68-6.38 (m, 2H), 4.00 (s, 3H), 3.51- | | | |
| | 3.35 (m, 2H). | | | |
| J-8 | 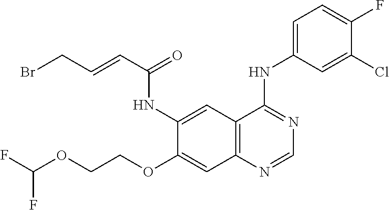 [SMILES: O=C(/C=C/CBr)Nc1cc2c(Nc3ccc(F)c(Cl)c3)ncnc2cc1OCCOC(F)F] | (E)-4-bromo-N-(4-((3-chloro-4- fluorophenyl)amino)-7-(2- (difluoromethoxy)ethoxy)quinazolin- 6-yl)but-2-enamide | tan solid | 47% |
| | 1H NMR: (d6-DMSO, 300 MHz) δ 9.81 (s, 1H), 9.47 (s, 1H), 8.78 (s, 1H), 8.53 (s, 1H), 8.15-8.07 (m, 1H), | | | |
| | 7.83-7.72 (m, 1H), 7.40 (t, J = 9 Hz, 1H), 7.32 (s, 1H), 6.80 (t, J = 75 Hz, 1H), 6.67-6.45 (m, 2H), 4.49- | | | |
| | 4.38 (m, 2H), 4.37-4.23 (m, 4H). MS 545.5 (MH+) | | | |
| J-9 | 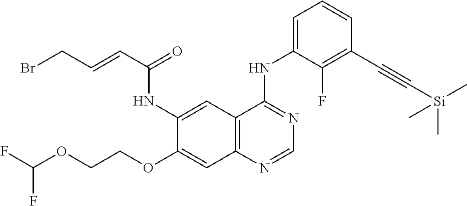 [SMILES: C[Si](C)(C)C#Cc1cccc(Nc2ncnc3cc(OCCOC(F)F)c(NC(=O)/C=C/CBr)cc23)c1F] | (E)-4-bromo-N-(7-(2-(difluoromethoxy) ethoxy)-4-((2-fluoro-3-((trimethylsilyl) ethynyl)phenyl)amino)quinazolin- 6-yl)but-2-enamide | Beige solid | 97% |

MS:607

### Example 9: Preparation of N-[4-(3,4-dichloro-2-fluoro-anilino)-7-[2-(difluoromethoxy)ethoxy]quinazolin-6-yl]prop-2-enamide (Compound I-1)

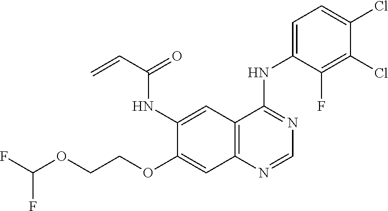 [SMILES: C=CC(=O)Nc1cc2c(Nc3ccc(Cl)c(Cl)c3F)ncnc2cc1OCCOC(F)F]

To a stirred solution N4-(3,4-dichloro-2-fluoro-phenyl)-7-[2-(difluoromethoxy)ethoxy]quinazoline-4,6-diamine (100 mg, 0.231 mmol) in THF (5 mL) cooled to 0° C. was added DIPEA, followed by acryloyl chloride (31.3 mg, 0.346 mmol) as a solution in DCM (1 mL). The resulting mixture was stirred at 0° C. for 3.5 h. The mixture was then diluted with ethyl acetate and washed with water (1×), sat. NaHCO₃ (1×) and brine (1×). The organic phase was dried, filtered and concentrated then chromatographed in 40-80% ethyl acetate in hexanes giving the desired product, N-[4-(3,4-dichloro-2-fluoro-anilino)-7-[2-(difluoromethoxy)ethoxy]quinazolin-6-yl]prop-2-enamide, as a pale yellow to white solid (20-45 mg, 18-40%). ¹H NMR (d6-DMSO, 300 MHz) δ 9.94 (s, 1H), 9.53 (s, 1H), 8.94 (s, 1H), 8.42 (s, 1H), 7.58-7.49 (m, 2H), 7.34 (s, 1H), 6.79 (t, J=75 Hz, 1H), 6.69 (dd, J=17, 10 Hz, 1H), 6.30 (dd, J=18, 3 Hz, 1H), 5.82 (dd, J=10, 3 Hz, 1H), 4.48-4.42 (m, 2H), 4.34-4.27 (m, 2H). MS (MH+):488.5

### Example 10: Preparation of (E)-N-[4-(3-chloro-2-fluoro-anilino)-7-[2-(difluoromethoxy)ethoxy]quinazolin-6-yl]-4-(dimethylamino)but-2-enamide (Compounds I-5)

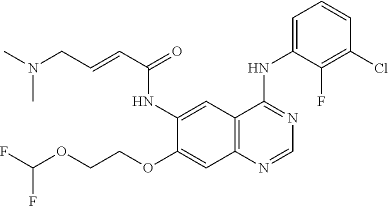 [SMILES: CN(C)C/C=C/C(=O)Nc1cc2c(Nc3cccc(Cl)c3F)ncnc2cc1OCCOC(F)F]

To a stirred solution of (E)-4-bromo-N-[4-(3-chloro-2-fluoro-anilino)-7-[2-(difluoromethoxy)ethoxy]quinazolin-6-yl]but-2-enamide (54.3 mg, 0.1 mmol) in DMF (3 mL) was added dimethylamine (2M in THF, 0.25 mL, 0.5 mmol) and the resulting mixture was stirred overnight at room temperature. The mixture was then diluted with EtOAc and washed with NaHCO₃ (2×) and brine (1×). The organic phase was dried, filtered and concentrated then stirred in diethyl ether. The resulting suspension was filtered to collect the desired product (64-80%) as a white solid. ¹H NMR (d6-DMSO, 400 MHz) δ 9.86 (s, 1H), 9.42 (s, 1H), 8.93 (s, 1H), 8.40 (s, 1H), 7.52-7.39 (m, 2H), 7.32 (s, 1H), 7.29-7.20 (m, 1H), 6.84-6.74 (m, 1H), 6.79 (t, J=78 Hz, 1H), 6.52 (d, J=12 Hz, 1H), 4.46-4.41 (m, 2H), 4.34-4.27 (m, 2H), 3.08 (d, J=4 Hz, 2H), 2.17 (s, 6H). MS (MH+):510.7

In a similar manner, using the above procedure, the following (E)-N-aryl)-7-[2-(difluoromethoxy)ethoxy] or methoxy-quinazolin-6-yl]-4-(dimethylamino)-but-2-enamide derivatives were synthesized.

TABLE H

| Exam- | | | | |
|---|---|---|---|---|
| ple | | | Appear- | Yield |
| # | Chemical Structure | UPAC Nomenclature | ance | (%) |
| I-2 | 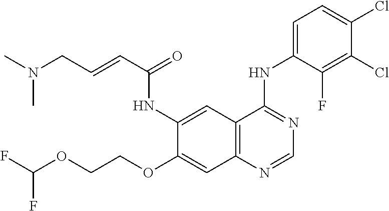 [SMILES: CN(C)C/C=C/C(=O)Nc1cc2c(Nc3ccc(Cl)c(Cl)c3F)ncnc2cc1OCCOC(F)F] | (E)-N-[4-(3,4-dichloro-2- fluoro-anilino)-7-[2- (difluoromethoxy)ethoxy] quinazolin-6-yl]-4- (dimethylamino)but-2- enamide | White solid | 42 |
| | ¹H NMR (d6-DMSO, 300 MHz) δ 9.92 (s, 1H), 9.45 (s, 1H), 8.93 (s, 1H), 8.41 (s, 1H), | | | |
| | 7.57-7.51 (m, 2H), 7.32 (s, 1H), 6.84-6.73 (m, 1H), 6.78 (t, J = 75 Hz, 1H), 6.56-6.47 | | | |
| | (m, 1H), 4.47-4.40 (m, 2H), 4.34-4.37 (m, 2H), 3.07 (d, J = 6 Hz, 2H), 2.16 (s, 6H). | | | |
| | MS (MH+): 544.4 | | | |
| I-3 | 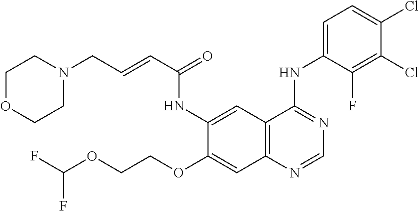 [SMILES: O=C(/C=C/CN1CCOCC1)Nc1cc2c(Nc3ccc(Cl)c(Cl)c3F)ncnc2cc1OCCOC(F)F] | (E)-N-[4-(3,4-dichloro-2- fluoro-anilino)-7-[2- (difluoromethoxy)ethoxy] quinazolin-6-yl]-4- morpholino-but-2- enamide | White solid | 73 |
| | ¹H NMR (d6-DMSO, 300 MHz) δ 9.93 (brs, 1H), 9.44 (s, 1H), 8.92 (s, 1H), 8.40 (s, | | | |
| | 1H), 7.57-7.49 (m, 2H), 7.31 (s, 1H), 7.84-6.73 (m, 1H), 6.78 (t, J = 75 Hz, 1H), 6.52 | | | |
| | (d, J = 15 Hz, 1H), 4.47-4.40 (m, 2H), 4.34-4.27 (m, 2H), 3.62-3.55 (m, 4H), 3.14 (d, | | | |
| | J = 6 Hz, 2H), 2.42-2.34 (m, 4H). MS (MH+): 586.6 | | | |
| I-4 | 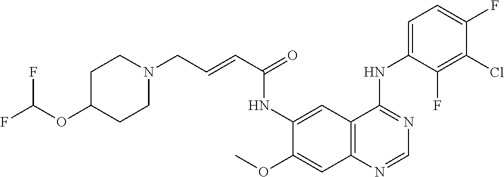 [SMILES: COc1cc2ncnc(Nc3ccc(F)c(Cl)c3F)c2cc1NC(=O)/C=C/CN1CCC(OC(F)F)CC1] | (E)-N-[4-(3-chloro-2,4- difluoro-anilino)-7- methoxy-quinazolin-6- yl]-4-[4- (difluoromethoxy)-1- piperidyl]but-2-enamide | White solid | 37 |
| | ¹H NMR (d6-DMSO, 300 MHz) δ 9.83 (s, 1H), 9.65 (s, 1H), 8.92 (s, 1H), 8.39 (s, 1H), | | | |
| | 7.55-7.44 (m, 1H), 7.40-7.30 (m, 1H), 7.27 (s, 1H), 6.83-6.71 (m, 1H), 6.71 (t, J = 75 | | | |
| | Hz, 1H), 6.58 (d, J = 15 Hz, 1H), 4.17-4.06 (m, 1H), 4.01 (s, 3H), 3.13 (d, J = 6 Hz, | | | |
| | 2H), 2.72-2.61 (m, 2H), 2.25-2.13 (m, 2H), 1.93-1.80 (m, 2H), 1.71-1.56 (m, 2H). | | | |
| | MS (MH+): 554.7 | | | |
| I-6 | 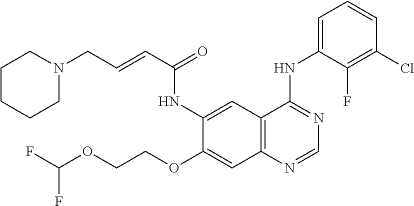 [SMILES: O=C(/C=C/CN1CCCCC1)Nc1cc2c(Nc3cccc(Cl)c3F)ncnc2cc1OCCOC(F)F] | (E)-N-[4-(3-chloro-2- fluoro-anilino)-7-[2- (difluoromethoxy)ethoxy] quinazolin-6-yl]-4-(1- piperidyl)but-2-enamide | White solid | 46 |
| | ¹H NMR (d6-DMSO, 400 MHz) δ 9.86 (s, 1H), 9.41 (s, 1H), 8.92 (s, 1H), 8.40 (s, 1H), | | | |
| | 7.50-7.42 (m, 2H), 7.32 (s, 1H), 7.27-7.21 (m, 1H), 6.84-6.75 (m, 1H), 6.79 (t, J = 76 | | | |
| | Hz, 1H), 6.49 (d, J = 16 Hz, 1H), 4.46-4.41 (m, 2H), 4.33-4.28 (m, 2H), 3.09 (d, J = 8 | | | |
| | Hz, 12H), 2.38-2.29 (m, 4H), 1.54-1.47 (m, 4H), 1.42-1.33 (m, 2H). MS (MH+): 550.8 | | | |
| I-7 | 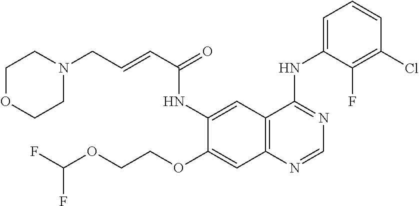 [SMILES: O=C(/C=C/CN1CCOCC1)Nc1cc2c(Nc3cccc(Cl)c3F)ncnc2cc1OCCOC(F)F] | (E)-N-[4-(3-chloro-2- fluoro-anilino)-7-[2- (difluoromethoxy)ethoxy] quinazolin-6-yl]-4- morpholino-but-2- enamide | White solid | 55 |
| | ¹H NMR (d6-DMSO, 400 MHz) δ 9.86 (s, 1H), 9.43 (s,1H), 8.92 (s, 1H), 8.40 (s, 1H), | | | |
| | 7.50-7.40 (m, 2H), 7.32 (s, 1H), 7.27-7.21 (m, 1H), 6.82-6.74 (m, 1H), 6.80 (t, J = 76 | | | |
| | Hz, 1H), 6.53 (d, J = 16 Hz, 1H), 4.47-4.41 (m, 2H), 4.33-4.27 (m, 2H), 3.62-3.54 (m, | | | |
| | 4H), 3.14 (d, J = 8 Hz, 2H), 2.42-2.34 (m, 4H). MS (MH+): 552.6 | | | |
| I-8 | 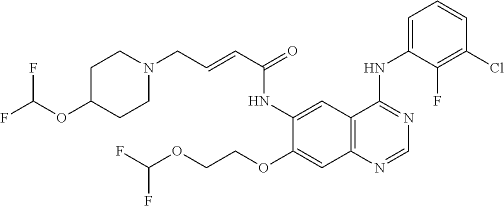 [SMILES: O=C(/C=C/CN1CCC(OC(F)F)CC1)Nc1cc2c(Nc3cccc(Cl)c3F)ncnc2cc1OCCOC(F)F] | (E)-N-[4-(3-chloro-2- fluoro-anilino)-7-[2- (difluoromethoxy)ethoxy] quinazolin-6-yl]-4-[4- (difluoromethoxy)-l- piperidyl]but-2-enamide | White solid | 48 |
| | ¹H NMR (d6-DMSO, 400 MHz) δ 9.86 (s, 1H), 9.42 (s, 1H), 8.92 (s, 1H), 8.40 (s, 1H), | | | |
| | 7.50-7.41 (m, 2H), 7.32 (s, 1H), 7.27-7.20 (m, 1H), 6.84-6.75 (m, 1H), 6.80 (t, J = 76 | | | |
| | Hz, 1H), 6.70 (t, J = 76 Hz, 1H), 6.50 (d, J = 16 Hz, 1H), 4.47-4.40 (m, 2H), 4.33-4.27 | | | |
| | (m, 2H), 4.17-4.06 (m, 1H), 3.14 (d, J = 8 Hz, 12H), 2.72-2.61(m, 2H), 2.25-2.13 (m, | | | |
| | 2H), 1.92-1.80 (m, 2H), 1.68-1.54 (m, 2H). MS (MH+): 616.7 | | | |
| I-9 | 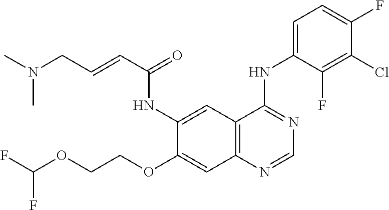 [SMILES: CN(C)C/C=C/C(=O)Nc1cc2c(Nc3ccc(F)c(Cl)c3F)ncnc2cc1OCCOC(F)F] | (E)-N-[4-(3-chloro-2,4- difluoro-anilino)-7-[2- (difluoromethoxy)ethoxy] quinazolin-6-yl]-4- (dimethylamino)but-2- enamide | White solid | 60 |
| | ¹H NMR (d6-DMSO, 300 MHz) δ 9.86 (s, 1H), 9.43 (s, 1H), 8.93 (s, 1H), 8.39 (s, 1H), | | | |
| | 7.55-7.54 (m, 1H), 7.41-7.31 (m, 1H), 7.32 (s, 1H), 6.85-6.73 (m, 1H), 6.79 (t, J = 75 | | | |
| | Hz, 1H), 6.52 (d, = 15 Hz, 1H), 4.47-4.40 (m, 2H), 4.34-4.26 (m, 2H), 3.08 (d, J = 6 Hz, | | | |
| | 2H), 2.17 (s, 6H). MS (MH+): 528.5 | | | |
| I-10 | 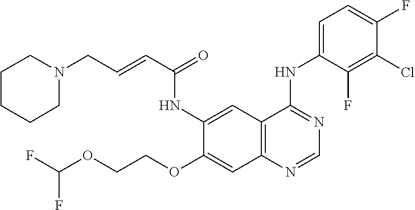 [SMILES: O=C(/C=C/CN1CCCCC1)Nc1cc2c(Nc3ccc(F)c(Cl)c3F)ncnc2cc1OCCOC(F)F] | (E)-N-[4-(3-chloro-2,4- difluoro-anilino)-7-[2- (difluoromethoxy)ethoxy] quinazolin-6-yl]-4-(1- piperidyl)but-2-enamide | White solid | 53 |
| | ¹H NMR (d6-DMSO, 300 MHz) δ 9.86 (s, 1H), 9.41 (s, 1H), 8.92 (s, 1H), 8.39 (s, 1H), | | | |
| | 7.56-7.45 (m, 1H), 7.40-7.31 (m, 1H), 7.32 (s, 1H), 6.85-6.73 (m, 1H), 6.80 (t, J = 76 | | | |
| | Hz, 1H), 6.50 (d, J = 15 Hz, 1H), 4.48-4.40 (m, 2H), 4.35-4.27 (m, 2H), 3.10 (d, J = 9 | | | |
| | Hz, 2H), 2.38-2.30 (m, 4H), 1.56-1.45 (m, 4H), 1.43-1.33 (m, 2H). MS (MH+): 568.6 | | | |
| I-11 | 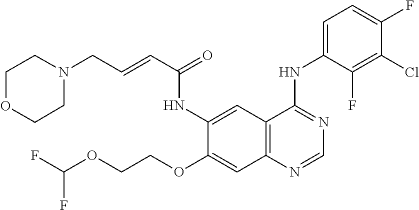 [SMILES: O=C(/C=C/CN1CCOCC1)Nc1cc2c(Nc3ccc(F)c(Cl)c3F)ncnc2cc1OCCOC(F)F] | (E)-N-[4-(3-chloro-2,4- difluoro-anilino)-7-[2- (difluoromethoxy)ethoxy] quinazolin-6-yl]-4- morpholino-but-2- enamide | White solid | 67 |
| | ¹H NMR (d6-DMSO, 300 MHz) δ 9.86 (s, 1H), 9.43 (s, 1H), 8.92 (s, 1H), 8.39 (s, 1H), | | | |
| | 7.56-7.44 (m, 1H), 7.40-7.29 (m, 1H), 7.32 (s, 1H), 6.85-6.71 (m, 1H), 6.80 (t, J = 75 | | | |
| | Hz, 1H), 6.53 (d, J = 18 Hz, 1H), 4.48-4.40 (m, 2H), 4.35-4.26 (m, 2H), 3.63-3.54 (m, | | | |
| | 4H), 3.14 (d, J = 6 Hz, 1H), 2.43-2.34 (m, 4H). MS (MH+): 570.5 | | | |
| I-12 | 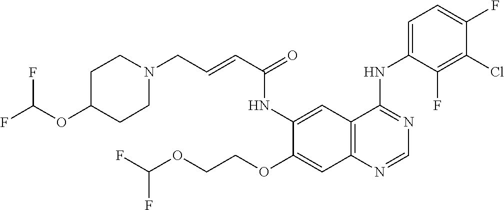 [SMILES: O=C(/C=C/CN1CCC(OC(F)F)CC1)Nc1cc2c(Nc3ccc(F)c(Cl)c3F)ncnc2cc1OCCOC(F)F] | (E)-N-[4-(3-chloro-2,4- difluoro-anilino)-7-[2- (difluoromethoxy)ethoxy] quinazolin-6-yl]-4-[4- (difluoromethoxy)-1- piperidyl]but-2-enamide | White solid | 27 |
| | ¹H NMR (d6-DMSO, 300 MHz) δ 9.86 (s, 1H), 9.42 (s, 1H), 8.91 (s, 1H), 8.39 (s, 1H), | | | |
| | 7.56-7.42 (m, 1H), 7.42-7.28 (m, 1H), 7.32 (m, 1H), 7.32 (s, 1H), 6.86-6.71 (m, 1H), | | | |
| | 6.80 (t, J = 76 Hz, 1H), 6.70 (t, J = 76 Hz, 1H), 6.50 (d, J = 15 Hz, 1H), 4.49-4.38 (m, | | | |
| | 2H), 4.34-4.26 (m, 2H), 4.16-4.04 (m, 1H), 3.14 (d, J = 6 Hz, 1H), 2.74-2.60 (m, 2H), | | | |
| | 2.29-2.11 (m, 2H), 1.94-1.79 (m, 2H), 1.70-1.54 (m, 2H). MS (MH+): 634.6 | | | |
| I-13 | 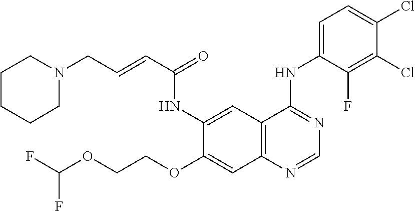 [SMILES: O=C(/C=C/CN1CCCCC1)Nc1cc2c(Nc3ccc(Cl)c(Cl)c3F)ncnc2cc1OCCOC(F)F] | (E)-N-[4-(3,4-dichloro-2- fluoro-anilino)-7-[2- (difluoromethoxy)ethoxy] quinazolin-6-yl]-4-(1- piperidyl)but-2-enamide | White solid | 50 |
| | ¹H NMR (d6-DMSO, 400 MHz) δ 9.92 (s, 1H), 9.41 (s, 1H), 8.93 (s, 1H), 8.41 (s, 1H), | | | |
| | 7.57-7.50 (m, 2H), 7.33 (s, 1H), 6.85-6.75 (m, 1H), 6.80 (t, J = 76 Hz, 1H), 6.50 (d, J = | | | |
| | 16 Hz, 1H), 4.48-4.40 (m, 2H), 4.34-4.27 (m, 2H), 3.09 (d, J = 8 Hz, 2H), 2.39-2.28 (m, | | | |
| | 4H), 1.55-1.46 (m, 4H), 1.42-1.33 (m. 2H). MS (MH+): 584.5 | | | |
| I-14 | 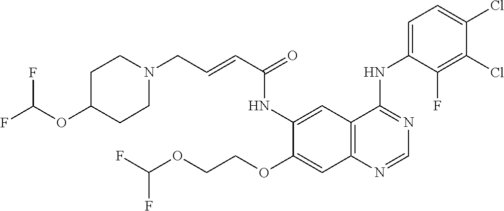 [SMILES: O=C(/C=C/CN1CCC(OC(F)F)CC1)Nc1cc2c(Nc3ccc(Cl)c(Cl)c3F)ncnc2cc1OCCOC(F)F] | (E)-N-[4-(3,4-dichloro-2- fluoro-anilino)-7-[2- (difluoromethoxy)ethoxy] quinazolin-6-yl]-4-[4- (difluoromethoxy)-l- piperidyl]but-2-enamide | White solid | 26 |
| | ¹H NMR (d6-DMSO, 400 MHz) δ 9.92 (s, 1H), 9.43 (s, 1H), 8.93 (s, 1H), 8.42 (s, 1H), | | | |
| | 7.57-7.50 (m, 2H), 7.33 (s, 1H), 6.83-6.74 (m, 1H), 6.80 (t, J = 76 Hz, 1H), 6.70 (t, J = | | | |
| | 76 Hz, 1H), 6.51 (d, J = 16 Hz, 1H), 4.47-4.41 (m, 2H), 4.34-4.28 (m, 2H), 4.16-4.07 | | | |
| | (m, 1H), 3.14 (d, J = 8 Hz, 1H), 2.71-2.61 (m, 2H), 2.25-2.14 (m, 2H), 1.91-1.81 (m, | | | |
| | 2H), 1.69-1.57 (m, 2H). MS (MH+): 650.6 | | | |
| I-15 | 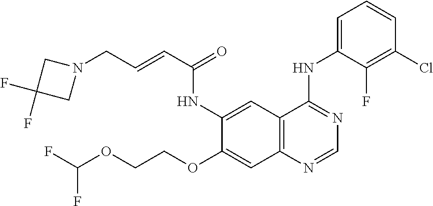 [SMILES: O=C(/C=C/CN1CC(F)(F)C1)Nc1cc2c(Nc3cccc(Cl)c3F)ncnc2cc1OCCOC(F)F] | (E)-N-[4-(3-chloro-2- fluoro-anilino)-7-[2- (difluoromethoxy)ethoxy] quinazolin-6-yl]-4-(3,3- difluoroazetidin-1-yl)but- 2-enamide | White solid | 18 |
| | ¹H NMR (d6-DMSO, 300 MHz) δ 9.88 (s, 1H), 9.63 (s, 1H), 8.91 (s, 1H), 8.40 (s, 1H), | | | |
| | 7.51-7.41 (m, 2H), 7.32 (s, 1H), 7.30-7.20 (m, 1H), 6.99-6.85 (m, 1H), 6.79 (t, J = 76 | | | |
| | Hz, 1H), 6.62 (d, J = 15 Hz, 1H), 4.49-4.38 (m, 2H), 4.36-4.26 (m, 2H), 3.65 (t, J = 12 | | | |
| | Hz, 4H), 3.40 (d, J = 3 Hz, 2H). MS (MH+): 558.7 | | | |
| I-16 | 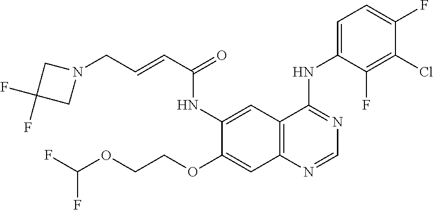 [SMILES: O=C(/C=C/CN1CC(F)(F)C1)Nc1cc2c(Nc3ccc(F)c(Cl)c3F)ncnc2cc1OCCOC(F)F] | (E)-N-[4-(3-chloro-2,4- difluoro-anilino)-7-[2- (difluoromethoxy)- ethoxy]quinazolin-6-yl]- 4-(3,3-difluoro-azetidin- 1-yl)but-2-enamide | White solid | 22 |
| | ¹H NMR (d6-DMSO, 300 MHz) δ 9.86 (s, 1H), 9.45 (s, 1H), 8.91 (s, 1H), 8.39 (s, 1H), | | | |
| | 7.55-7.44 (m, 1H), 7.40-7.31 (m, 1H), 7.32 (s, 1H), 6.80 (t, J = 76 Hz, 1H), 6.80-6.70 | | | |
| | (m, 1H), 6.51 (d, J = 18 Hz, 1H), 4.48-4.42 (m, 2H), 4.34-4.27 (m, 2H), 3.65 (t, J = 14 | | | |
| | Hz, 4H), 3.41 (d, J = 6 Hz, 2H). MS (MH+): 576.7 | | | |
| I-17 | 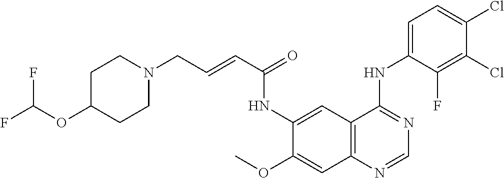 [SMILES: COc1cc2ncnc(Nc3ccc(Cl)c(Cl)c3F)c2cc1NC(=O)/C=C/CN1CCC(OC(F)F)CC1] | (E)-N-[4-(3,4-dichloro-2- fluoro-anilino)-7- methoxy-quinazolin-6- yl]-4-[4- (difluoromethoxy)-1- piperidyl]but-2-enamide | White solid | 14 |
| | ¹H NMR (d6-DMSO, 300 MHz) δ 9.90 (s, 1H), 9.66 (s, 1H), 8.94 (s, 1H), 8.41 (s, 1H), | | | |
| | 7.58-7.49 (m, 2H), 7.28 (s, 1H), 6.86-6.66 (m, 1H), 6.71 (t, J = 76 Hz, 1H), 6.60 (d, J = | | | |
| | 15 Hz, 1H), 4.17-4.04 (m, 1H), 4.01 (s, 3H), 3.16-3.09 (m, 2H), 2.74-2.59 (m, 2H), | | | |
| | 2.28-2.07 (m, 2H), 1.93-1.79 (m, 2H), 1.71-1.52 (m, 2H). MS (MH+): 570.5 | | | |
| I-18 | 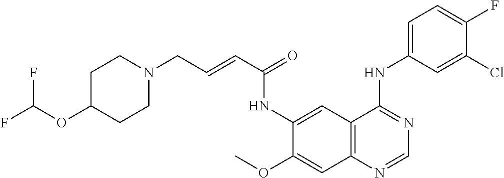 [SMILES: COc1cc2ncnc(Nc3ccc(F)c(Cl)c3)c2cc1NC(=O)/C=C/CN1CCC(OC(F)F)CC1] | (E)-N-[4-(3-chloro-4- fluoro-anilino)-7- methoxy-quinazolin-6- yl]-4-[4- (difluoromethoxy)-1- piperidyl]but-2-enamide | White solid | 40 |
| | ¹H NMR (d6-DMSO, 300 MHz) δ 9.78 (s, 1H), 9.67 (s, 1H), 8.91 (s, 1H), 8.52 (s, 1H), | | | |
| | 8.12 (dd, J = 6, 3 Hz, 1H), 7.82-7.75 (m, 1H), 7.40 (t, J = 10 Hz, 1H), 7.27 (s, 1H) | | | |
| | 6.85-6.72 (m, 1H), 6.71 (t, J = 76 Hz, 1H), 6.56 (d, J = 15 Hz, 1H), 4.18-4.05 (m, 1H), | | | |
| | 3.13 (d, J = 6 Hz, 1H), 2.73-2.61 (m, 2H), 2.27-2.12 (m, 2H), 1.94-1.80 (m, 2H), 1.71- | | | |
| | 1.55 (m,2H). MS (MH+): 536.6 | | | |
| I-19 | 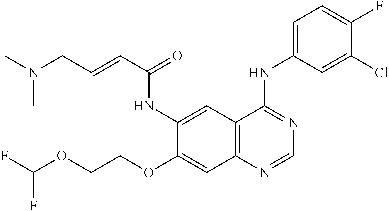 [SMILES: CN(C)C/C=C/C(=O)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy] quinazolin-6-yl]-4- (dimethylamino)but-2- enamide | White solid | 20 |
| | ¹H NMR (d6-DMSO, 300 MHz) δ 9.81 (s, 1H), 9.45 (s, 1H), 8.92 (s, 1H), 8.52 (s, 1H), | | | |
| | 8.12 (dd, J = 6, 3 Hz, 1H), 7.82-7.75 (m, 1H), 7.41 (t, J = 9 Hz, 1H), 7.32 (s, 1H), 6.84- | | | |
| | 6.74 (m, 1H), 6.79 (t, J = 75 Hz, 1H), 6.52 (d, J = 15 Hz, 1H), 4.46-4.40 (m, 2H), 4.33- | | | |
| | 4.27 (m, 2H), 3.08 (d, J = 3 Hz, 2H), 2.17 (s, 6H). MS (MH+): 510.6 | | | |
| I-20 | 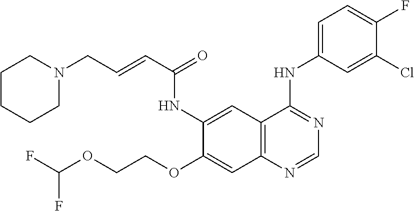 [SMILES: O=C(/C=C/CN1CCCCC1)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy] quinazolin-6-yl]-4-(1- piperidyl)but-2-enamide | White solid | 76 |
| | ¹H NMR (d6-DMSO, 300 MHz) δ 9.80 (s, 1H), 9.44 (s, 1H), 8.90 (s, 1H), 8.52 (s, 1H), | | | |
| | 8.12 (dd, J = 6, 3 Hz, 1H), 7.82-7.74 (m, 1H), 7.41 (t, J = 9 Hz, 1H), 7.32 (s, 1H), 6.85- | | | |
| | 6.73 (m, 1H), 6.79 (t, J = 75 Hz, 1H), 6.48 (d, J = 15 Hz, 1H), 4.47-4.39 (m, 2H), 4.33- | | | |
| | 4.26 (m, 2H), 3.10 (d, J = 3 Hz, 2H), 2.39-2.28 (m, 4H), 1.57-1.44 (m, 4H), 1.44-1.32 | | | |
| | (m, 2H). MS (MH+): 550.7 | | | |
| I-21 | 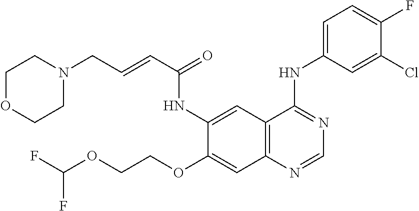 [SMILES: O=C(/C=C/CN1CCOCC1)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy] quinazolin-6-yl]-4- morpholino-but-2- enamide | White solid | 92 |
| | ¹H NMR (d6-DMSO, 300 MHz) δ 9.80 (s, 1H), 9.46 (s, 1H), 8.90 (s, 1H), 8.52 (s, 1H), | | | |
| | 8.12 (dd, J = 6, 3 Hz, 1H), 7.82-7.74 (m, 1H), 7.41 (t, J = 9 Hz, 1H), 7.32 (s, 1H), 6.84- | | | |
| | 6.73 (m, 1H), 6.79 (t, J = 75 Hz, 1H), 6.52 (d, J = 15 Hz, 1H), 4.46-4.40 (m, 2H), 4.33- | | | |
| | 4.27 (m, 2H), 3.63-3.54 (m, 4H), 3.13 (d, J = 6 Hz, 2H), 2.44-2.34 (m, 4H). | | | |
| | MS (MH+): 552.7 | | | |
| I-22 | 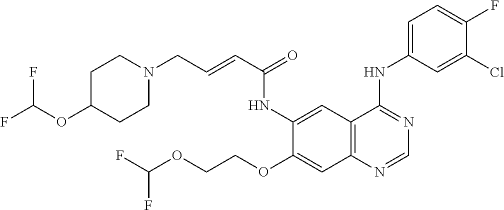 [SMILES: O=C(/C=C/CN1CCC(OC(F)F)CC1)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy] quinazolin-6-yl]-4-[4- (difluoromethoxy)-l- piperidyl]but-2-enamide | White solid | 88 |
| | ¹H NMR (d6-DMSO, 300 MHz) δ 9.80 (s, 1H), 9.45 (s, 1H), 8.90 (s, 1H), 8.52 (s, 1H), | | | |
| | 8.12 (dd, J = 6, 3 Hz, 1H), 7.82-7.75 (m, 1H), 7.41 (t, J = 9 Hz, 1H), 7.32 (s, 1H), 6.84- | | | |
| | 6.74 (m, 1H), 6.79 (t, J = 75 Hz, 1H), 6.71 (t, J = 75 Hz, 1H), 6.50 (d, J = 15 Hz, 1H), | | | |
| | 4.47-4.40 (m, 2H), 4.33-4.26 (m, 2H), 4.18-4.06 (m, 1H), 3.14 (d, J = 6 Hz, 2H), 2.74- | | | |
| | 2.61 (m, 2H), 2.28-2.14 (m, 2H), 1.93-1.81 (m, 2H), 1.70-1.55 (m, 2H). | | | |
| | MS (MH+): 616.0 | | | |
| I-23 | 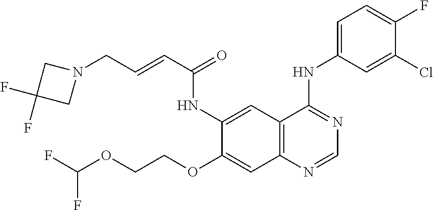 [SMILES: O=C(/C=C/CN1CC(F)(F)C1)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy] quinazolin-6-yl]-4-(3,3- difluoroazetidin-1-yl)but- 2-enamide | White solid | 20 |
| | ¹H NMR (d6-DMSO, 300 MHz) δ 9.80 (s, 1H), 9.48 (s, 1H), 8.90 (s, 1H), 8.52 (s, 1H), | | | |
| | 8.12 (dd, J = 6, 3 Hz, 1H), 7.82-7.75 (m, 1H), 7.40 (t, J = 9 Hz, 1H), 7.32 (s, 1H), 6.81- | | | |
| | 6.72 (m, 1H), 6.80 (t, J = 75 Hz, 1H), 6.50 (d, J = 15 Hz, 1H), 4.47-4.40 (m, 2H), 4.33- | | | |
| | 4.27 (m, 2H), 3.65 (t, J = 12 Hz, 4H), 3.40 (d, J = 3 Hz, 2H). MS (MH+): 558.5 | | | |
| I-24 | 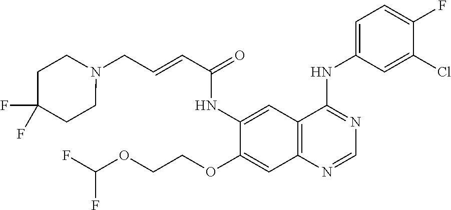 [SMILES: O=C(/C=C/CN1CCC(F)(F)CC1)Nc1cc2c(Nc3ccc(F)c(Cl)c3)ncnc2cc1OCCOC(F)F] | (E)-N-[4-(3-chloro-4- fluoro-anilino)-7-[2- (difluoromethoxy)ethoxy] quinazolin-6-yl]-4-(4,4- difluoro-1-piperidyl)but- 2-enamide | White solid | 35 |

¹H NMR (d6-DMSO, 300 MHz) δ 9.80 (s, 1H), 9.46 (s, 1H), 8.90 (s, 1H), 8.52 (s, 1H),
8.12 (dd, J = 6, 3 Hz, 1H), 7.83-7.75 (m, 1H), 7.41 (t, J = 9 Hz, 1H), 7.32 (s, 1H), 6.85-
6.75 (m, 1H), 6.79 (t, J = 75 Hz, 1H), 6.52 (d, J = 15 Hz, 1H), 4.46-4.41 (m, 2H), 4.32-
4.27 (m, 2H), 3.23 (d, J = 6 Hz, 2H), 2.56-2.49 (m, 4H), 2.06-1.89 (m, 4H). MS (MH+): 586.6

### Example 11: 7-[2-(difluoromethoxy)ethoxy]-N-[2-fluoro-3-(2-trimethylsilylethynyl)phenyl]-6-nitro-quinazolin-4-amine (U)

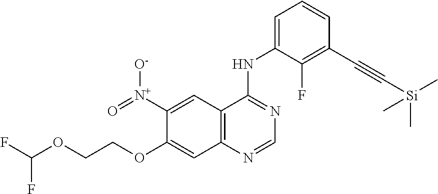 [SMILES: C[Si](C)(C)C#Cc1cccc(Nc2ncnc3cc(OCCOC(F)F)c([N+](=O)[O-])cc23)c1F]

To a stirred solution of N-(3-bromo-2-fluoro-phenyl)-7-[2-(difluoromethoxy)ethoxy]-6-nitro-quinazolin-4-amine (Example 5, compounds E-7, 4.93 g, 12.53 mmol) in dioxane (50 mL) and trimethylamine (20 mL) under argon was added copper(I) iodide (110 mg), TMS-acetylene (8.9 mL, 62.66 mmol) and Pd(PPh₃)₂Cl₂. The mixture was stirred under an inert atmosphere at 60° C. for 6 h. The mixture was diluted with ethyl acetate and washed with brine (3×). The organic phase was dried, filtered and concentrated in vacuo then chromatographed in 8% ethyl acetate in DCM to give the desired product (3.61 g, 71%). Mass: 490.12, used directly in the subsequent reaction.

### Example 12: 7-[2-(difluoromethoxy)ethoxy]-N4-[2-fluoro-3-(2-trimethylsilylethynyl)phenyl]quinazoline-4,6-diamine (V)

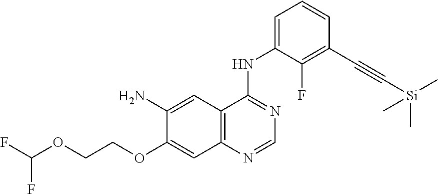 [SMILES: C[Si](C)(C)C#Cc1cccc(Nc2ncnc3cc(OCCOC(F)F)c(N)cc23)c1F]

To a stirred solution of 7-[2-(difluoromethoxy)ethoxy]-N-[2-fluoro-3-(2-trimethylsilylethynyl)phenyl]-6-nitro-quinazolin-4-amine (Example 11, 1.1 g, 2.29 mmol) in methanol (64 mL) and THF (21 mL) cooled to 0° C. was added saturated aqueous ammonium chloride (8.5 mL), followed by zinc dust (1.49 g, 22.9 mmol). The mixture was stirred for 5 min at 0° C. then at room temperature for 30 min. The mixture was then diluted with water and extracted with ethyl acetate (2×). The organic phases were combined and washed with brine, dried over anhydrous magnesium sulfate, filtered and concentrated to give the crude product (1.0 g, 95%) used directly in the subsequent reaction. Mass: 460.15.

### Example 13: (E)-N-[7-[2-(difluoromethoxy)ethoxy]-4-[2-fluoro-3-(2-trimethylsilylethynyl)anilino]quinazolin-6-yl]-4-(dimethylamino)but-2-enamide

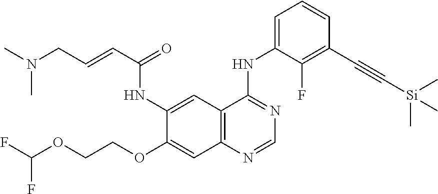 [SMILES: CN(C)C/C=C/C(=O)Nc1cc2c(Nc3cccc(C#C[Si](C)(C)C)c3F)ncnc2cc1OCCOC(F)F]

(E)-4-bromo-N-[7-[2-(difluoromethoxy)ethoxy]-4-[2-fluoro-3-(2-trimethylsilylethynyl)anilino]quinazolin-6-yl]but-2-enamide (Example 8, Compound J-9, 90 mg, 0.148 mmol), dimethylamine hydrochloride (36 mg, 0.443 mmol) potassium carbonate (61 mg, 0.443 mmol) and potassium iodide (5 mg) were stirred in DMF (3 mL) at room temperature for 3 h. The mixture was diluted with ethyl acetate and washed with brine. The organic phase was dried, filtered and concentrated in vacuo then chromatographed in 3-4% MeoH in DCM to give the desired product as an off-white solid (56 mg, 67%).

### Example 14: (E)-N-[7-[2-(difluoromethoxy)ethoxy]-4-(3-ethynyl-2-fluoro-anilino)quinazolin-6-yl]-4-(dimethylamino)but-2-enamide (I-25)

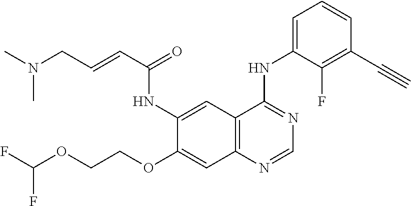 [SMILES: C#Cc1cccc(Nc2ncnc3cc(OCCOC(F)F)c(NC(=O)/C=C/CN(C)C)cc23)c1F]

To a stirred solution of (E)-N-[7-[2-(difluoromethoxy)ethoxy]-4-[2-fluoro-3-(2-trimethylsilylethynyl)anilino]quinazolin-6-yl]-4-(dimethylamino)but-2-enamide (Example 12, 56 mg, 0.098 mmol) in THF (2 mL) cooled to 0° C. was added TBAF (1 M solution, 100 μL) and the mixture was stirred for 30 min. The mixture was diluted with ethyl acetate and washed with water and brine. The organic phase was dried, filtered and concentrated in vacuo then chromatographed, eluting with 4-5% MeOH in DCM. The product containing fractions were concentrated in vacuo and triturated with DCM and hexanes to give the desired product as an off-white solid (25 mg, 56%).

### Example 15: Preparation of (E)-N-[4-(3-chloro-2-fluoro-anilino)-7-[2-(difluoromethoxy)ethoxy]quinazolin-6-yl]-4-(dimethylamino)but-2-enamide dimaleic acid salt (I-5-MA)

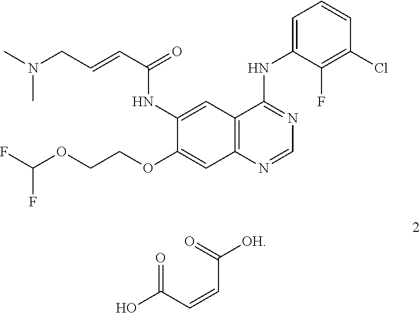 [SMILES: CN(C)C/C=C/C(=O)Nc1cc2c(Nc3cccc(Cl)c3F)ncnc2cc1OCCOC(F)F ; O=C(O)/C=C\C(=O)O]

To a stirred solution of (E)-N-[4-(3-chloro-2-fluoro-anilino)-7-[2-(difluoromethoxy)ethoxy]quinazolin-6-yl]-4-(dimethylamino)but-2-enamide(460 mg, 0.902 mmol) in THF (8 mL) under ice-water bath, maleic acid (209 mg, 1.804 mmol) in THF (2 mL) was added. After being stirred for 20 minutes, the reaction mixture was concentrated by Rotavapor and resulting product was triturated with acetone and dichloromethane to give the desired product (600 mg, 90%) as an off-white solid. 1H NMR (d6-DMSO, 400 MHz) δ 10.0 (br, 1H), 975 (s, 1H), 8.92 (s, 1H), 8.44 (s, 1H), 7.52-7.42 (m, 2H), 7.35 (s, 1H), 7.30-7.22 (m, 1H), 6.88-6.67 (m, 2H), 6.79 (t, J=78 Hz, 1H), 6.12 (s, 4H), 4.49-4.44 (m, 2H), 4.33-4.23 (m, 2H), 3.96 (d, J=8 Hz, 2H), 2.79 (s, 6H).

In a similar manner, using the above procedure, the following (E)-N-aryl)-7-[2-(difluoromethoxy)ethoxy] or methoxy-quinazolin-6-yl]-4-(dimethylamino)-but-2-enamide were synthesized as maleate salts.

TABLE I

| Exam- | | | | |
|---|---|---|---|---|
| ple | | | Appear- | Yield |
| # | Structure | Nomenclature | rance | (%) |
| I-5-MA | 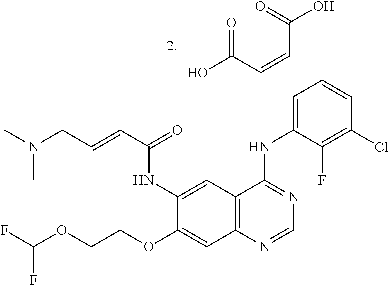 | (E)-N-[4-(3- chloro-2-fluoro- anilino)-7-[2- (difluoromethoxy) ethoxy]quinazolin- 6-yl]-4- (dimethylamino) but-2-enamide di-maleic acid salt | White solid | 90 |
| | 1H NMR (d6-DMSO, 400 MHz) δ 10.0 (br, 1H), 975 (s, 1H), 8.92 (s, 1H), 8.44 (s, 1H), | | | |
| | 7.52-7.42 (m, 2H), 7.35 (s, 1H), 7.30-7.22 (m, 1H), 6.88-6.67 (m, 2H), 6.79 (t, J = 78 Hz, | | | |
| | 1H), 6.12 (s, 4H), 4.49-4.44 (m, 2H), 4.33-4.23 (m, 2H), 3.96 (d, J = 8 Hz, 2H), 2.79 (s, 6H). | | | |
| I-9-MA | 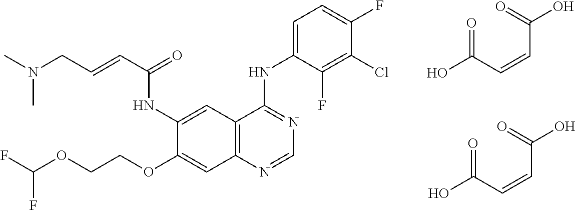 [SMILES: CN(C)C/C=C/C(=O)Nc1cc2c(Nc3ccc(F)c(Cl)c3F)ncnc2cc1OCCOC(F)F ; O=C(O)/C=C\C(=O)O ; O=C(O)/C=C\C(=O)O] | (E)-N-[4-(3- chloro-2,4- difluoro- anilino)-7-[2- (difluoromethoxy) ethoxy]quinazolin- 6-yl]-4- (dimethylamino) but-2-enamide di-maleic acid salt | White solid | 84 |
| | 1H NMR (d6-DMSO, 400 MHz) δ 9.95 (br, 1H), 974 (s, 1H), 8.90 (s, 1H), | | | |
| | 8.43 (s, 1H), 7.52-7.46 (m, 1H), 7.42- 7.30 (m, 2H), 6.85-6.66 (m, 2H), 6.78 (t, J = 78 Hz, | | | |
| | 1H), 6.12 (s, 4H), 4.50-4.43 (m, 2H), 4.403-4.25 (m, 2H), 3.96 (d, J=8 Hz, 2H), 2.79 (s, 6H). | | | |
| I-10-MA | 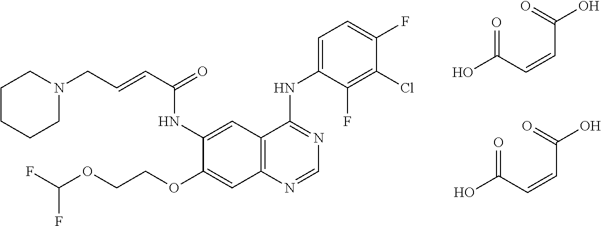 [SMILES: O=C(/C=C/CN1CCCCC1)Nc1cc2c(Nc3ccc(F)c(Cl)c3F)ncnc2cc1OCCOC(F)F ; O=C(O)/C=C\C(=O)O ; O=C(O)/C=C\C(=O)O] | (E)-N-[4-(3-chloro- 2,4-difluoro- anilino)-7-[2- (difluoromethoxy) ethoxy]quinazolin- 6-yl]-4-(1-piperidyl) but-2-enamide di-maleic acid salt | White solid | 93 |
| | 1H NMR (d6-DMSO, 400 MHz) δ 9.94 (br, 1H), 973 (s, 1H), 8.89 (s, 1H), 8.43 (s, 1H), | | | |
| | 7.51-7.45 (m, 1H), 7.43-7.32 (m, 2H), 6.84-6.68 (m, 2H), 6.78 (t, J = 78 Hz, 1H), 6.12 (s, | | | |
| | 4H), 4.50-4.44 (m, 2H), 4.35-4.26 (m, 2H), 3.95 (d, J = 8 Hz, 2H), 3.10-2.80 (m, 4H), 1.90-1.50 (m, 6H). | | | |
| I-11-MA | 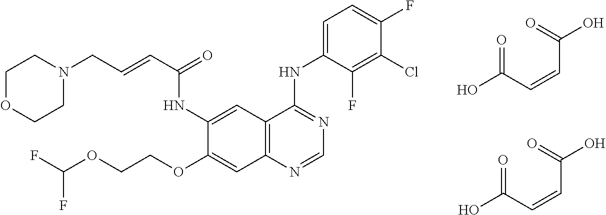 [SMILES: O=C(/C=C/CN1CCOCC1)Nc1cc2c(Nc3ccc(F)c(Cl)c3F)ncnc2cc1OCCOC(F)F ; O=C(O)/C=C\C(=O)O ; O=C(O)/C=C\C(=O)O] | (E)-N-[4-(3-chloro- 2,4-difluoro- anilino)-7-[2- difluoromethoxy) ethoxy]quinazolin- 6-yl]-4- morpholino-but- 2-enamide di-maleic acid | White solid | 90 |

1H NMR (d6-DMS0, 300 MHz) δ 10.12 (br, 1H), 9.68 (s, 1H), 8.92 (s, 1H), 8.43 (s, 1H),
7.58-7.45 (m, 1H), 7.42- 7.36 (m, 1H), 7.34 (s, 1H), 6.87-6.64 (m, 2H), 6.79 (t, J = 75 Hz,
1H), 6.15 (s, 4H), 4.48-4.42 (m, 2H), 4.35-4.26 (m, 2H), 3.86-3.70 (m, 6H), 3.10-2.97 (m, 4H).

### Biological Testing

### Example 16: Binding to EPHA

For most kinase assays, kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). All reactions were performed in polystyrene 96-well plates in a final volume of 0.135 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, and 0.5 μM non-biotinylated affinity ligand) and incubated at room temperature with shaking for 30 minutes. The kinase concentration in the eluates was measured by qPCR.

Table 1 shows the results for compounds I-9-MA and I-10-MA. Both compounds are selective for EPHA6 with a % control @ 300 nM of 3.5% and 3.9%, respectively.

TABLE 1

Example #

| | |
|---|---|
| I-9-MA | I-10-MA |

| | |
|---|---|
| | % Control @ |
| Gene Symbol | 300 nM |

| | | |
|---|---|---|
| EPHA1 | 34 | 24 |
| EPHA2 | 92 | 80 |
| EPHA3 | 46 | 45 |
| EPHA4 | 51 | 50 |
| EPHA5 | 51 | 49 |
| EPHA6 | 3.5 | 3.9 |
| EPHA7 | 56 | 40 |
| EPHA8 | 40 | 33 |

### Example 17: Determination of Kinase Activity: IC₅₀

### Selectivity Against WT EGFR, Mutant EGFR and Ephrin Receptor Tyrosine Kinases

### Kinase Assays.

For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 40× stocks in 100% DMSO and directly diluted into the assay. All reactions were performed in polypropylene 384-well plates in a final volume of 0.04 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

### Results & Discussion

Compounds I-9-MA and I-10-MA were assessed against a panel of 11 WT EGFR, mutant EGFR and ephrin receptor tyrosine kinases. Ultrasensitive quantitative PCR (qPCR) was used to measure levels of immobilized kinases after treatment with compounds I-9-MA and I-10-MA at 300 nM. Both compounds did not show selectivity against WT EGFR and mutant EGFR kinases (see Table 2).

TABLE 2

| | |
|---|---|
| I-9-MA | I-10-MA |

| | | |
|---|---|---|
| | | % Control @ |
| DiscoveRx Gene Symbol | Symbol | 300 nM |

| | | | |
|---|---|---|---|
| EGFR | EGFR | 0.7 | 2.7 |
| EGFR (E746-A750del) | EGFR | 3.6 | 2.7 |
| EGFR (G719C) | EGFR | 1.1 | 0 |
| EGFR (G719S) | EGFR | 0.15 | 0 |
| EGFR (L747-E749del, | EGFR | 0.45 | 0.9 |
| A750P) | | | |
| EGFR (L747-S752del, | EGFR | 2.8 | 2.5 |
| P753S) | | | |
| EGFR (L747- | EGFR | 8.6 | 0 |
| T751del, Sins) | | | |
| EGFR (L858R) | EGFR | 0 | 0.4 |
| EGFR (L858R, T790M) | EGFR | 3.5 | 2.4 |
| EGFR (L861Q) | EGFR | 0.1 | 0.75 |
| EGFR (S752-I759del) | EGFR | 0.5 | 0 |
| EGFR (T790M) | EGFR | 0.1 | 0.75 |
| ERBB2 | ERBB2 | 0 | 0.05 |
| ERBB4 | ERBB4 | 1.8 | 3.2 |
| BLK (B-lymphoid TK) | BLK | 0.5 | 0.3 |
| EPHA6 | EPHA6 | 3.5 | 3.9 |
| LCK (Lymphocyte TK) | LCK | 1.3 | 1.3 |
| SRC | SRC | 3.5 | 4.8 |

### Example 18: Evaluation of P-gp Efflux

P-glycoprotein (Pgp) is a member of the ABC-transporter family that transports substances across cellular membranes acting as an energy-dependent efflux pump extruding drugs out of the cells. Increased expression of Pgp in cancer cells is one of the major mechanisms of cancer resistances and chemotherapy and thus Pgp plays a key role on the pharmacokinetics of drug absorption and distribution.

### Protocol

Human, epithelial Caco-2 cells (CRL-2102 (C2BBe1)) were seeded at a density of 40,000 cells/well, on high-density PET membrane inserts, (1.0 μm pore size, 0.31 cm² surface area) and utilized on day 21 or 22 days (post-seeding). At this stage of growth, cell monolayers were fully polarized and differentiated.

The permeability assay buffer was Hanks Balanced Salt Solution containing 10 mM HEPES and 15 mM glucose at a pH of 7.4. The dosing buffer contained 5 μM metoprolol (positive control), 5 μM atenolol (negative control) and 100 μM lucifer yellow. The buffer in the receiver chamber also contained 1% bovine serum albumin (BSA). The dosing solution concentration was 5 μM in the assay buffer. Digoxin (20 μM) was used as Pgp substrate control.

For suspected Pgp substrate, the assays were performed with and without a known Pgp inhibitor (e.g. Verapamil or Ketoconazole). The known Pgp inhibitor was co-dosed at 50 μM with compound at 5 μM.

Cell monolayers were dosed on the apical side (A-to-B) or basolateral side (B-to-A) and incubated at 37° C. in a shaker (65 rpm). Samples were taken from the donor and receiver chambers at 120 minutes. Each determination was performed in duplicate.

Narrow-window mass extraction LC/MS analysis was performed for all samples from this study using a Waters Xevo quadrupole time-of-flight (QTof) mass spectrometer, to determine relative peak areas of parent compound. The percent of transported drug was calculated based on these peak areas, relative to the initial, dosing concentration.

### Results

Activity as a Pgp substrate is an undesirable trait for anti cancer compound. Compounds that are substrates for Pgp will be more readily transported out of a cancer cell and therefore show reduced activity. In the present assay it is desirable for a compound to show an efflux ratio of between about 1 and 5. Results for representative compounds of the application are shown in Table 3. As can be seen, compounds I-5-MA and I-7-MA show increased concentrations at target organs when compared to Afatinib and have a significantly lower efflux ratio. In fact the efflux ratio for both compounds is well within, and even below, the desirable range for anticancer drugs.

TABLE 3
Permeability results of I-5-MA, I-7-MA and afatinib compared control
drugs Propranolol, Prazosin and Digoxin in Caco-2 cell line

| | Papp (A-B) | Papp (B-A) | |
|---|---|---|---|
| Example# | (10−6, cm/s) | (10−6, cm/s) | Efflux Ratio |
| Propranolol | 21.06 | 15.76 | 0.75 |
| Prazosin | 21.74 | 34.67 | 1.59 |
| Digoxin | 0.75 | 22.74 | 30.39 |
| Afatinib | 0.81 | 16.01 | 19.87 |
| I-5-MA | 13.46 | 6.52 | 0.48 |
| I-7-MA | 2.43 | 9.24 | 3.80 |

### Example 19: National Cancer Institute (NCI) Screening Panel

Screening of Compounds I-9-MA and I-10-MA within the NCI Panel

Compounds I-9-MA and I-10-MA were screened using the National Cancer Institute (NCI) screening panel, which consists of a panel of 60 different human tumor cell lines, representing leukemia [CCRF-CEM, HL-60 (TB), K-562, MOLT-4, SR], melanoma [LOX IMVI, MALME-3M, M14, SMDA-MB-435, SK-MEL-2, SK-MEL-28, SK-MEL-5, UACC-257 and UACC-62] and cancers of the lung [A549/ATCC, EKVX, HOP-62, HOP-93, NCI-H226, NCI-H23, NCI-H322M, NCI-H460], colon [COLO 205, HCT-116, HCT-15, HT29, KM12, SW-620], brain [SF-268, SF-295, SF-539, SNB-19, SNB-75, U251], ovary [IGROV1, OVCAR-3, OVCAR-4, OVCAR-5, OVCAR-8, NCI/ADR-RES, SK-OV-3], breast [MCF7, MDA-MB-231, BT-549, T-47D, MDA-MB-468], prostate [PC-3, DU-145], and renal [786-0, A498, ACHN, CAKI-1, RXF-393, SN12C, TK-10, UO-31] cancers.

After 24 h, two plates of each cell line are fixed in situ with TCA, to represent a measurement of the cell population for each cell line at the time of drug addition (T_{z}). Experimental drugs are solubilised in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 h at 37° C., 5% CO₂, 95% air, and 100% relative humidity. For adherent cells, the assay is terminated by the addition of cold TCA (trichloroacetic acid). Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilised with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (T_{z}), control growth, (C), and test growth in the presence of drug at the five concentration levels (Tᵢ)], the percentage growth is calculated at each of the drug concentration levels. Percentage growth inhibition is calculated as: [(Tᵢ−T_{z})/(C−T_{z})]×100 for concentrations in which Tᵢ>/=T_{z} and [(Tᵢ−T_{z})/T_{z}]×100 for concentrations in which Tᵢ<T_{z}.

Three dose response parameters are calculated for each experimental agent. Growth inhibition of 50% (GI₅₀) is calculated from [(Tᵢ−T_{z})/(C−T_{z})]×100=50, which is the drug concentration resulting in a 50% reduction in the net protein increase (as measured by SRB staining) in control cells during the drug incubation. The drug concentration resulting in total growth inhibition (TGI) is calculated from Tᵢ=T_{z}. The LC₅₀ (concentration of drug resulting in a 50% reduction in the measured protein at the end of the drug treatment as compared to that at the beginning) indicating a net loss of cells following treatment is calculated from [(Tᵢ−T_{z})/T_{z}]×100=−50. Values are calculated for each of these three parameters if the level of activity is reached. However, if the effect is not reached or is exceeded, the value for that parameter is expressed as greater or less than the maximum or minimum concentration tested.

The results obtained from this study shows compound I-9-MA and I-10-MA are effective against the cell lines of the 60 human tumor cell lines panel. Inhibition of human cancer cell lines in vitro by the exemplified compounds is shown in Table 4.

TABLE 4
NCI 60-panel screen results

| | Example# | | | |
|---|---|---|---|---|
| | I-9-MA | | I-10-MA | |
| | GI50 | LC50 | GI50 | LC50 |
| Leukemia | | | | |
| CCRF-CEM | 2.47E−06 | >1.00E−4 | 2.27E−06 | >1.00E−4 |
| HL-60(TB) | 1.73E−06 | 3.22E−05 | 1.75E−06 | 3.08E−05 |
| K-562 | 4.59E−07 | 6.19E−05 | 1.03E−06 | >1.00E−4 |
| MOLT-4 | 8.49E−07 | 4.75E−05 | 1.12E−06 | 9.84E−05 |
| RPMI-8226 | 1.29E−06 | >1.00E−4 | 1.64E−06 | 5.89E−05 |
| SR | 4.39E−07 | 5.92E−05 | 8.16E−07 | >1.00E−4 |
| NSCLC | | | | |
| A549/ATCC | 7.70E−07 | 3.63E−05 | 1.00E−06 | 3.66E−05 |
| EKVX | <1.00E−8 | 1.96E−05 | 3.47E−08 | 2.39E−05 |
| HOP-62 | 1.35E−06 | 4.15E−05 | 1.62E−06 | 3.95E−05 |
| HOP-92 | 1.85E−07 | 3.12E−05 | 1.51E−07 | 2.11E−05 |
| NCI-H226 | 1.71E−06 | 9.70E−06 | 1.77E−06 | >1.00E−4 |
| NCI-H23 | 1.38E−06 | 1.54E−05 | 2.09E−06 | >1.00E−4 |
| NCI-H322M | <1.00E−8 | 2.82E−05 | <1.00E−8 | 3.03E−05 |
| NCI-H460 | 2.56E−06 | 6.20E−05 | 2.34E−06 | 5.44E−05 |
| NCI-H522 | <1.00E−8 | 9.17E−06 | 5.59E−08 | 7.80E−06 |
| Colon | | | | |
| COLO205 | 1.23E−06 | 3.17E−05 | 1.74E−06 | >1.00E−4 |
| HCC-2998 | 1.14E−06 | 5.67E−06 | 1.80E−06 | 7.85E−06 |
| HCT-116 | 4.83E−07 | 2.24E−05 | 1.54E−06 | 8.89E−05 |
| HCT-15 | 6.75E−07 | 9.46E−06 | 1.34E−06 | 2.30E−05 |
| HT29 | 3.83E−07 | 6.26E−05 | 1.14E−06 | 5.14E−05 |
| KM12 | 1.88E−06 | 2.92E−05 | 2.67E−06 | 3.43E−05 |
| SW-620 | 1.52E−06 | 5.27E−05 | 1.81E−06 | 3.47E−05 |
| CNS | | | | |
| SF-268 | 7.47E−07 | 2.73E−05 | 1.25E−06 | 4.17E−05 |
| SF-295 | 2.05E−06 | 3.30E−05 | 1.91E−06 | 1.60E−05 |
| SF-539 | 1.06E−06 | 9.52E−06 | 1.50E−06 | 1.43E−05 |
| SNB-19 | 1.60E−06 | 3.66E−05 | 2.16E−06 | 6.88E−05 |
| SNB-75 | 1.29E−07 | 2.36E−05 | 2.74E−07 | 3.10E−05 |
| U251 | 2.17E−06 | 2.96E−05 | 2.17E−06 | 4.07E−05 |
| Melanoma | | | | |
| LOXIMVI | 9.44E−07 | 4.91E−06 | 1.34E−06 | 5.49E−06 |
| MALME3-M | 1.42E−06 | 6.97E−06 | 1.45E−06 | 7.44E−06 |
| M14 | 1.44E−06 | 1.10E−05 | 1.82E−06 | >1.00E−4 |
| MDA-MB-435 | 1.17E−06 | 2.72E−05 | 1.80E−06 | 2.86E−05 |
| SK-MEL-28 | 2.29E−06 | 3.62E−05 | 2.49E−06 | 3.65E−05 |
| SK-MEL-5 | 1.65E−06 | 7.39E−06 | 1.63E−06 | 6.25E−06 |
| UACC-257 | 2.18E−06 | 3.40E−05 | 1.74E−06 | 2.41E−05 |
| UACC-62 | 1.49E−06 | 9.95E−06 | 1.79E−06 | 2.05E−05 |
| Ovarian | | | | |
| IGROV1 | <1.00E−8 | 3.25E−05 | 3.31E−08 | >1.00E−4 |
| OVCAR-3 | 8.11E−08 | 7.44E−06 | 3.90E−07 | 1.68E−05 |
| OVCAR-4 | 9.99E−07 | 3.24E−05 | 1.31E−06 | 5.99E−05 |
| OVCAR-5 | 1.41E−07 | 4.79E−05 | 7.76E−07 | 6.71E−05 |
| OVCAR-8 | 1.09E−06 | 3.27E−05 | 1.35E−06 | >1.00E−4 |
| NCI/ADR-RES | 8.39E−07 | >1.00E−4 | 1.00E−06 | >1.00E−4 |
| SK-OV-3 | <1.00E−8 | 2.77E−05 | 4.05E−08 | 3.60E−05 |
| Renal | | | | |
| 786-0 | 2.48E−07 | 2.66E−05 | 9.29E−07 | >1.00E−4 |
| A498 | 3.19E−07 | 6.00E−06 | 3.13E−07 | 5.92E−06 |
| ACHN | <1.00E−8 | 6.01E−06 | <1.00E−8 | 1.50E−05 |
| CAKI-1 | <1.00E−8 | 3.76E−06 | 2.29E−08 | 6.53E−06 |
| RXF393 | 3.51E−07 | 1.51E−05 | 9.59E−07 | 3.33E−05 |
| SN12C | 5.40E−08 | 2.81E−05 | 2.81E−07 | 4.12E−05 |
| TK-10 | <1.00E−8 | 2.18E−05 | <1.00E−8 | 3.51E−05 |
| UO-31 | <1.00E−8 | 1.46E−05 | 1.34E−08 | 3.91E−05 |
| Prostate | | | | |
| PC-3 | 2.55E−06 | 5.85E−05 | 2.38E−06 | 5.10E−05 |
| DU-145 | 1.40E−07 | 2.08E−05 | 2.66E−07 | 3.78E−05 |
| Breast | | | | |
| MCF7 | 1.40E−06 | 5.78E−05 | 1.84E−06 | 5.98E−05 |
| MDA-MB- | 3.91E−07 | 2.88E−05 | 1.15E−06 | 1.76E−05 |
| 231/ATCC | | | | |
| HS578T | 1.24E−06 | >1.00E−4 | 1.41E−06 | >1.00E−4 |
| BT-549 | 1.69E−06 | 3.22E−05 | 2.47E−06 | 3.62E−05 |
| T-47D | <1.00E−8 | 8.81E−05 | 8.14E−08 | >1.00E−4 |
| MDA-MB-468 | <1.00E−8 | 7.01E−07 | <1.00E−8 | 6.93E−06 |

### Example 20: Kinase HotSpot Profiling

### Reagents:

Base Reaction buffer; 20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO
Required cofactors are added individually to each kinase reaction

### Reaction Procedure:

1. The indicated substrate was prepared in fresh Base Reaction Buffer.

2. Any required cofactors were added to the substrate solution above.

3. Indicated kinase was added into the substrate solution and gently mixed.

4. Compounds in DMSO were added into the kinase reaction mixture by Acoustic technology (Echo550; nanoliter range) and incubated for 20 minutes at room temperature.

5. 33P-ATP (specific activity 10 μCi/μl) was added into the reaction mixture to initiate the reaction.

6. The kinase reaction was incubated for 2 hours at room temperature

7. Reactions were spotted onto P81 ion exchange paper.

8. Kinase activity was detected by filter-binding method.

### Results & Discussion

Representative compounds of Formula I were evaluated against WT EGFR and mutant EGFR (L858R and L858R, T790M) kinases. IC₅₀ (nM) concentrations are illustrated in Table 5. This assay confirms that compounds of the application are effective inhibitors of the target EGFR receptors.

TABLE 5

| Example # | EGFR^{WT} | EGFR^{(L858R,T790M)} | EGFR^{(T790M)} |
|---|---|---|---|
| I-2 | 0.0052 | 0.0021 | 16.4 |
| I-3 | 0.0055 | 0.0588 | 5.56 |
| I-5 | 0.0054 | 0.0021 | 0.707 |
| I-7 | 0.0029 | 0.0021 | 10.5 |
| I-8 | 0.0042 | 0.0017 | 79.5 |
| I-9 | 0.0052 | 0.0034 | 0.0597 |
| I-10 | 0.0042 | 0.0027 | 0.1560 |
| I-11 | 0.0452 | 0.0015 | — |
| I-16 | 0.0051 | 0.0032 | 13.7 |
| I-17 | 0.545 | 0.043 | 817 |
| I-18 | 0.0075 | 0.0052 | 62.8 |
| I-22 | 0.5210 | 0.6400 | 13.1 |
| I-25 | 0.0050 | 0.0066 | 0.679 |

### Example 21: Human and Mouse Microsomal Stability

### Protocol

For Phase I analysis, representative compounds of the application (10 mM stock in DMSO) were incubated at a final concentration of 1 μM (this concentration assumed to be well below the Km values to ensure linear reaction conditions). Working stocks were initially diluted to a concentration of 40.0 μM in 0.1 M potassium phosphate buffer before addition to the reaction vials. Pooled mouse (CD-1, male) or human (50 donors) liver microsomes were utilized at a final concentration of 0.5 mg/ml. Duplicate wells were used for each time point (0 and 30 minutes). Reactions were carried out at 37° C. in a shaker, and the final concentration of DMSO was kept constant at 0.01%. The final volume for each reaction was 100 μL, which includes the addition of an NADPH-Regeneration solution (NRS) mix. This NRS mix is comprised of glucose 6-phosphate dehydrogenase (0.4 U/mL), NADP+(1.3 mM), MgCl2 (3.3 mM), and glucose 6-phosphate (3.3 mM) in assay mixtures. Upon completion of the 30 minute time point, reactions were terminated by the addition of 1.5-volumes (150 μL) of ice-cold, acetonitrile with 0.5% formic acid and internal standard. Samples were then centrifuged at 4,000 rpm for 10 minutes to remove debris and precipitated protein. Approximately 150 μL of supernatant was subsequently transferred to a new 96 well microplate for LC/MS analysis.

Narrow-window mass extraction LC/MS analysis was performed for all samples using a Waters Xevo quadrupole time-of-flight (QTof) mass spectrometer and an ACQUITY UPLC system, to determine relative peak areas of parent compound.

$$\% remaining = \frac{Area count of t = 30 min}{Area count of t = 0 min} \times 100$$

### Results & Discussion

Human and mouse liver microsomes contain a wide variety of drug metabolizing enzymes and are commonly used to support in vitro ADME (absorption, distribution, metabolism and excretion) studies. These microsomes are used to examine the potential first-pass metabolism by-products of orally administered drugs. Representative compounds of the application were evaluated for their stability in human and mouse liver microsomes. A majority of the compounds of the application in both human and mouse liver microsomes were recovered within a 60 minute time period indicating that the compounds were not rapidly cleared (see Table 6 for representative compounds of Formula I).

TABLE 6
Metabolic stability of I-9-MA and I-10-MA and control
compounds verapamil in human and mouse with NADPH

| | Remaining Percentage | | | | CLᵢₙₜ (μL/ | |
|---|---|---|---|---|---|---|
| Example | (%) after 60 min | | t_{1/2} (min) | | min/mg protein) | |
| # | Human | Mouse | Human | Mouse | Human | Mouse |
| Verapamil | 4.72 | 1.22 | 13.64 | 9.75 | 101.59 | 142.14 |
| I-9-MA | 47.73 | 30.53 | 59.14 | 35.18 | 23.44 | 39.4 |
| I-10-MA | 60.64 | 48.95 | 86.9 | 57.81 | 15.95 | 23.97 |

### Example 22: Metabolic Stability (Hepatocytes)

### 1—Preparation of Working Solutions

a) Prepare 10 mM stock solutions test compounds and positive control verapamil in DMSO.
b) In separate conical tubes, dilute the 10 mM test compound and the positive control to 100 μM by combining 495 μL of 50% acetonitrile/50% water and 5 μL of 10 mM stock.

### 2—Preparation of Hepatocytes

a) Place incubation medium (William's E Medium supplemented with GlutaMAX) and hepatocyte thawing medium in a 37° C. water bath, and allow warming for at least 15 minutes prior to use.
b) Remove a vial of cryopreserved hepatocytes from storage, ensuring that vials remain at cryogenic temperatures until thawing process ensues. Thaw the cells by placing the vial in a 37° C. water bath and gently shaking the vials for 2 minutes. After thawing is completed, spray vial with 70% ethanol, transfer the vial to a biosafety cabinet.
c) Use wide-bore pipette tip to transfer hepatocytes into 50 mL conical tube containing thawing medium. Place the 50 mL conical tube into a centrifuge and spin at 100 g for 10 minutes. Upon completion of spin, aspirate thawing medium and resuspend hepatocytes in enough incubation medium to yield ˜1.5×10⁶ cells/mL.
d) Using a Trypan Blue exclusion, count cells and determine the viable cell density. Cells with poor viability (<75% viability) are not acceptable for use. Dilute cells with incubation medium to a working cell density of 0.5×10⁶ viable cells/mL.
e) A portion of the hepatocytes at 0.5×10⁶ viable cells/mL was boiled for 5 min prior to adding to the plate as negative control to eliminate the enzymatic activity so that little or no substrate turnover should be observed. The boiled hepatocytes are used to prepare negative control samples, which used to exclude the misleading factor that resulted from instability of chemical itself. Negative control wells are prepared in duplicate.

### 3—Compound Incubation

a) Pipette 198 μL of hepatocytes into each wells of a 96-well non-coated plate. Place the plate in the CO₂ incubator at 37° C. on an orbital shaker to allow the hepatocytes to warm for 10 minutes.
b) Pipette 2 μL of the 100 μM test compounds or positive control into respective wells of the 96-well plate to start the reaction.
c) Return the plate to the incubator and place on an orbital shaker with a shaking speed of 150 rpm. The reaction for each compound will be performed in duplicate.

### 4—Procedure for Stability Determination

a) Remove 25 μL aliquots of reaction mixture at time points of 0, 15, 30, 60, 90 and 120 minutes.
b) The aliquots are then mixed with 6 volumes (150 μL) of acetonitrile containing internal standards (IS: 100 nM alprazolam, 200 nM labetalol, and 2 μM ketoprofen) to terminate the reaction. Centrifuge the plate for 20 minutes at 3,800 g. Aliquots of 100 μL of the supernatants will be mixed with 100 μL ultrapure water and used for LC/MS/MS analysis.

The data in Table 7 shows that the exemplified compounds are not rapidly metabolized and are stable to hepatocyte enzymes.

TABLE 7
Metabolic stability (hepatocytes) results for exemplified compounds

| | Human | Monkey | Dog | Rat | Mouse |
|---|---|---|---|---|---|
| Example # | % Remaining @ 120 min | | | | |
| I-5-MA | 82.95 | 21.97 | 47.29 | 59.08 | 36.27 |
| I-7-MA | 68.69 | 9.47 | 59.76 | 44.39 | 51.8 |
| I-25-MA | 79.87 | 41.03 | 62.17 | 61.57 | 46.51 |

### Example 23: Plasma Protein Binding

### Experimental Procedure

a—Preparation of 100 mM Sodium Phosphate and 150 mM NaCl Buffer (PBS)

Prepare a basic solution by dissolving 14.2 g/L Na₂HPO₄ and 8.77 g/L NaCl in deionized water. Store at 4° C. for up to 7 days. Prepare an acidic solution by dissolving 15.6 g/L NaH₂PO₄.2H₂O and 8.77 g/L NaCl in deionized water. Store at 4° C. for up to 7 days. Titrate the basic solution with the acidic solution to pH 7.4. Store at 4° C. for up to 7 days. Check pH on the day of experiment and adjust if outside specification of 7.4±0.1.

b—Preparation of Dialysis Membranes
Soak the dialysis membranes in ultrapure water for 60 minutes to separate strips, then in 20% ethanol for 20 minutes, finally in dialysis buffer for 20 minutes.
c—Preparation of Plasma

Thaw the frozen plasma immediately in a 37° C. water bath.

Centrifuge plasma at 3,220 g for 10 minutes at room temperature to remove clots and collect supernatant into a fresh tube. Check and record the pH of the plasma.

Note: a) Only use plasma that has been thawed no more than twice since arrival. b) Only use plasma within the range of pH 7 to pH 8.

d—Preparation of Stock Solutions and Working Solutions

The working solutions of test compound and control compound ketoconazole are prepared in 50% water/50% acetonitrile and DMSO at the concentration of 200 μM, respectively. And then remove 3 μL of working solution to mix with 597 μL of human, rat and mouse plasma to achieve final concentration of 1 μM (0.5% DMSO). Vortex thoroughly.

e—Procedure for Equilibrium Dialysis

Assemble the dialysis set up following the manufacturer's instructions. Load cells with 120 μL of plasma sample and dialyzed against equal volume of dialysis buffer (PBS). The assay is performed in duplicate. Seal the dialysis plate and place the plate in an incubator at 37° C. with 5% CO₂ at approximately 100 rpm for 6 hours. At the end of dialysis, remove seal and pipette 50 μL each of post-dialysis samples from both buffer and plasma chambers into separate tubes in plate.

f—Procedure for Sample Analysis

Add 50 μL of blank plasma to the buffer samples, and an equal volume of PBS to the collected plasma samples. Add 300 μL of room temperature quench solution (acetonitrile containing internal standards (IS, 200 nM Labetalol, 100 nM Alprazolam and 2 μM Ketoprofen)) to precipitate protein. Vortex for 5 minutes. Samples in plate are centrifuged at 3,220 g for 30 minutes at room temperature. Transfer 100 μL of the supernatant to a new plate. The supernatant may be diluted with 100 μL or 200 μL water according to the LC/MS signal response and peak shape. Mix well and analyze samples using LC/MS/MS.

Results: Table 8 summarizes Plasma Protein Binding in human, mouse and rat. The data shows that the exemplified compounds are not highly bound to plasma proteins (i.e. <99% bound) across species assessed.

TABLE 8

| | Human | Rat | Mouse |
|---|---|---|---|
| | Plasma | Plasma | Plasma |
| Example # | % Bound | % Bound | % Bound |
| Afatanib | 92 | 91.04 | 93.34 |
| I-7-MA | 92.43 | 94.09 | 94.41 |
| I-5-MA | 88.3 | 92.29 | 90.25 |

### Example 24: CYP450 Inhibition

### Experimental Procedure:

Prepare stock solutions of the test compound and positive control compounds described in Table 8 in DMSO at 0, 0.2, 1, 2, 10, 50, 200, 2000 and 10000 μM. Transfer 1 μL of the stock solutions to the Incubation Plate. The final concentrations of test compound or positive control compound are 0, 0.001, 0.005, 0.01, 0.05, 0.25, 1, 10 and 50 μM. All experiments are performed in duplicate.

TABLE 9
control compounds

| CYP Isoform | Control Compound |
|---|---|
| CYP 1A2 | furafylline |
| CYP 3A4 | ketoconazole |
| CYP 2C9 | sulfaphenazole |
| CYP 2C19 | N-3-benzylnirvanol |
| CYP 2D6 | quinidine |

Results: The results presented in Table 10 shows that representative compounds of the application are not substrates for the tested CYP enzymes.

TABLE 10

| | IC50 (μM) | | | | |
|---|---|---|---|---|---|
| Example # | 1A2 | 2C9 | 2C19 | 2D6 | 3A4-Testosterone |
| Afatinib | >50 | 34.69 | 47.78 | 27.99 | >50 |
| I-7-MA | >50 | 42.90 | 28.56 | 23.19 | 34.93 |
| I-5-MA | >50 | 30.00 | 29.09 | 25.66 | 41.97 |

### Example 25: Chemical Stability

### Experimental Procedure

### Preparation of Test Solutions:

For PBS pH7.4: Prepare 100 mM bulk Na₂HPO₄ solution and 100 mM bulk NaH₂PO₄ solution. Place Na₂HPO₄ solution on a stirrer and slowly add NaH₂PO₄ solution until the pH is nearly 7.4 (±0.05).

For SIF: Dissolve 6.8 g of KH₂PO₄ into about 500 mL ultrapure water and adjust the solution to a pH 6.8 with 0.1 M NaOH. Dissolve 10 g of trypsin into ultrapure water. Then the two solutions are mixed well and diluted with ultrapure water to 1000 mL.

The working solutions of test compound and control compound chlorambucil are prepared in DMSO at the concentration of 500 μM.

Place 2 μL of working solution of each sample in order into their proper 96-well rack. Add 198 μL of pre-incubated PBS pH 7.4 or simulated intestinal fluid into each vial of the cap-less Sample plate to achieve a final concentration of 1 μM. The assay is performed in duplicate. Seal using a molded PTFE/Silicone plug. Then transfer the Sample plate to the Eppendorf Thermomixer Comfort plate shaker and shake at 37° C. at 1100 RPM. Take one of the glass vials at designated time points including 0, 30, 60, 120 and 240 min. For each time point, the initiation of the reaction is staggered so all time points are terminated with 1000 μL of cold quench solution (acetonitrile containing internal standards (IS, 200 nM Labetalol, 100 nM Alprazolam and 2 μM Ketoprofen) at the same time. Vortex for 1 minute. Centrifuge the samples in plate at 3,220 g for 5 minutes at 4° C. Transfer 100 μL of the supernatant to a new plate. The supernatant may be diluted with 100 μL or 200 μL water according to the LC/MS signal response and peak shape. Mix well and analyze samples using LC/MS/MS.

Results: The results shown in Table 11 show that representative compounds of the application are stable at physiological pHs.

TABLE 11

| | |
|---|---|
| | Remaining |
| Remaining | Percentage (%) |
| Percentage (%) | in Simulated |
| in PBS at pH 7.4 | Intestinal Fluid |

| Example# | 120 min | 240 min | 120 min | 240 min |
|---|---|---|---|---|
| Afatinib | 101.12 | 105.37 | 104.51 | 104.31 |
| I-7-MA | 100.99 | 100.66 | 105.51 | 110.82 |
| I-5-MA | 94.70 | 91.17 | 97.51 | 91.51 |

### Example 26: Plasma Stability

### Experimental Procedure

### 1) Preparation of Plasma

Thaw the frozen plasma immediately in a 37° C. water bath. Centrifuge plasma at 3,220 g for 10 minutes at room temperature to remove clots and collect supernatant into a fresh tube. Check and record the pH of the plasma. (Note: a. Only use plasma that has been thawed no more than twice since arrival. b. Only use plasma within the range of pH 7 to pH 8.)

### 2) Preparation of Working Solutions

The working solutions of test compounds and control compound are prepared in 50% water/50% acetonitrile and DMSO at the concentration of 100 μM, respectively. Propantheline is used as positive control for human and mouse plasma stability assay and mevinolin is used as the positive control for dog and rat plasma stability assay.

### 3) Procedure for Stability Determination

Add 5 μL of 100 μM working solution to 495 μL of pre-incubated human, monkey, dog, rat or mouse plasma to reach a final concentration of 1 μM. The final concentration of organic solvents is 0.5%. The assay is performed in duplicate. Incubate the reaction samples at 37° C. water bath with shaking at approximately 60 rpm. Take aliquots of 50 μL from the reaction samples at 0, 15, 30, 45, 60 and 120 minutes. Stop the reaction by adding 300 μL of room temperature quench solution (Acetonitrile containing internal standards (IS, 200 nM Labetalol, 100 nM Alprazolam and 2 μM Ketoprofen)). Vortex for 5 minutes. Centrifuge samples in plate at 3,220 g for 30 minutes at room temperature to precipitate protein. Transfer 100 μL of the supernatant to a new plate. The supernatant may be diluted with 100 μL or 200 μL water according to the LC/MS signal response and peak shape. Mix well and analyze samples using LC/MS/MS.

Results: The results shown in Table 12 show that representative compounds of the application are stable in mammalian plasma.

TABLE 12

| | Human | | Monkey | | Dog | |
|---|---|---|---|---|---|---|
| | Remaining | | Remaining | | Remaining | |
| Example | Percentage (%) | | Percentage (%) | | Percentage (%) | |
| # | 60 min | 120 min | 60 min | 120 min | 60 min | 120 min |
| Afatinib | 94.78 | 98.62 | 102.98 | 97.24 | 111.61 | 102.15 |
| I-5-MA | 101.95 | 95.45 | 85.79 | 83.32 | 104.57 | 102.13 |
| I-7-MA | 114.10 | 96.37 | 88.33 | 102.35 | 105.43 | 91.93 |

| | |
|---|---|
| Rat | Mouse |
| Remaining | Remaining |
| Percentage (%) | Percentage (%) |

| Compounds | 60 min | 120 min | 60 min | 120 min |
|---|---|---|---|---|
| Afatinib | 101.43 | 95.31 | 101.81 | 106.59 |
| I-5-MA | 99.28 | 95.14 | 95.45 | 105.25 |
| I-7-MA | 102.47 | 100.34 | 104.36 | 109.90 |

### Example 27: Log D Determination

### Experimental Procedure

### 1—Preparation of Stock Solutions

Prepare the stock solutions of test compounds and control compound progesterone in DMSO at the concentration of 30 mM. If test compound could not be dissolved completely, make a 10 mM stock solution with DMSO.

### 2—Procedure for Log D Determination

Place 5 μL of stock solution (30 mM) or 15 μL of stock solution (10 mM) of each sample in order into their proper 96-well rack. Add 500 μL of PBS pH 7.4 into each vial of the cap-less Log D plate followed by the addition of 500 μL of 1-octanol. Add one stir stick to each vial and seal using a molded PTFE/Silicone plug. Then transfer the Log D plate to the Eppendorf Thermomixer Comfort plate shaker and shake at 25° C. at 1,100 RPM for 1 hour. After completion of incubation, remove the stir sticks using a big magnet. Then centrifuge the samples at 25° C. at 20,000 g for 20 minutes to separate the phases, and use pipette and syringe to remove the upper (1-octanol) and lower (buffer) phases to the empty tubes, respectively. Take aliquots of 5 μL from upper phases followed by addition of 495 μL of methanol. Vortex for 1 minute, and then take aliquots of 50 μL from the diluent followed by addition of 450 μL of methanol. And take aliquots of 50 μL from lower phases followed by addition of 450 μL of methanol. A certain proportion of ultrapure water may be used according to the peak shape. The dilution factor may be changed according to the Log D value and the LC/MS signal response.

Results: Representative compounds of the application showed a desirable Log D value (<4.5 for ideal drugs).

TABLE 13

LogD Value

| Example# | pH 2.0 | pH 7.4 |
|---|---|---|
| Afatinib | −2.46 | 3.18 |
| I-7-MA | −1.57 | 3.42 |
| I-5-MA | −2.17 | 3.20 |

### Example 28: Log P Determination

### Experimental Procedure

### 1—Preparation of Unretained Organic Substance Sample and Reference Substances Mixture

Prepare thiourea in acetonitrile and ultrapure water mixture (1:1 in v:v) as unretained organic substance sample. Prepare acetanilide, methyl benzoate, benzophenone, benzyl benzoate, fluoranthene and DDT in acetonitrile and ultrapure water mixture (1:1 in v:v) as reference substances mixture.

### 2—Preparation of Test Samples

Prepare the stock solutions of test compounds and control compound progesterone in acetonitrile at the concentration of 2 mM. Dilute one volume of the stock solution with one volume of ultrapure water. If not soluble, filtrate the sample using a 0.45 μm nylon membrane.

### 3—Procedure for Log P Determination

Prepare acidic or basic mobile phase according to the character of test compounds. Run a blank sample (ultrapure water mixture (1:1 in v:v)), followed by unretained organic substance sample and reference substances mixture. Then run the test compounds and control compound in singlet, and at the end of the batch rerun the reference substances mixture.

Results: Representative compounds of the application showed a desirable Log P value (<4.5 for ideal drugs).

TABLE 14

| Example# | Log P Value |
|---|---|
| Afatinib | 4.26 |
| I-7-MA | 3.56 |
| I-5-MA | 3.57 |

### Example 29: Plasma and Brain Exposure of 1-9, 1-10 and Afatinib in Male CD-1 Mice

Protocol

Animals: Male CD-1 mice (20-25 g) from Charles River Labs will be acclimatized for a minimum of 5 days prior to dosing. Body weights will be recorded on the day of dosing.
Food restriction: Animals dosed p.o. will be deprived of food overnight and fed ˜2 h following dosing.
Clinical observations: Animals will be observed at the time of dosing and each sample collection. Any abnormalities will be documented.
Dosing: Formulations will be administered i.v. via the tail vein and p.o. by gavage with disposable feeding needles.
Sample collection: Terminal blood samples will be collected under O2/CO2 anesthesia by cardiac puncture and then the brain and lungs will be excised blotted and weighed.
Sample processing/storage: All blood samples will be transferred into K2EDTA tubes and centrifuged (6,800 rpm for 5 min at 4° C.) to obtain plasma. All samples will be stored frozen at −80° C. until bioanalysis.
Sample retention: Plasma, brain and lung samples will be analyzed and any remaining samples will be stored frozen at −80° C. until the study is completed.

TABLE 15
Experimental parameters

| Group | Example | | #/sex of | Dose | Conc | Volume | Sample |
|---|---|---|---|---|---|---|---|
| ID | # | Route | animals | (mg/kg free base) | (mg/mL free base) | (mL/kg) | Collection |
| 1 | Afatinib | i.v. | 24 M | 1 | 0.2 | 5 | Terminal |
| 2 | | p.o. | 24 M | 25 | 2.5 | 10 | blood, |
| 3 | I-9-MA | i.v. | 24 M | 1 | 0.2 | 5 | brain and |
| 4 | | p.o. | 24 M | 25 | 2.5 | 10 | lung |
| 5 | I-10-MA | i.v. | 24 M | 1 | 0.2 | 5 | (3 mice/time- |
| 6 | | p.o. | 24 M | 25 | 2.5 | 10 | point) |

TABLE 16
Sample collection

| | Sample collection time | Volume/animal/time- |
|---|---|---|
| Group ID | (h) | point |
| 1, 3, 5 | 0.0833, 0.25, 0.5, 1, 2, 4, | ~0.6 mL blood; brain and |
| | 8 & 24 | lungs will be weighed |
| 2, 4, 6 | 0.25, 0.5, 1, 2, 4, 6, 8 & 24 | |

Results: Table 17 summarizes the pharmacokinetic profile in brain and plasma. As can be seen, representative compounds of the application showed significantly higher concentrations in the brain after both intravenous and oral administration as evidenced by the Cmax and AUC values of the compounds of Formula I compared with Afatinib. Further the ratio of brain/plasma AUC was significantly higher for the compounds of the application compared to Afatinib.

TABLE 17
Brain PK

| | | I-9-MA | | I-10-MA | | Afatinib | |
|---|---|---|---|---|---|---|---|
| Parameter | Unit | i.v. | p.o. | i.v. | p.o. | i.v. | p.o. |
| tₘₐₓ | h | 0.0833 | 2.00 | 0.250 | 2.00 | 0.0833 | 4.00 |
| Cₘₐₓ | ng/mL | 171.0 | 651 | 1110 | 3833 | 12.7 | 83.3 |
| Apparent t_{1/2} | h | 7.11^{a} | 3.44 | 4.75^{a} | 3.25^{a} | 4.97 | 3.69^{a} |
| AUC₀₋ₜₗₐₛₜ | h*ng/mL | 397 | 5905 | 1713 | 13944 | 44.5 | 688 |
| AUC_{0-inf} | h*ng/mL | 420 | 5963 | 1729 | 13981 | 65.6 | 704 |
| MRT₀₋ₜₗₐₛₜ | h | 6.60 | 6.78 | 2.99 | 3.68 | 7.01 | 8.64 |
| Brain/Plasma | n/a | 1.09 | 0.526 | 1.16 | 0.845 | 0.758 | 0.130 |
| AUC_{0-inf} | | | | | | | |

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A compound of Formula I or a pharmaceutically acceptable salt, solvate and/or prodrug thereof: 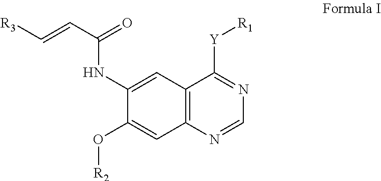
wherein:
R₁ is selected from aryl and heteroaryl both of which are unsubstituted or substituted with one or more substituents selected from halo, C₁₋₆alkyl, haloC₁₋₆alkyl, C₂₋₆alkenyl, C₂₋₆alkynyl, CN, CF₃, OR₆, SR₆, N(R₆)₂, and 3-7 membered heterocycloalkyl;
each R₆ is independently H, aryl, heteroaryl, C₁₋₆alkyl, haloC₁₋₆alkyl, C₂₋₆alkenyl or C₂₋₆alkynyl;
R₂ is selected from C₁₋₆alkyl, 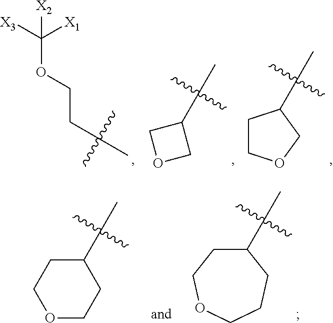
X₁, X₂ and X₃ are the same or different and are selected from H, halo and C₁₋₆alkyl
R₃ is selected from H, 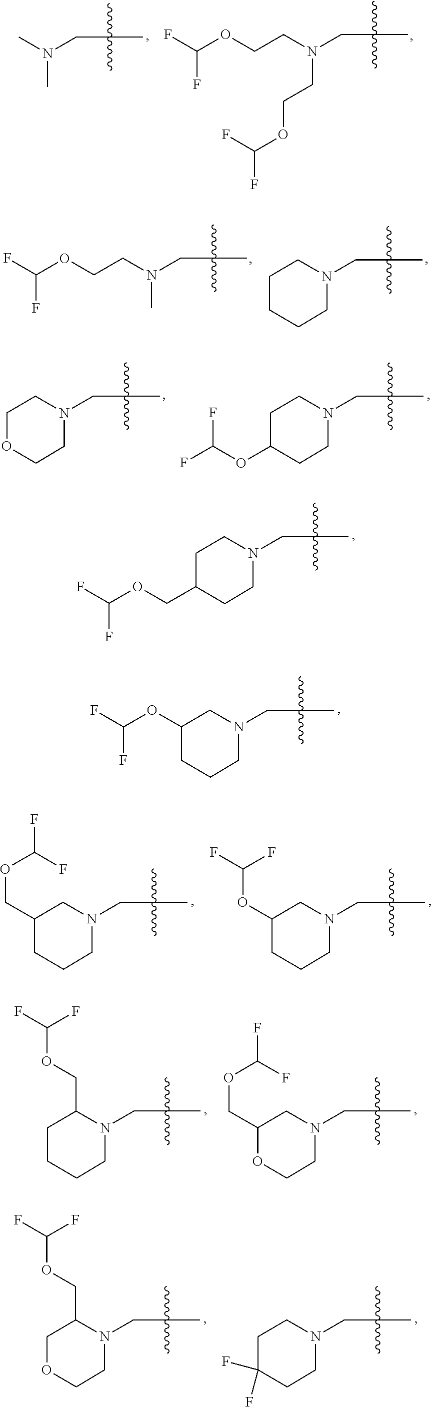 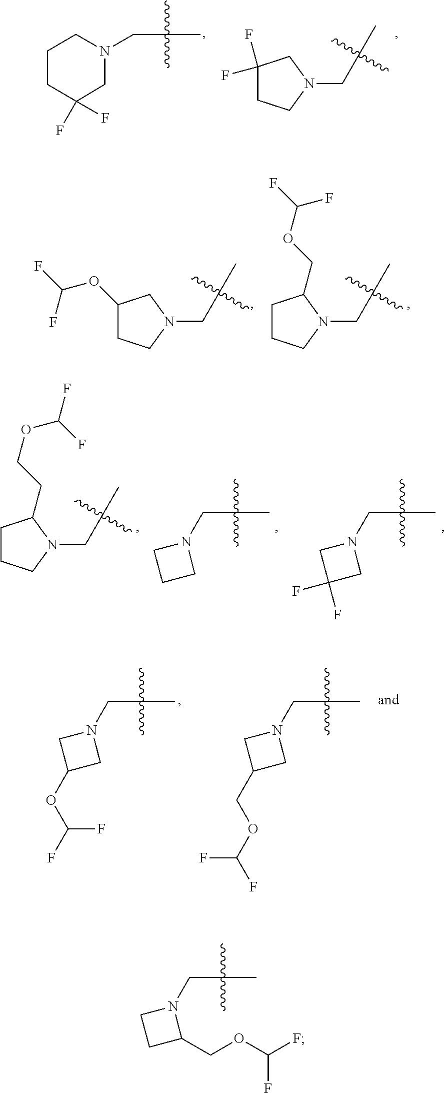
Y is selected from NH, O, S, SO and SO₂; and
at least one of R₂ and R₃ comprises a difluoromethyl group or at least R₃ comprises difluoromethylene group.

2. (canceled)

3. The compound of claim 1, wherein R₁ is selected from unsubstituted or substituted aryl wherein the substituents for R₁ are selected from one to four of one to four of halo, C₁₋₆alkyl, haloC₁₋₆alkyl, CN, OR₆, N(R₆)₂, C₂₋₆alkynyl, and 5-6 membered heterocycloalkyl, in which R₆ is selected from haloC₁₋₆alkyl and C₁₋₆alkyl.

4. (canceled)

5. The compound of claim 1, wherein R₁ is substituted phenyl wherein the substituents of R₁ are selected from one to three of Cl, F, CF₃, OR₆, N(R₆)₂, C₂₋₆alkynyl, in which R₆ is selected from CF₃, CHF₂ and CH₃.

6. (canceled)

7. The compound of claim 1, wherein R₁ is selected from

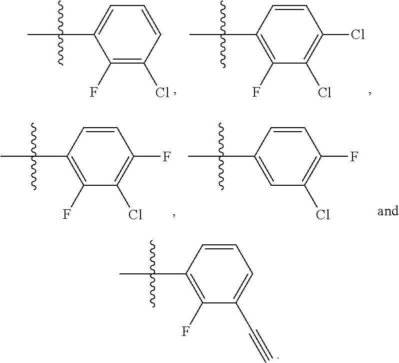

8. (canceled)

9. The compound of claim 1, wherein R₁ is substituted heteroaryl wherein the substituents of R₁ are selected from one to three of Cl, F, CF₃, OR₆, N(R₆)₂, C₂₋₆alkynyl, in which R₆ is selected from fluoroC₁₋₆alkyl and C₁₋₆alkyl.

10. The compound of claim 1, wherein R₂ is selected from C₁₋₆alkyl and

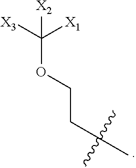

11. The compound of claim 10, wherein R₂ is

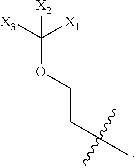

12. (canceled)

13. The compound of claim 11, wherein X₁, X₂ and X₃ are the same or different and are selected from H, F and C₁₋₄alkyl.

14. (canceled)

15. The compound of claim 13, wherein at least one of X₁, X₂ and X₃ is F.

16. The compound of claim 1, wherein R₃ is selected from:

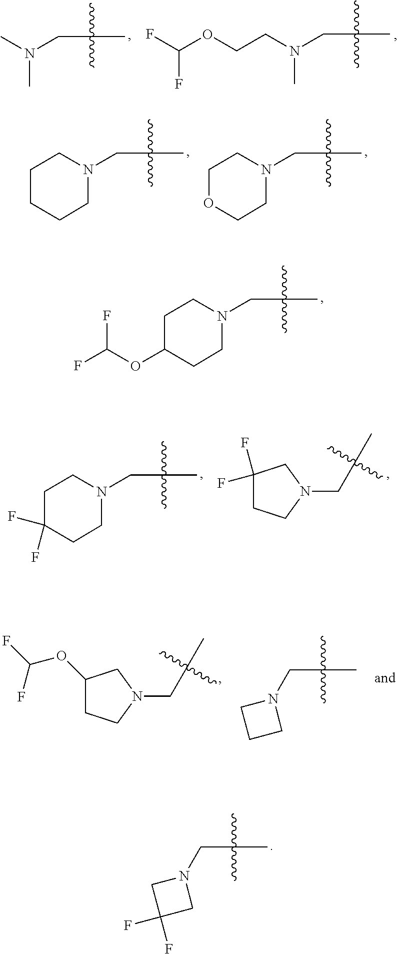

17. The compound of claim 16, wherein R₃ is selected from:

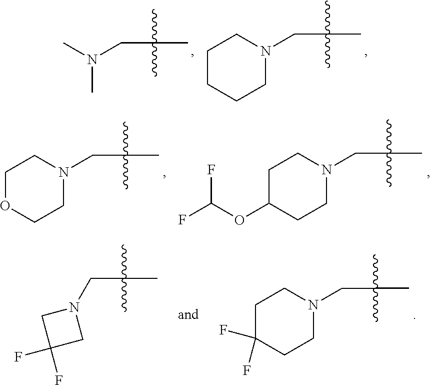

18. (canceled)

19. (canceled)

20. (canceled)

21. The compound of claim 1, wherein at least one of R₂ and R₃ comprises

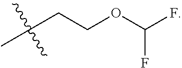 [SMILES: *CCOC(F)F]

22. The compound of claim 1, wherein Y selected from O, NH and NCH₃.

23. (canceled)

24. (canceled)

25. The compound of claim 1, selected from:
N-[4-(3,4-dichloro-2-fluoro-anilino)-7-[2-(difluoromethoxy)ethoxy]quinazolin-6-yl]prop-2-enamide;
(E)-N-[4-(3,4-dichloro-2-fluoro-anilino)-7-[2-(difluoromethoxy) ethoxy]-quinazolin-6-yl]-4-(dimethylamino)but-2-enamide;
(E)-N-[4-(3,4-dichloro-2-fluoro-anilino)-7-[2-(difluoromethoxy)ethoxy]-quinazolin-6-yl]-4-morpholino-but-2-enamide;
(E)-N-[4-(3-chloro-2-fluoro-anilino)-7-[2-(difluoromethoxy)ethoxy]quinazolin-6-yl]-4-(dimethylamino)but-2-enamide;
(E)-N-[4-(3-chloro-2-fluoro-anilino)-7-[2-(difluoromethoxy)ethoxy]quinazolin-6-yl]-4-(1-piperidyl)but-2-enamide;
(E)-N-[4-(3-chloro-2-fluoro-anilino)-7-[2-(difluoromethoxy)ethoxy]quinazolin-6-yl]-4-morpholino-but-2-enamide;
(E)-N-[4-(3-chloro-2-fluoro-anilino)-7-[2-(difluoromethoxy)ethoxy]quinazolin-6-yl]-4-[4-(difluoromethoxy)-1-piperidyl]but-2-enamide;
(E)-N-[4-(3-chloro-2,4-difluoro-anilino)-7-[2-(difluoromethoxy) ethoxy]-quinazolin-6-yl]-4-(dimethylamino)but-2-enamide;
(E)-N-[4-(3-chloro-2,4-difluoro-anilino)-7-[2-(difluoromethoxy) ethoxy]-quinazolin-6-yl]-4-(1-piperidyl)but-2-enamide;
(E)-N-[4-(3-chloro-2,4-difluoro-anilino)-7-[2 (difluoromethoxy) ethoxy]-quinazolin-6-yl]-4-morpholino-but-2-enamide;
(E)-N-[4-(3-chloro-2,4-difluoro-anilino)-7-[2 (difluoromethoxy) ethoxy]quinazolin-6-yl]-4-[4-(difluoromethoxy)-1-piperidyl]but-2-enamide;
(E)-N-[4-(3,4-dichloro-2-fluoro-anilino)-7-[2-(difluoromethoxy)-ethoxy]quinazolin-6-yl]-4-(1-piperidyl)but-2-enamide;
(E)-N-[4-(3,4-dichloro-2-fluoro-anilino)-7-[2-(difluoromethoxy) ethoxy]-quinazolin-6-yl]-4-[4-(difluoromethoxy)-1-piperidyl]-but-2-enamide;
(E)-N-[4-(3-chloro-2-fluoro-anilino)-7-[2-(difluoromethoxy)ethoxy]quinazolin-6-yl]-4-(3,3-difluoroazetidin-1-yl)but-2-enamide;
(E)-N-[4-(3-chloro-2,4-difluoro-anilino)-7-[2-(difluoromethoxy)-ethoxy]quinazolin-6-yl]-4-(3,3-difluoroazetidin-1-yl)but-2-enamide;
(E)-N-[4-(3,4-dichloro-2-fluoro-anilino)-7-methoxy-quinazolin-6-yl]-4-[4-(difluoromethoxy)-1-piperidyl]but-2-enamide;
(E)-N-[4-(3-chloro-4-fluoro-anilino)-7-methoxy-quinazolin-6-yl]-4-[4-(difluoromethoxy)-1-piperidyl]but-2-enamide;
(E)-N-[4-(3-chloro-4-fluoro-anilino)-7-[2-(difluoromethoxy)ethoxy]quinazolin-6-yl]-4-(dimethylamino)but-2-enamide;
(E)-N-[4-(3-chloro-4-fluoro-anilino)-7-[2-(difluoromethoxy)ethoxy]quinazolin-6-yl]-4-(1-piperidyl)but-2-enamide;
(E)-N-[4-(3-chloro-4-fluoro-anilino)-7-[2-(difluoromethoxy)ethoxy]quinazolin-6-yl]-4-morpholino-but-2-enamide;
(E)-N-[4-(3-chloro-4-fluoro-anilino)-7-[2-(difluoromethoxy)ethoxy]quinazolin-6-yl]-4-[4-(difluoromethoxy)-1-piperidyl]but-2-enamide;
(E)-N-[4-(3-chloro-4-fluoro-anilino)-7-[2-(difluoromethoxy)ethoxy]quinazolin-6-yl]-4-(3,3-difluoroazetidin-1-yl)but-2-enamide;
(E)-N-[4-(3-chloro-4-fluoro-anilino)-7-[2-(difluoromethoxy)ethoxy]quinazolin-6-yl]-4-(4,4-difluoro-1-piperidyl)but-2-enamide;
(E)-N-[7-[2-(difluoromethoxy)-ethoxy]-4-(3-ethynyl-2-fluoro-anilino)quinazolin-6-yl]-4-(dimethylamino)but-2-enamide;
(E)-N-(4-(3-chloro-2-fluorophenoxy)-7-(2-(difluoromethoxy)ethoxy)quinazolin-6-yl)-4-(dimethylamino)but-2-enamide;
(E)-N-(4-(3-chloro-4-fluorophenoxy)-7-(2-(difluoromethoxy)ethoxy)quinazolin-6-yl)-4-(dimethylamino)but-2-enamide;
(E)-N-(4-(3-chloro-2-fluorophenoxy)-7-(2-(difluoromethoxy)ethoxy)quinazolin-6-yl)-4-(piperidin-1-yl)but-2-enamide;
(E)-N-(4-(3-chloro-2-fluorophenoxy)-7-(2-(difluoromethoxy)ethoxy)quinazolin-6-yl)-4-morpholinobut-2-enamide; and
(E)-N-(4-(3-chloro-2-fluorophenoxy)-7-methoxyquinazolin-6-yl)-4-(dimethylamino)but-2-enamide;
or a pharmaceutically acceptable salt and/or hydrate thereof.

26. A compound of claim 1 that is: 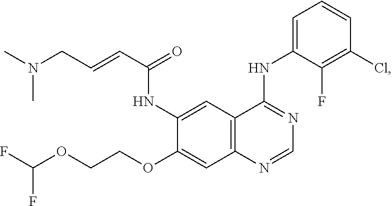 [SMILES: CN(C)C/C=C/C(=O)Nc1cc2c(Nc3cccc(Cl)c3F)ncnc2cc1OCCOC(F)F]
or a pharmaceutically acceptable salt and/or solvate thereof, or 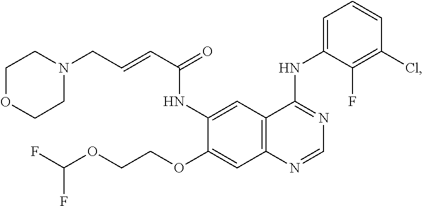 [SMILES: O=C(/C=C/CN1CCOCC1)Nc1cc2c(Nc3cccc(Cl)c3F)ncnc2cc1OCCOC(F)F]
or a pharmaceutically acceptable salt and/or solvate thereof.

27. (canceled)

28. A compound of claim 1 that is: 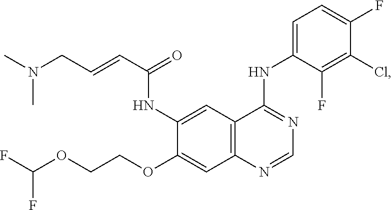 [SMILES: CN(C)C/C=C/C(=O)Nc1cc2c(Nc3ccc(F)c(Cl)c3F)ncnc2cc1OCCOC(F)F]
or a pharmaceutically acceptable salt and/or solvate thereof, or 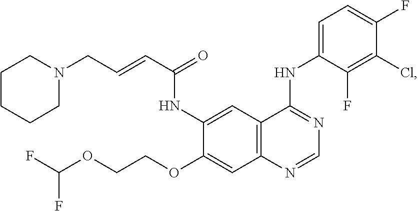 [SMILES: O=C(/C=C/CN1CCCCC1)Nc1cc2c(Nc3ccc(F)c(Cl)c3F)ncnc2cc1OCCOC(F)F]
or a pharmaceutically acceptable salt and/or solvate thereof.

29. (canceled)

30. A pharmaceutical composition comprising one or more compounds of claim 1, or a pharmaceutically acceptable salt, and/or solvate thereof, and a pharmaceutically acceptable carrier and/or diluent.

31. (canceled)

32. A method of treating one or more diseases, disorders or conditions treatable by inhibition of EGFR comprising administering an effective amount of one or more compounds of claim 1, or a pharmaceutically acceptable salt, and/or solvate thereof, to a subject in need thereof.

33. The method of claim 32, wherein the disease, disorder or condition is a cancer.

34. (canceled)

35. The method of claim 33, wherein the cancer is selected from breast cancer, skin cancer, prostate cancer, colon cancer, pancreatic cancer, kidney cancer, ovarian cancer, lung cancer and brain cancer.

36. (canceled)
